(19) United States
(12) Patent Application Publication
(10) Pub. No.: US 2014/0066523 A1
(43) Pub. Date: Mar. 6, 2014

(54) Drug Delivery Compositions And Methods Of Use

(71) Applicants: David Knaack, Summit, NJ (US); Ankur Gandhi, Metuchen, NJ (US); Jordan Katz, Short Hills, NJ (US); Marci Wirtz, Bronx, NY (US); Richard L. Kronenthal, Fair Lawn, NJ (US)

(72) Inventors: David Knaack, Summit, NJ (US); Ankur Gandhi, Metuchen, NJ (US); Jordan Katz, Short Hills, NJ (US); Marci Wirtz, Bronx, NY (US); Richard L. Kronenthal, Fair Lawn, NJ (US)

(73) Assignee: ABYRX, Inc., Irvington, NY (US)

(21) Appl. No.: 14/115,219

(22) PCT Filed: May 1, 2012
(86) PCT No.: PCT/US2012/035975

§ 371 Date: Nov. 1, 2013

Related U.S. Application Data
(60) Provisional application No. 61/481,889, filed on May 3, 2011.

(51) Int. Cl. A61K 47/34

(52) U.S. Cl. CPC A61K 47/34
USPC 514/772.7

(57) ABSTRACT

The invention provides moldable drug delivery carriers made up of a suspension of a solid phase and an organic liquid phase for the sustained release of a therapeutic agent. The invention also provides multiphase drug delivery systems made up of a granular hydrophobic solid phase, an organic liquid phase and a hydrogel, for sustained drug delivery at varying rates over the life of the composition.

### BACKGROUND OF THE INVENTION

In many clinical circumstances, site specific delivery of a therapeutic agent within the body is advantageous compared to systemic delivery strategies. The application of a therapeutic moiety directly to the target site can eliminate unwanted systemic side effects due to the action of the therapeutic moiety at a site other than its intended target. Approaches to the site specific delivery of therapeutic agents through implantable or injectable vehicles have traditionally taken one of five forms: 1.) Injectable micellar or liposomal particulates, 2.) delivery by means of mechanical or osmotic transdermal or in dwelling pumps, 3.) erodible delivery vehicles, 4.) sol-gel systems, or 5.) monolithic hydrogels. To date, group 1 has found greater application in the systemic delivery of drugs as opposed to local delivery. Of the remaining groups, only group 2 has seen commercialization to an appreciable extent.

While referred to as “particulate” systems, micellar and liposomal delivery strategies employ amphipathic oils to produce multiphasic aqueous suspensions of lipid droplets. The complex nature of micellar and liposomal systems which combine hydrophilic and hydrophobic phases, makes them difficult to manufacture and leads to complex delivery kinetics resulting in significant sterilization and storage stability issues. Following injection, it is also difficult to retain these systems at the local site where drug delivery is desired.

Approaches involving pumps have the obvious draw backs of being either transdermal and thereby serving as a conduit for the introduction of infective microbes or, in the case of implantable pumps, of generally not being absorbable. Of the remaining approaches each has its own specific drawbacks. Patches, erodible vehicles, sol-gel systems, hydrogel delivery systems, hydrophobic liquid injectable compositions, and non particulate solid implantable compositions all share limitations such as instability, and/or complexity of manufacture. The majority of local drug delivery systems described in the art incorporate water which may often lead to significant issues in shelf life stability and/or sterilization with ionizing radiation. Kronenthal et al., (U.S. Pat. No. 4,568,536) disclose a drug delivery system which delivered an anti-infective compound for longer than 15 days with very low elution rates of less than 40 microgram per day.

The invention described herein provides non-erosion-based substantially anhydrous delivery systems comprising a suspension of largely insoluble micronized particles within a mobile vehicle comprising substituents of differing polarities and or solvation properties to yield absorbable, highly tunable drug delivery rates and in vivo absorption that represents substantially different technology from the so called liposomal and micellar particulate systems, and without their complications and drawbacks. The inventive systems are capable of providing higher hourly release rates of drugs than those disclosed by Kronenthal.

### SUMMARY OF THE INVENTION

The present invention provides a drug delivery carrier comprising a suspension of a micronized solid and a mobile phase that is a solid or liquid having low water solubility, wherein the micronized solid is substantially insoluble in the mobile phase and makes up between 10 and 70% of the composition, and wherein the mobile phase comprises a surfactant having a hydrophile-lipophile balance (HLB) value that is less than 17, wherein the elution rate of a drug substance from the carrier in an in vitro test is greater than 1.0 mg/hr for at least 45 hours under conditions in which the carrier is mixed with 16% (wt/wt) of the drug substance and eluted for 72 hours in a phosphate buffer at 37 C. In one embodiment, the drug substance in the in vitro test is lidocaine free base.

In one embodiment, the surfactant is a neutral surfactant. In one embodiment, the neutral surfactant is one or more surfactants selected from the group consisting of sorbitan, polyacrylates, alkoxylated fatty alcohols, block copolymers of polypropylene oxide and ethylene oxide, ethylene glycol, propylene glycol, blocked polymers (e.g., Chemal BP 261), silicon glycol co-polymers, polyoxyethylene ethers, ethoxylated triglycerides, ethoxylated fatty acids, ethoxylated fatty amines, and derivatives and modifications of any of the foregoing. In one embodiment, the neutral surfactant is an alkylene oxide block copolymer selected from the group consisting of Pluronic L-10, Pluronic L-43, Pluronic L-44, Pluronic L-61, Pluronic L-62, Pluronic 17R2, and Pluronic L-92.

In one embodiment, the neutral surfactant is a liquid with a critical micelle concentration (CMC) of less than 1%. In one embodiment, the surfactant is selected from the group consisting of poloxamers, sorbitan derivatives and ethoxylated fatty acids.

In one embodiment, the mobile phase comprises a poloxamer. In one embodiment, the mobile phase comprises a reservoir having low water solubility. In one embodiment, the reservoir is selected from the group consisting of sucrose acetate isobutyrate (SAIB), alpha-tocopherol acetate, α-tocopherol, pegylated tocopherol succinate, sorbitan oleate, sorbitan laurate, vitamin K1, cholesterol, fatty acid esters, block copolymers of polypropylene oxide and ethylene oxide (e.g., Pluronic L-31, Pluronic L-81, Pluronic L-101, Pluronic 31R1, Pluronic L-121), alkoxylated fatty alcohols, sorbitan trioleate, and polypropylene glycol 2000.

In one embodiment, the micronized solid is selected from the group consisting of an anionic material, a cationic material, or a porous material. In one embodiment, the micronized solid is an anionic material and wherein the anionic moiety is selected from the group consisting of carboxylates, phosphates, sulfates, carbonates, phosphonates, silicates, and chlorates. In one embodiment, the micronized solid is an cationic material and wherein the cationic moiety is selected from the group consisting of amines, ammonium, and choline. In one embodiment, the micronized solid is a porous material selected from the group consisting of ceramics, polysaccharides, fatty acid salts and polyamines. In one embodiment, the porous material is a calcium, magnesium or zinc salt of a fatty acid selected from the group consisting of stearate, palmitate, or laurate.

Preferably, the micronized solid has at least one property selected from the group consisting of hydrophobicity, osteoconductivity, osteoinductivity, water absorptivity, procoagulation, porosity, and ionic charge. In certain embodiments, an osteoconductive micronized solid is selected from the group consisting of ceramics, synthetic polymers, calcium phosphates, (octacalcium phosphates, hydroxyapatite (HA), HA/TCP), substituted calcium phosphates (silicate-, strontium- and magnesium-substituted), calcium carbonates, calcium sulfates, magnesium phosphates, aluminum phosphates, glasses, phosphate glasses, bioglasses, and tissue-derived particles. In certain embodiments, a substantially hydrophobic micronized solid is selected from the group consisting of synthetic polymers, steroidal compounds, cholesterol and cholesterol derivatives, carboxylic acid salts, phospholipid salts, and salts of phosphatidic acid, and derivatives and modifications thereof. In certain embodiments, the micronized solid is selected from the group consisting of insoluble acyl glycerols and glycerol phosphates, poly lactides, poly galactides, tyrosine polycarbonates, tyrosine polyarylates, absorbable polyurethanes, fumerates, insoluble Pluronics (poloxamers), pegylated protein-based polymers, polyethylene glycols, and particulate ceramics.

In one embodiment, the drug delivery carrier further comprises an embedded solid ceramic material.

In one embodiment, the micronized solid comprises a drug substance in an amount up to 25% by weight of the total weight of the micronized solid. In one embodiment, the drug substance is selected from the group consisting of the group consisting of bone growth enhancers, anti-inflammatory agents, anticancer therapeutics, anesthetics, analgesics, antimicrobials, antiseptics, nucleic acids, transcription activators, peptide growth factors, neuropeptides, neuromodulators, hormones, vitamins, and antiarythmics. In one embodiment, the drug substance is not an analgesic or an anesthetic.

In one embodiment, the drug substance is an agent depot selected from the group consisting of bone growth enhancers, anti-inflammatory agents, anticancer therapeutics, anesthetics, antimicrobials, antiseptics, nucleic acids, transcription activators, peptide growth factors, transcription activators, SniRNA, neuropeptides, neuromodulators, hormones, vitamins, and antiarhythmics. In one embodiment, the drug substance is a bone growth enhancer selected from the group consisting of statins, atorvastatin, cerivastatin, fluvastatin, lovastatin, mevastatin, pitavastatin, pravastatin, rosuvastatin, simvastatin, simvastatin, lovastatin, niacin, amlodipine besylate, Vitamin D, calcitonin, serotonin, serotonin uptake inhibitors, insulin, insulin like growth factors, BMP, calcitriol, calcidiol, growth hormone, PTH (teraparatide), sodium fluoride, PDGF, prostaglandin E1, bisphosphonates, substance P, and CGRH. In one embodiment, the drug substance is an anesthetic selected from the group consisting of procaine, tetracaine, amethocaine, cocaine, lidocaine, prilocaine, bupivacaine, levobupivacaine, ropivacaine, dibucaine, thiopental, methohexital, midazolam, lorazepam, diazepam, propofol, etomidate, ketamine, fentanyl, alfentanil, sufentanil, remifentanil, buprenorphine, butorphanol, diamorphine, hydromorphone, levorphanol, meperidine, methadone, morphine, nalbuphine, oxycodone, oxymorphone, flecanide, benzocaine, phenyloin, pentacaine, heptacaine, carbisocane, isoflurane, methoxyflurane, tocamidew, quinidine, mexiletine, alfaxalone, butamben, enflurane, sevoflurane and pentazocine, phenyloin, pentacaine, heptacaine, carbisocaine, isoflurane, methoxyflurane, tocanide, quinidine, mexilitine, alfaxalone, propofol, butamben, enflurane, and sevoflurane. In one embodiment, the drug substance is an antiinfective agent selected from the group consisting of beta lactams, cephalosporins, silver compounds, peptide antimicrobials, triclosan, gentamicin, tobramycin, silver, silver stearate and silver salts of fatty acids and lipids, ceftazidine, fluconozole, tetracycline, vancomycin, cephalexin, methicillin, gramicidin, minocycline and rifampin.

In one embodiment, the drug delivery carrier further comprises an osteoconductive component.

In one embodiment, the drug delivery carrier further comprises a substantially anhydrous hydrogel forming material.

In one embodiment, the drug delivery carrier further comprises a second drug-containing solid material. In one embodiment, the second drug-containing solid material is selected from the group consisting of a porous ceramic, an erodible polymer, a solid surfactant, a substantially anhydrous hydrogel forming material, and a waxy solid.

In one embodiment, less than 84% of the drug substance is eluted within the 72 hours of the in vitro test. In one embodiment, the burst elution rate of the drug substance during the first hour of release is less than 20 mg/hr in the in vitro test. In one embodiment, less than 84% of the drug substance is eluted within the 72 hours of the in vitro test and the release rate in the first three days is greater than 40 micrograms per day.

The invention also provides a method for delivering a drug substance to a tissue, the method comprising contacting a drug delivery carrier of the invention containing the drug substance with the tissue, thereby delivering the drug substance to the tissue. In one embodiment, the tissue is a soft tissue or a hard tissue.

The invention also provides a method for reducing or stopping the flow of blood from a tissue, the method comprising contacting the tissue with a drug delivery carrier of the invention, thereby reducing or stopping the flow of blood from the tissue. In one embodiment, the tissue is a soft tissue or a hard tissue. In one embodiment, the tissue is a hard tissue and the hard tissue is bone.

The invention also provides a method for producing a local nerve block, the method comprising the step of placing the drug delivery carrier of the invention adjacent to a nerve of interest thereby producing a local nerve block.

### BRIEF DESCRIPTION OF THE DRAWINGS

FIG. 1: Device Components. Shown is a schematic depicting the possible Agent Depot (AD) release routes from the device (control points 1 & 2) and interactions of device components exploitable to alter the AD delivery kinetics. Control point 1 represents AD released from the device by direct solubilization from the device into the aqueous milieu. Control point 2 represents AD released from the device in conjunction with the solublization of all or part of the mobile phase. The rate of release of the AD from the device is controlled through the direct manipulation of either or both of the release routes (control points 1 and 2), through manipulation of one or more interactions between the device components (control point 3, 4, & 5), or through alteration of the rate or path of ingress of external water into the device (control point 6).

FIG. 2: Adjustment of Elution Kinetics through the use of kinetics modifiers. Lidocaine freebase elution kinetics are altered by varying alpha-tocopherol acetate content (0, 5, 10 15, 20 & 29% by weight) within a specific carrier. All formulations were prepared with 16% Lidocaine free base. See Tables 4.1 & 4.4 for details. (A) Time course of cumulative % Lidocaine eluted. (B) Time course of change in Lidocaine release rate from the same formulations. In both panels, the curves vary systematically with tocopherol content with the lower curve representing the highest tocopherol content, and the upper curve representing 0% tocopherol.

FIG. 3: Effect of fatty acid chain length on absorption rate in the sheep tibial defect model. Product absorption was scored on a 3 point scale: 0=no absorption; 2=complete absorption. (A) Calcium Stearate 55%; TA 5%; PLU 24%; LFB 16%; (B) Calcium Stearate 50%; TA 5%; PLU35 14.5%; PLU68 14.5%; LFB 16; (C) Calcium Laurate 67%; TA 5%; TEC 12%; LFB 16%; (D) Calcium Laurate 62.5%; TA 7.5%; PLU35 14%; LFB 16%.

FIG. 4: Adjusting absorption rate using dispersing agents. Shown are the in vivo absorption results at four weeks in the sheep tibial defect model. (A) Control: 55% calcium stearate, 24% Pluronic L-35, 5% TOC acetate; (B) 31% calcium stearate, 31% GPCS, 17% Triethyl Citrate, 5% TOC acetate; (C) 45% calcium stearate, 18% Phosphatydil-choline, 19% Pluronic L-35, 5% TOC acetate; (D) 25% calcium stearate, 35% GPCS, 15% Pluronic L-35, 10% TOC acetate. All formulations contained 16% lidocaine free base. Product absorption was scored on a 3 point scale: 0=no absorption; 2=complete absorption.

FIG. 5: Adjusting absorption rate using water soluble solids. Shown are the in vivo absorption results at four weeks in the sheep tibial defect model. (A) Control: 55% calcium stearate, 24% Pluronic L-35, 5% TOC acetate; (B) 45% tricalcium phosphate, 8% PEG 2000, 16% PEG 900, 10% TOC acetate; (C) 13% calcium stearate, 62% PEG 2000, 11% TOC acetate; (D) 40% tricalcium phosphate, 10% calcium stearate, 19% Pluronic F-68, 10% Pluronic P-123, 5% TOC acetate. All formulations contained 16% lidocaine free base, except (C) which contained 14%. Product absorption was scored on a 3 point scale: 0=no absorption; 2=complete absorption.

FIG. 6: In vivo absorption and bone healing. Results are shown at four weeks in the sheep tibial slot defect model. Formulations A-S are given in Table 8.2. TCP control is B-TCP from Berkeley Advanced Biomaterials, Inc. Product absorption was scored on a 3 point scale: 0=no absorption; 2=complete absorption. Bone healing was scored on a 3 point scale: 0=no bone growth; 2=complete filling of the defect with newly formed bone. In each pair of bars, the bar on the left represents putty absorption and the bar on the right represents bone healing.

FIG. 7: In vivo elution of lovastatin. Shown is the amount of Lovastatin remaining in samples (mg/mg putty) over a 67-hour period. Lovastatin concentrations were determined spectrophotometrically.

FIG. 8: Lidocaine delivery as a function of hydrophilic to lipophilic balance (HLB). Pluronics used are listed in Table 20.2.

FIG. 9: Elution rates. Diamonds, 40% CaP, 10% CS, 19% F-68, 10% P-123, 5% TA, 16% LFB; Triangles, 40% CaP 8, 10% CS, 19% F-68, 10% P-123, 5% TA, 16% LFB; Stars, 40% CaP 16, 10% CS, 19% F-68, 10% P-123, 5% TA, 16% LFB;.

FIG. 10: Elution of lidocaine from osteoconductive carriers. (A) Percent (%) lidocaine released over time (hours). (B) Lidocaine release (mg/hr) over time (hours). The top lines represent the formulation: 40% TCP/10% CS/19% F-68/10% P-123/5% TA/16% LFP. The bottom lines represent Orthostat-L.

FIG. 11: Elution of lidocaine from ionic polymer formulations over time. Diamonds, SLAP; Squares, Orthostat+SLAP; Triangles, Xybrex+Lidocaine Alginate; “X” Xybrex.

### DETAILED DESCRIPTION OF THE INVENTION

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

“Agent” refers to a substance to be delivered to the body in a site specific manner. Generally agents will have a medical or commercial use. Most often the agents will be biologically active and/or have a therapeutic function once present in the body. In some embodiments, agents exist within the composition of the invention in depot form. That is they may be present as a precursor, salt, ion pair, or complexed with a molecular chaperone or carrier. In general the agent represents the biologically active form, which may be generated at any point between the carrier device and the point of action of the agent.

“Agent Depot” is an over-arching term referring to the form or formulation of the agent within the device. Specifically, the agent may exist alone, as a precursor, or complexed with carriers (e.g. proteins) or as a salt in order to affect the stability, storage, release and/or transport of the agent. In many embodiments, multiple forms of the Agent Depot (AD) exist in the same device (e.g. two or more salt forms).

“Aqueous dissolution” when used in reference to the AD is the solvation of the AD directly from the device into the aqueous milieu. Aqueous dissolution may occur by two specific mechanisms: The AD is present in the device in a solid form and dissolves directly in to the aqueous extracellular milieu. Or the AD is present within the device in solution in a non aqueous liquid medium which interfaces the aqueous milieu and the AD partitions from the liquid across the interface into the aqueous milieu.

“Capacity” refers to the amount of agent or AD capable of being incorporated within the vehicle.

“Coordinate dissolution” refers to mobilization of the AD from the device requiring the solvation action of one or more components of the mobile phase.

“Delivery kinetics” refers to the time course of delivery of the agent from the vehicle. The delivery kinetics of a therapeutic agent or mobile phase from the micronized products are defined by the amount of material diffusing from the device over time. A variety of approaches may be taken to describe the delivery rate of the material or agent including the rate of release per unit time, the rate of release per cm² of the device surface area, or cumulative release, etc. The delivery kinetics of any of the inventive devices can be considered to have a first phase (first kinetic phase) followed by one or more additional phases (additional kinetic phases). The phases of release may be defined quantitatively according to the slope of the elution kinetics, for instance as determined by in vitro elution studies, or alternatively according the elution of a mobile phase which has a role in determining the rate of AD elution.

“Initial Release Phase” The initial phase of the kinetics of AD delivery can generally be approximated by a single mathematical expression and is the time period when up to 70% or more of the therapeutic agent and/or the mobile phase is eluted. In preferred embodiments, elution of up to 60% of the mobile phase and/or therapeutic agent occurs in the initial release phase. In many embodiments less than 50%, less than 40%, less than 30% or less than 20% of the therapeutic agent or mobile phase is eluted during the initial release phase. In those instances where the delivery kinetics are defined by a burst phase, the initial release phase will generally comprise most or all of the burst phase.

“Additional kinetic phase” Additional kinetic phases for the delivery of the AD from the device may also be defined. In aggregate, all kinetic phases other than the initial kinetic phase are considered to comprise the “sustained release phase”.

“Impermanent mobile phase” The mobile phase of the invention is impermanent in as much as it is capable of diffusing from the device.

“Limited solubility” when used in reference to the solubility of the solid carrier in the liquid vehicle, means the solubility is such that dissolution of the carrier into the vehicle under the conditions defined (eg during storage), occurs minimally, such that the majority of the carrier remains undissovled, generally this means more than 70%, preferably greater than 80, 90 or 99%. Limited solubility can also refer to the carrier in certain carrier/vehicle systems where the carrier may have considerable solubility within the vehicle but the vehicle is pre-saturated with carrier material prior to introducing the carrier in solid form to the vehicle, therby ensuring retention of the carrier in solid form.

“Liquid vehicle” or “vehicle” refers specifically to liquid forms of the mobile phase which are combined with the solid phase to produce the carrier system.

“Malleable solid” refers to solids that are moldable at room or body temperature as exemplified by waxes and soft waxes such as paraffin and polyethylene glycol, and suspensions of micronized particles within the mobile phase.

“Micronized solid phase” is a granular solid phase that in most instances represents the principle volumetric component of the composition of the invention. The micronized solid phase is distinguished from a hydrogel in that it is less than 90% H₂O, and most often contains little or no water. The micronized solids of the invention are distinguished from liquids (e.g. viscous oils, aqueous solutions etc.) but not necessarily flowable or moldable waxes. For the purpose of this application, micronizable malleable solids, waxes and waxy materials are potentially micronized solids (e.g. frozen or pulverized). In general, the solid phases of the invention are matched with their mobile phases such that they are not solubilized by and are dispersible within, the mobile phase. Some solid carriers of the invention are not micronized ie, they are formable waxes or polymers.

“Mobile phase” is defined as the part of the device which most rapidly exits the device following implantation into the body. The mobile phase is comprised of one or more compounds, and is considered to be either simple or compound depending upon the kinetics and/or mechanism of its mobilization from the device. Simple mobile phases exhibit single phase elution kinetics, compound mobile phases exhibit more complex elution kinetics. The mobile phase generally has limited solubility within the aqueous milieu of the body (the extracellular fluids), or an otherwise extended solubilization time requiring hours or days to fully exit the device. The mobile phase is most often an organic liquid such as liquid Poloxamers, aliphatic alcohols, polyethylene glycols or organic acids. In the case of liquid mobile phases, the mobile phase may represent all or a part of the total amount of liquid present in the device. Solid mobile phases are also possible and are characterized by moldability when combined with the micronized solid carriers of the invention. Solid surfactants, and particularly solid neutral surfactants such as Pluronics, polyethylene glycols and pegylated derivatives of tocopherol with melting points slightly greater than room temperature are exemplary solid mobile phases. In some instances, such as in the case of liquid anesthetics such as fluorine or halothane, the AD itself may be part or all of the mobile phase.

“Particulate” or “particle” may pertain to any solid material in crystalline or amorphous form. The particulate may be a single compound or a mixture of compounds. While particles may range in shape from spherical to irregular, including plate-like and rod-like, in their largest dimension particles are generally less than 500 microns, preferably less than 100 microns and most preferably less than 50 microns. Many preferred compositions of the invention comprise a substantial proportion of particles being less than 25 microns in their largest dimensions. Nanoparticles are useful in many embodiments of the invention. “Particulate” in the context of the invention explicitly excludes liposomes and micelles as well as other similarly configured two phase or multiphase phase systems comprising water encapsulated by or enclosed in a lipid boundary layer.

Some nanoparticulates of the invention may be fabricated by nano or micromolding techniques, such as those described by the Liquidia corporation in pending U.S. patent application publications, 20110306878, 20110300293, 20110123446, 20100216928, 20100190654, 20100173113, 20100055459, 20100003291, 20090266415, 20090250588, 20090131959, 20080251976, 20080131692, and 20070178133, incorporated herein by reference. In one preferred embodiment the carriers are molded containing inclusions such as ceramics, drug-containing ceramics, calcium phosphates, drugs, or other erodible materials.

“Reservoir” is a device component the residence time of which within the device is prolonged following implantation (at least 2, preferably longer than 3 days) into the aqueous milieu, and the presence of which prolongs the residence of the AD in the device. Tocopherol acetate, low solubility surfactants with HLBs less than 10) (eg poloxamers such as Pluronic L-121 and L-L-101, L-81, L-61, and L-31), liquid polypropylene oxides and or polypropylene glycols, cholesterols, and SAIB as they are used in many formulations herein are exemplary reservoirs. In some preferred embodiments the reservoir specifically contains no tocopherol or tocopherol components.

“Single phase liquid vehicle” refers to a liquid or mobile phase comprising one or more liquids and solutes but which exists as a single uniform phase as distinguished from a liquid comprising, an aqueous and an organic phase, different density phases, or liquid containing micelles, liposomes or sol gel systems.

“Therapeutic Delivery Rate” or “Therapeutic Release Rate” is an AD rate of release from the inventive devices that provides a therapeutic effect.

“Transitioning Mobile Phase.” Typical mechanisms for the mobilization of the therapeutic agent from the inventive device include: aqueous dissolution, coordinate dissolution and partitioning. Complex mobile phases have been designed in which the mobile phase of the device undergoes a change in chemical character as it and/or its components gradually dissolve away from the device, in many cases leaving the residual mobile phase with a continually increasing character of the reservoir. This Transition in character of the mobile phase may have a direct impact on any or all of the three AD mobilization mechanisms. Specifically, the solubilization and depletion of all or part of the mobile phase (components) leads to a transitional shift in the mechanism of subsequent AD mobilization & release from the device.

The starting condition for the mobilization mechanism underlying AD release from the device can be referred to as Mechanism A (A). The final mobilization condition can be referred to as Mechanism B (B).

The shift in mobilization mechanism occurring as the mobile phase dissolves over the lifespan of the device can be summarized as:

### A→Intermediate→B

Where “intermediate” is generally a combination of both mechanisms A & B.

“Transport Properties” refers to the drug delivery kinetics and their underlying mechanisms with respect to the inventive devices.

“Tunable” refers to the ability to adjust a given parameter according to need and desired outcome. For instance, many of the inventive devices have tunable initial and sustained kinetic release phases for their agent depots. The amount and actual kinetics of AD release during either of the phases may be adjusted or “tuned” according to the specific clinical need.

### Overview of the Invention.

The drug delivery devices described herein are implantable and locally deliver therapeutic agents to the implant site for extended periods of time. The devices deliver therapeutic agents passively through diffusion directly into cells, tissues or the aqueous milieu at the site where they are implanted, and in many cases feature a micronized solid carrier, a compound mobile phase, comprising a reservoir, and at least one therapeutic agent referred to herein as an agent depot. The components of the device are mixed to produce a malleable putty, paste or lotion which may then be implanted to a specific target site within the body in need of the therapeutic agent. In some circumstances the inventive compositions are also useful topically for transdermal AD delivery or for the treatment of wounds or burns.

The inventive drug delivery devices are distinguished by unique relationships between burst and sustained release phase kinetics of the AD such that the burst phases produced are of lower magnitude and often persist for greater periods of time relative to the sustained release phase than previously thought possible for liquid based delivery systems. The delivery devices described herein are also differentiated by 1.) prolonged release of the AD at therapeutically useful rates. 2.) complex tunable delivery kinetics not easily defined by simple exponential or linear equations. 3.) the ability to alter in vivo resorption time while maintaining specific agent delivery kinetics, 4.) their resistance to water and dispersion, and 5.) in many instances, the substantial anhydrous nature of their formulations.

To understand the operation of the inventive devices, the nature and role of the three functional components of the drug delivery system need to be appreciated. They are 1.) the micronized solid phase, 2.) the mobile phase and 3.) the therapeutic agent or agent depot, wherein the micronized solid phase and all or a portion of the mobile phase represent the carrier system.

### The Carrier System

The carrier system refers to the combination of mobile and micronized solid phases without consideration of a therapeutic agent such as drug. The carrier system is generally comprised of a biocompatible granular solid phase with at least partial hydrophobic character and a mobile phase less polar than water or with at least partial hydrophobic character. When the carrier is placed in an aqueous environment, the mobile phase remains associated with the solid phase for a period of time. Many of the device components useful in this invention are similar to those described by U.S. Patent Publication Nos. 2005/0065214; 2006/0002976; and 2006/0280801, incorporated herein by reference in their entireties. In vitro elution from the inventive devices of drugs such as lidocaine is described in example 1.

The Mobile Phase:

The mobile phase is generally anhydrous or substantially anhydrous. Part or all of the mobile phase is water soluble and dissolves slowly from the device into the aqueous milieu of the body over an extended period of time. Mobile phase composition and solubility contribute to the key features underlying the complex tunable delivery kinetics of the syntinate device. Differential escape of mobile phase components from the device into the aqueous milieu of the body can be used to produce a steady transition of the drug transport properties within the device. Transport properties transition from an initial state at the time of implantation to a final state when the last of the drug is eluted. The inventive devices generally employ mobile phases with at least two components; a first long residence time, largely water insoluble liquid referred to as the reservoir component and a second poorly water soluble, most often amphiphilic component referred to as the kinetics modifying component. The reservoir component when used as a unitary mobile phase with the micronized solid, releases the AD slowly or not at all, and establishes the basal delivery rate for the AD which can be accelerated by the addition of a kinetics modifier. When used in conjunction with a kinetics modifier, the reservoir component significantly impacts the final transport properties or sustained release phase of the device, while the kinetics modifier generally increases the kinetic rates for burst and intermediate time points.

Following implantation within the body of a recipient, all of the mobile phase is retained as a part of the carrier for an extended period of time due to either or both of: a.) its solubility limitations within the extracellular fluid environment, b.) its affinity for or partitioning into the solid phase (e.g. through hydrophobic interaction, Van der Waals forces or other affinity or adsorption means). Mechanism notwithstanding, the mobile phase is retained at the implant site for a period of time appropriate to the desired rate of release of the Agent Depot. The mobile phase may comprise a single component or be a solution or suspension of multiple components. The mobile phase serves one or more of five purposes: 1.) to promote suspension of the solid phase and provide lubricity to the carrier complex, 2.) to shield part or all of the agent depot from direct contact with the aqueous environment for a desired amount of time, 3.) to dissolve, disperse or suspend the AD, 4.) to deliver some or all of the therapeutic agent to the aqueous milieu following implantation, and 5.) to provide complex delivery kinetics to reduce burst and/or enhance the sustained release phase.

In many instances the use of hydrophobic substituents within the mobile phase will also provide a valuable contribution to the overall water resistance of the device. Hydrophobic moieties may also serve to shield the AD from dissolution into the aqueous environment while dissolved within the mobile phase, or in the case of an AD insoluble within the mobile phase, dispersion therein may also shield the AD from aqueous dissolution.

In some embodiments the AD is soluble within the mobile phase and departs the device along with the mobile phase. In other embodiments the agent or agent depot remains either completely or partially un-dissolved within the mobile phase, and part or all of the mobile phase serves as a covering or barrier between the agent and the aqueous environment. Following placement of the device within an aqueous environment, the length of time part or all of the AD is shielded from the aqueous environment, by a component of the mobile phase, is a significant parameter defining the specific release kinetics of the AD from the device.

In most embodiments, the mobile phase has at least partial hydrophobic character or possess another property such that it has an affinity for the solid phase (e.g. through a hydrophobic interaction) such that the mobile phase does not freely diffuse away from the solid phase in an aqueous environment. In fact the mobile phase and corresponding solid phase are chosen such that depending upon the desired rate of delivery of the AD to the body, the mobile phase/solid phase interaction remains stable over the course of hours, days, weeks or months. In many preferred instances the mobile phase is comprised of multiple components (compound mobile phase) which undergo solubilization into the milieu surrounding the implant site at different rates. In these embodiments, each mobile phase component generally remains associated with at least some of the AD, such that the differential solubilization of the multiple components leads to different phases of therapeutic agent release from the device. Compound mobile phases which perform in this manner lead to more complex delivery kinetics of the AD than might occur with a single component mobile phase.

Exemplary mobile phases comprise one or more of a surfactant (e.g. liquid or solid poloxamer), a poorly water soluble or insoluble oil, a fatty acid alcohol, a liquid reducing agent such as tocopherol and/or its derivatives, a vitamin K and/or derivatives, SAIB and derivatives thereof. The preferred mobile phases comprise one or more substituents with Log P of greater than 2, most preferably greater than 3 and most preferably greater than 5. In the most preferred mobile phases, one or more of the constituents will create a two phase, oil and water system with water, with the therapeutic agent partitioning into the oil phase in a ratio exceeding twice and preferably exceeding four times that of its portioning into water.

Reservoir Component of the Mobile Phase.

In most embodiments, the mobile phase will comprise a slowly water soluble or water insoluble organic liquid or solid, referred to as the reservoir component. The reservoir component is capable of solublizing, suspending or shielding the AD, without solubilizing the micronized solid. Typical reservoir components are pure or substantially pure, poorly water soluble or insoluble oils generally capable of forming two phase systems with water. Exemplary reservoirs include sucrose acetate isobutyrate (SAIB), alpha-tocopherol acetate, vitamin K1, cholesterol, steroidal compounds, fatty esters, α-Tocopherol, pegylated tocopherol succinate, sorbitan oleate, sorbitan laurate, and derivatives and modifications thereof. Additional reservoir candidates will be known to practitioners, many of which are listed by McCutcheon's (2009)). Some poorly soluble liquid neutral surfactants such as PLURONIC L-121, PLURONIC L-122, and PLURONIC L-101, or compounds with similar characteristics are also suitable reservoirs. Suitable reservoirs release AD solubilized within them either slowly or not at all for 48 hours, preferably 72 hours or longer. In test two phase partitioning systems employing the reservoir and water, the AD preferentially partitions into the reservoir phase at a rate of greater than 52:48 (wt/wt) preferably greater than 60:40, and most preferably great than 80:20. Many reservoirs will be selected because the AD partitioning of the AD between the reservoir candidate and water is greater than 95:5. Reservoir candidates may be screened by preparing a putty by combining the reservoir with calcium stearate and 16% lidocaine free base as described in Example 1 among others. Lidocaine elution from the putty can then be tested according to Example 1. When tested for lidocaine elution, suitable reservoirs will release less than 80% of the lidocaine content within 72 hours, preferably less than 50% and most preferably less than 25%. At the 72 hour time point lidocaine release from a suitable reservoir will be greater than 0.2 mg/hr, preferably greater than 0.5 mg/hr and most preferably greater than 0.8 gm/hr. Furthermore the initial lidocaine release rate should diminish to less than or equal to 11.8 mg/hr within 45 hours, preferably within ≦40 hrs, ≦34, ≦20, or ≦12 hours.

Kinetics Modifier.

In preferred embodiments at least one liquid or solubilized surfactant, detergent or other surface active compound useful for dispersing oil in water or water in oil is present within the mobile phase. Particularly useful liquid surfactants include:

Liquid poloxamers, nonanol, tween compounds such as tween 80, phospholipids, sorbitan, sorbitan esters, including sorbitan oxalate, sorbitan laurate and sorbitan stearate, glycerol monostearate, 12 hydroxy stearic acid, triton and Brij surfactants, nonionic detergents and surfactants, emulsifying surfactants with HLB numbers between 0.5 and 50, preferably between 1 and 25 and most preferably less than 20, 19, 18 or 17; compounds with surface tensions similar to any of the liquid Pluronics with HLB numbers in the range of 1 to 30, and surfactants with critical micelle concentrations in the range of 0.0002 to 1% preferably 0.0008 to 0.9%. Particularly useful are nonionic, liquid Pluronics with an HLB value of less than 19 and a CMC value less than 1%, preferably less than 0.14% and in some cases between 0.0004% to 0.0008%. “Exemplary compounds and surfactants available in forms with a range of HLBs of 17 or less are commercially available under brand names such as SPAN, BRIJ, MYRJ, TWEEN, LIPOSORB, TERGITOL, LIPOCOL, PLURONIC, TRITON, CANASOL, CREMOPHOR, LIPOMULSE, LIPOPEG, Generic materials with HLBs less than 17 include derivatives and modifications of Sorbitan, polyacrylates, alkoxylated fatty alcohols, random and non-random block copolymers of polypropylene oxide and ethylene oxide, ethylene glycol derivatives, propylene glycols, blocked polymers (e.g., Chemal BP 261), silicon glycol co-polymers, polyoxyethylene ethers, ethoxylated triglycerides, ethoxylated fatty acids, and ethoxylated fatty amines. Practitioners in the art will recognize many other liquid surfactants with similar properties are available and can be identified in publications know to the art such as McCutcheon's Volumes 1 & 2 (2009) and Batrakova et al., (2003), incorporated herein by reference.

Surfactants existing as solids or waxes may also be employed as kinetics modifiers. These materials may be used either as solids, or be solubilized in another organic liquid. Exemplary waxy and mallelable solid surfactants useful in the mobile phase include: polyethylene glycols, solid poloxamers (eg Pluronics F68 & P123, P85 & 25R4), phospholipids such as phosphatidic acid, phosphatidylcholine, and phosphatidyl serine, and amphiphilic tocopherol compounds including tocopherol succinate, and tocopherol PEG succinate. Solid kinetics modifiers are often melted in order to fabricate homogeneous mixtures with the micronized solid, the AD and the reservoir component. Preferred kinetics modifiers may be used to produce delivery systems which when tested for delivery parameters with Lidocaine produce a range of kinetic parameters as described in the examples.

Other Useful Mobile Phase Constituents:

Other potential components of all or part of the mobile phase include: triethyl citrate, liquid and/or eutectic salts of therapeutic agents, and liquid therapeutic agents.

The Solid Phase

The solid phase is most often comprised of a finely powdered (micronized) water resistant matrix. In most of the inventive drug delivery systems, the solid phase comprises a pure, substantially water insoluble, organic, GRAS (generally regarded safe) solid capable of being metabolized by the body.

The primary role of the solid phase is to ensure the device maintains its position at the implant site and to establish the physical properties of the device. The chemical identity of the solid phase, as well as its particle size and distribution may also have a significant effect on one or more device properties, including: 1.) water resistance, 2.) handling properties, 3.) residence time at the implant site, including in vivo absorption and/or dispersion characteristics, 4.) the time course of disappearance of the mobile phase, and 5.) the delivery kinetics of the AD. 6.) establishing and maintaining a diffusion distance (diffusion path) for the AD, 7.) enhanced sustained release phase for long term AD delivery, and 8.) hemostatic tamponade or other bulking capabilities if required.

In some embodiments the AD itself may adsorb, adhere or otherwise have affinity to the solid phase. In other embodiments of the invention, the micronized solid phase is also constructed to contain one or more embedded therapeutic agents which may be released following implantation during the resorption and/or dissolution of the micronized solid phase. Typically the micronized solid phase is carefully matched to the mobile phase to provide the optimal delivery profile of the agent or agent depot, and in most instances the micronized solid phase is insoluble or minimally soluble within the mobile phase.

The performance requirements of the specific application will dictate the final properties required for the micronized solid phase. Generally speaking however, all or most of the micronized solid phase of the composition will need to remain in place in situ until delivery of the desired drug is completed. For this reason, the time for complete dissolution/dispersion/metabolism of the micronized solid phase will generally need to exceed the required time of delivery of the agent. Additionally, it is often advantageous to use a water resistant solid to help establish the water resistant properties of the final device, although water resistance can also be established or enhanced through the use of appropriate water resistant mobile phase constituents (e.g. Tocopherol and derivatives, SAIB or low solubility liquid Pluronics) present either alone or dissolved within one or more device components.

Properties of the Solid Phase

Composition:

The micronized solid phase is comprised of one or more materials. Preferred micronized solid phases include metal salts of fatty acids and their derivatives. Kronenthal 2004 incorporated herein by reference, presents a partial list of suitable micronized carriers. Other suitable materials for use in the micronized solid phase include cholesterol, cholesterol derivatives and modifications, solid polyethylene glycols and their derivatives as well as fatty acid salts of therapeutic agents. Calcium salts of phospholipids are also useful as micronized carriers. When using metal salts of fatty acids as the primary component of the micronized solid phase it has been found that other water soluble salts may be added up to about 20% by weight and sometimes even up to 30%.

Particle Size:

In general, particle size will affect the cohesiveness, water resistance and flowability of the putty. The solid phase comprises micronized particles of less than 300 micron diameter preferably less than 250 and most often less than 100 or 50 microns. Many preferred embodiments employ particle sizes less than 10 microns. Nanoparticles are also contemplated in many of the inventive compositions. In addition to micronized particles, the solid phase may also contain larger particles such as particles of osteoconductive, osteoinductive, water absorptive, hemostatic agents, solid particles of the agent or agent depot, drug immobilizing particles or matrices (e.g. oxidized cellulose, carboxy methyl cellulose, starch, modified starch, calcium phosphates, hydroxyapaptite (HA), HA/TCP, glasses and bioglasses, and silicate calcium phosphates). These particles themselves may be micronized or in some instances may have particle sizes in larger than three hundred microns, sometimes larger than 500 or 1000 microns. In some embodiments the additional particles in fiber form, will be present as whiskers or larger. Fibrillar particles will have lengths exceeding their smallest diameter by two fold and most often the lengths will exceed the smallest dimension by five or ten fold. Such fibrillar particles may be included to promote cohesiveness, tensile strength or specific biological properties, as described by Knaack et al. U.S. Pat. No. 6,500,516; Winterbottom et al. WO20050251267, both of which are incorporated herein by reference, in their entireties).

### Hydrophobic Character and Water Resistance of the Solid Phase.

Cholesterol and cholesterol derivatives, SAIB and metal salts of alkyl organic compounds such as fatty acids and phospholipids, and more specifically, divalent metal salts such as Ca²⁺, Zn²⁺, Ag²⁺ and Mg²⁺ salts of laurate, palmitate, stearate, and phosphatidic acid when suspended in an appropriate surfactant or hydrophobic liquid, have been found to impart excellent water resistivity to the inventive devices. Salts of other divalent compounds such as di-, tri- and oligo-peptides (e.g. poly-lysines or poly-arginines) or other divalent cationic compounds capable of linking multiple alkyl organic molecules and reducing their solubility or making them insoluble are also contemplated. Preferred formulations prepared with alkyl salts have been demonstrated to remain cohesive in aqueous environments for extended periods of time, and can resist manipulation under water, or the flow of aqueous irrigants such as saline and pure water. In some preferred embodiments enzymatically susceptible di- or multi-valent cationic materials (e.g. poly lysine) may be employed as linking agents to fatty acids or other appropriate ionic molecules to create an insoluble complex which may be enzymatically cleaved and solubilized after implantation into a living organism.

When using solids for which the water resistance properties of the solid alone is not satisfactory, water resistance may be improved by addition up to 20% calcium stearate or other metal salt of a fatty acid, or alternatively by using a viscous hydrophobic additive in the mobile phase such as a tocopherol compound, SAIB or a water insoluble surfactant. Malleable waxy solids such as solid poloxamers, cholesterol, polyethylene glycols, phospholipids or derivatives or modifications thereof as well as thickeners known to the art (cf Mcutcheon, 2009) may also be added to the formulation to improve water resistance.

Other micronized solid phase components used with the compositions of the invention include insoluble acyl glycerols and glycerol phosphates, absorbable polymers including lactides, galactides and tyrosine polycarbonates, tyrosine polyarylates, absorbable polyurethanes (B. Li et al./Biomaterials 30 (2009) 3486-3494), fumerates, insoluble Pluronics (poloxamers), pegylated protein-based polymers, polyethylene glycols, particulate ceramics such as calcium phosphates, magnesium phosphates, calcium sulfates, phosphate glasses, silicates and bioglasses. In some instances, the solid carrier will carry an ionic charge, one or more double bonds, or be capable of hydrogen bonding (e.g. through free hydroxyl groups). Compositions of the invention can also have solid phase components comprising some or all of the substances listed above in varying proportions.

In one important embodiment biphasic particulates are employed. Specifically, hydrophilic particles such as a calcium phosphate granules are embedded within a hydrophobic matrix to produce the micronized solid (Masafumi Uota, et al., (2005) Synthesis of High Surface Area HydroxyapatiteNanoparticles by Mixed Surfactant-Mediated Approach. Langmuir 21, 4724-4728).

### The Agent Depot

The agent depot comprises the therapeutic agent to be delivered. In many embodiments the agent or therapeutic drug is the agent depot. In other embodiments, the agent is modified or complexed with other components to produce an agent depot having the properties needed in order to achieve a desirable result. The pairing of mobile phase and agent depot is made such that the stability of the agent depot, the delivery kinetics of the agent depot following implantation, and/or the interaction of the mobile phase with the agent depot is stable over the course of hours, days, weeks or months, depending upon the desired rate of delivery of the agent or agent complex to the body. The agent depot is generally soluble within all or part of the mobile and/or micronized solid phase, and following implantation of the composition of the invention, the agent or agent depot is released from the composition into the extracellular milieu according to predetermined kinetics. The mobile phase isolates part or all, of the total amount of the agent depot from the aqueous environment and thereby controls the availability of the agent depot to the environment.

Preferred agent depots (ADs) of the instant invention will most often be organic molecules with significant hydrophobic character. The agents will be chosen first because they meet a specific therapeutic need. Preferred agent depots will be capable of being matched with a mobile phase such that delivery of the therapeutic agent will occur in vivo over a rate compatible with the therapeutic need.

Generally the AD will at least be partially, if not completely soluble, within the mobile phase. In some cases where the agent is not soluble within the chosen mobile phase the mobile phase may be heated to promote solubility of the AD. This may be particularly important to ensure homogenous distribution of the AD within the mobile phase in those instances where the mobile phase is a wax or a solid. During formulation, the mobile phase may be heated to promote solubility of the agent depot. Systems in which the AD preferentially partitions into the mobile phase as compared to the aqueous environment are often preferred A table of partition coefficients (log P) (eg handbook of Chemistry and Physics 2006) may be consulted to select forms of the AD which preferentially partition into organic environments or into specific components of the mobile phase.

Simple modifications of the AD to improve or otherwise adjust compatibility with the mobile phase while preserving potency are often possible. Such modifications include producing the AD in alternative salt forms, employing ionic interaction with charged moieties, or lyophilization, or complexation. In some embodiments water soluble ADs are prepared dry, as in the case of a lyophilized protein and suspended within a mobile phase with which it is compatible. Glycerol or other polyol molecular chaperone-like molecules may also be used to stabilize the protein in the delivery system.

A simple preliminary test for compatibility of the AD with the mobile phase is to mix one or more forms of the AD with potential mobile phase candidates, retrieve the AD and test for potency in an appropriate assay system. A series of potential mobile phases may be screened for compatibility with the AD. As a preliminary formulation step it is preferred to identify 3-5 potential mobile phases that are compatible with the AD.

Highly water soluble agent depots may also be delivered by the inventive devices. These include sugars, salts, nucleic acid and protein agent depots. Lyophilized proteins and dry powders are administered to subjects by the compositions of the invention through anhydrous delivery. Agents delivered in this manner include proteins, nucleic acid agents, glycosaminoglycans, proteoglycans and glycoproteins.

Hydrophobic agents which occur in both the acid and free base form may be derivatized using standard acid base chemistry known to the art. The free bases may often be reacted with acids or salts to produce salt forms which may have improved compatibility or stability within a specific mobile phase. Thus free base forms of local anesthetics may be reacted with fatty acids or other organic molecules containing free carboxylic acid moieties

Preferred agents and agent depots include salts, ion pairs or chemical derivitizations of the compounds listed below.

In some embodiments, the agent used with the devices and compositions of the invention is a statin. Statins include atorvastatin, cerivastatin, fluvastatin, lovastatin, mevastatin, pitavastatin, pravastatin, rosuvastatin, simvastatin, simvastatin with ezetimibe, lovastatin with niacin, and atorvastatin with amlodipine besylate. In addition to statins which may be delivered to promote bone growth or effect lipid synthesis, a wide variety of ADs capable of affecting bone mineral density, or bone metabolism are also contemplated. These include: Vitamin D, calcitonin, serotonin, serotonin uptake inhibitors, insulin and insulin like growth factors BMP, calcitriol, calcidiol, growth hormone, PTH (teraparatide), sodium fluoride, PDGF, prostaglandin E1, bisphosphonates. In other embodiments, the agent used with the devices and compositions of the invention is an anti-inflammatory. Anti-inflammatories include corticosteroids and glucocorticosteroids. Specific examples of anti-inflammatories include cromoglicate, nedocromil, salmeterol, flunisolide, mometasone furoate, triamcinolone, fluticasone, budesonide, formoterol, beclometasone dipropionate, zileuton, MK-886, montelukast, and zafirlukast.

In other embodiments, the agent used with the devices and compositions of the invention is an anticancer therapeutic. Anticancer therapeutics include cisplatin, carboplatin, mechlorethamine, cyclophosphamide, chlorambucil, azathioprine, mercaptopurine, vinca alkaloids, taxanes, vincristine, vinblastine, vinorelbine, vindesine, podophyllotoxin, etoposide, teniposide, dactinomycin, dexamethasone and finasteride.

In other embodiments, the agent used with the devices and compositions of the invention is an anesthetic. The anesthetic may be in an acid, free base, or salt form. Of particular value are the lipid salts of anesthetics, since the choice of the lipid salt form may be manipulated to influence, the anesthetic, stability or elution properties as desired. Useful anesthetics include procaine, tetracaine, amethocaine, cocaine, lidocaine, prilocalne, bupivacaine, levobupivacaine, ropivacaine, dibucaine, thiopental, methohexital, midazolam, lorazepam, diazepam, propofol, etomidate, ketamine, fentanyl, alfentanil, sufentanil, remifentanil, buprenorphine, butorphanol, diamorphine, hydromorphone, levorphanol, meperidine, methadone, morphine, nalbuphine, oxycodone, oxymorphone, flecanide, benzocaine, phenyloin, pentacaine, heptacaine, carbisocane, isoflurane, methoxyflurane, tocamidew, quinidine, mexiletine, alfaxalone, butamben, enflurane, sevoflurane and pentazocine, phenyloin, pentacaine, heptacaine, carbisocaine, isoflurane, methoxyflurane, tocanide, quinidine, mexilitine, alfaxalone, propofol, butamben, enflurane, sevoflurane. Preferred carboxylic acids for the use in production of anesthetic salts in an acid base reaction with an anesthetic freebase and that are particularly preferred for the local anesthetics Bupivicaine, Lidocaine Ropivicaine and Tetracaine, include Tocopherol PEG succinate, tocopherol succinate, aliphatic carboxylic acids and their derivatives. Also useful for making anesthetic salts are complex carbohydrates such as the cellulose carboxylates and their derivatives including carboxy methyl cellulose, oxidized cellulose, carboxymethyl cellulose, alginates, and hyaluronic acid.

Exemplary antimicrobials and antiseptics which may be included in the device include: beta lactams, cephalosporins, silver compounds, peptide antimicrobials, triclosan, gentamicin, tobramycin, silver, silver stearate and silver salts of fatty acids and lipids, ceftazidine, fluconozole, tetracycline, vancomycin, cephalexin, methicillin, gramicidin, minocycline and rifampin.

### Constructing the Delivery System

The instant invention describes the formulation of useful delivery systems particularly suited for the delivery of ADs that are hydrophobic or have significant hydrophobic character. In some instances, described below, non-hydrophobic ADs may also be delivered by the delivery systems of the invention. It is recommended for those compounds which are soluble in tocopherol acetate to begin by preparing initial formulations as described in Example 1 employing calcium stearate to establish baseline elution characteristics. Fine tuning may then be accomplished as described elsewhere herein.

In many embodiments, the delivery systems are formulated as follows:

1.) An agent, agent depot or agent complex with at least partial hydrophobic character that is appropriately miscible or soluble with the mobile phase is selected.

2.) Suitable long residence time reservoir candidates are selected. The suitable reservoir will have a substantial ability to either dissolve or coat the AD of choice, and will not detrimentally affect the stability of the AD.

3.) A micronized or partially micronized solid phase with limited solubility in both water and the mobile phase and preferably with substantial hydrophobic character (or reservoir), is selected. The micronized solid phase will most often be insoluble or poorly soluble in the mobile phase with which it is paired, as well as the physiological environment into which it is implanted. In all cases, the time of solubilization of the solid carrier in vivo will be consistent and require an amount of time in excess of the time required to deliver a desired therapeutic quantity of the agent

4.) A suitable test formulation putty is prepared by dissolving or blending the AD in the reservoir to form a mobile phase. Appropriate levels of heat may be applied to melt the mobile phase or AD as needed. In general, for ease of manufacture, the preferred form of the mobile phase is a liquid, but a homogenous solid is also acceptable. The mobile phase and the micronized solid phase are then mixed to homogeneity in a ratio to produce a suitable handling solid, putty, cream or ointment according to the needs of the intended use. In preferred embodiments the mixing of the mobile phase and the solid phase will be at least initiated while solution A is in liquid form.

5.) Determine baseline in vitro kinetics of release of the AD from the test formulation. Examples 1-3 describes a suitable in vitro test system in which the AD may be tested.

6.) Fine tune release characteristics through the introduction of a release rate modifier or by any of the other means described below (eg Example 4).

It is recommended for the first-time preparation of a delivery vehicle for a specific drug that a frame of reference pilot formulation be prepared containing (weight percent) 55% Calcium stearate and 45% tocopherol acetate as the reservoir/mobile phase in an appropriate ratio with the drug. Preferably the AD will be an organic molecule with solubility of >2% by weight in Tocopherol acetate.

The device may be manufactured aseptically or terminal sterilization may be used to ensure sterility of the final product. Because of the anhydrous nature of many of the inventive formulations, many therapeutic agents will have improved stability to gamma irradiation when incorporated therein. The use of free radical scavenger reducing agents such as tocopherol and its derivatives further improve the irradiation stability of many of the inventive formulations.

The above test formulations can be characterized relative to a reference formulation where a kinetics modifier comprising Pluronic L-35 has been added to achieve a final formulation of approximately (wt:wt:wt) 2.5:1:1 Pluronic L-35:tocopherol acetate:drug. One embodiment comprising Pluronic L-35, Tocopherol acetate and lidocaine has demonstrated to be stable to gamma irradiation doses of at least 30 KGy. Pluronic L-35 is uniquely suited for use in a test mobile phase because its HLB of 19 and CMC of 1% make it compatible with many potential ADs. Most often to extend delivery time of an AD over this test formulation, a surfactant with a lower HLB (<19) and/or a CMC (<1) than Pluronic L-35 will be employed.

The delivery system composition of the invention, including the test formulation described above is characterized for suitability using a series of performance tests. Examples of such tests are:

1.) The water resistance of the delivery system is confirmed.

2.) The viscosity of the delivery systems is evaluated.

3.) The elution kinetics of the therapeutic agent (i.e. the agent, agent depot or agent complex) of the compositions are determined in vitro.

4.) The stability of the agent, agent depot or agent complex within the formulation is confirmed.

5.) The delivery system composition is tested for in vivo performance properties using suitable in vivo efficacy models. These properties may include hemostasis, circulating drug levels, biocompatibility, bone healing, tissue healing and therapeutic effectiveness.

The use of the reference system described in Tables 4.1 and 4.4 with and without a kinetics modifier, reveals the relative effectiveness of the kinetics modifier in releasing the drug from the reservoir. Following each test, based on the results achieved, formulation adjustments may be made as described below.

Fine Tuning Performance Characteristics: Physical Properties

At any point in the testing series the formulation may be adjusted to achieve desired performance characteristics. Various techniques are used to tune the water resistance of the compositions of the invention. In general, formulations with minimally adequate water resistance are chosen in order to facilitate in vivo absorption. However, in those instances where prolonged (e.g. >48 hrs) in vivo delivery is desired it may be advantageous to select more highly water resistant formulations. Techniques include varying the liquid to solid phase ratio, reducing particle size, increasing or decreasing the use of hydrophobic solids, increasing or decreasing the use of aggregating liquids, and increasing or decreasing the use of viscous hydrophobic liquids. Increasing the amount of highly viscous water resistant liquids in the mobile phase such as tocopherol acetate, SAIB and other similar organic liquids is generally advantageous. Tocopherol acetate concentrations of greater than 5% weight, and preferably greater than 10% by weight have been shown to be particularly useful in increasing the water resistance of formulations containing divalent metal salts of fatty acids and/or liquid Pluronics. To acquire a faster absorbing fatty acid salt carrier, shorter length fatty acid salts are used. Preferably the salt used is calcium or magnesium. Other multivalent salts contemplated in the invention include zinc, iron, manganese, aluminum, lithium, copper, nickel, silver, and strontium. Shorter length fatty acids include palmitate and laurate. Also, unsaturated fatty acids, amides and esters, or alkaline calcium stearate can be used to acquire a faster absorbing fatty acid salt carrier.

In some situations selection of a Kinetics modifier with significant water solubility may lead to a surface slipperiness of the device when exposed to water. In many such circumstances surface slippery ness may be reduced by partial or complete replacement of the modifier with a less water soluble modifier. Example 20 provides formulations comprising low water solubility kinetics modifiers.

Other specific embodiments of the invention include compositions in which the micronized solid phase comprises the fatty acid calcium salt, and further comprises an osteoconductive dispersant and a composition comprising a soft tissue conductive dispersant.

Testing Water Resistance:

Significant water resistance is often a very important property to maintain continued drug delivery without premature washout from the inventive formulations. Quantitative tests for water resistance are easily devised. See Examples 2 and 3, for two such tests. These tests may be applied to the carrier system with or without the AD. Other tests may be constructed by spreading the composition of the invention on an immersible holder such as a screen, other porous structure or cup, immersing the holder and the putty in an aqueous bath and exposing the putty within the holder to a shear force produced by mixing, agitation or a flowing liquid, and determining the amount of time required to partially or fully dislodge the putty from the holder. Water resistant putties will require appreciably more time to dislodge than thick flour and water pastes, or the like. The formulation described in Example 2 (base carrier Table 2.1) produces a water resistant putty with excellent handling characteristics and serves quite well as a bone hemostat. The properties of this formulation serve as a good frame of reference for the development of other delivery devices.

Fine Tuning Performance Characteristics: Delivery Kinetics and In Vivo Absorption and/or Dispersion

Delivery Kinetics

Kinetics of AD delivery for any of the inventive devices can be considered to have a first phase (first kinetic phase) followed by one or more additional phases (additional kinetic phases). The phases of release may be defined quantitatively according to the slope of the elution kinetics, for instance as determined by in vitro AD elution studies, or alternatively according the elution of a mobile phase which has a role in determining the rate of AD elution.

Initial Kinetic Phase.

The initial delivery kinetics can generally be described by a single mathematical expression and is the period when up to 70% or more of the therapeutic agent and/or the mobile phase is eluted. In preferred embodiments, elution of up to 60% of the mobile phase and/or therapeutic agent is eluted during the initial phase. In many embodiments less than 50%, less than 40%, less than 30% or less than 20% of the therapeutic agent or mobile phase is eluted during the first kinetic phase. In those instances where the delivery kinetics are defined by a clearly demarcated burst phase, the first phase will generally comprise most or all of the burst phase.

Additional Kinetic Phases.

Additional kinetic phases for the delivery of the AD may also be defined for the delivery of the agent from the device. In aggregate, all kinetic phases other than the initial kinetic phase are considered to be a part of the sustained release phase.

Therapeutic Delivery Rate.

The inventive devices and formulations are designed to supply the therapeutic agent at a rate that is therapeutically useful for the intended device purpose. Therapeutically useful delivery rates for the AD maybe established from animal efficacy studies (cf Gandhi et al. 2008) potentially employing fluorescent or isotope labeled versions of the AD. Sometimes the required release rates may be obtained in or calculated from the literature. Gandhi et al. (2008), provide an example of the comparison of in vitro to in vivo release rates of the AD from the inventive devices to establish target in vitro delivery rates for formulation development. Examples 4a & 4b further demonstrate the relative in vitro release rates determined for lidocaine delivery devices. Once a target in vitro release rate corresponding to in vivo efficacy has been established, the device may be further fine-tuned to optimize the kinetics of AD release according to this target release rate. In general the goal of release rate optimization is to maintain the AD delivery rate above the relative therapeutic release rate target value for as long as required. In particularly preferred embodiments, a carrier formulation comprising micronized solid, a reservoir and an optional kinetics modifier, is mixed with an AD leading to release of AD greater than 1.8 mg/hr for greater than 40, preferably greater than 46 and most preferable greater than 50 hours.

Modifying Release Kinetics.

The delivery kinetics for a given mobile phase/solid phase/and therapeutic agent combination are prolonged by increasing the duration and/or amplitude of the sustained release phase. This may be accomplished by: a.) Delaying the elution of some of the AD from elution during the initial phase to elution during the sustained delivery phase. b.) Providing additional AD for delivery during the sustained release phase in order to increase the amplitude or duration of the sustained release phase. c.) Decreasing the duration of the initial phase. d.) In some cases a combination of a & b may be possible.

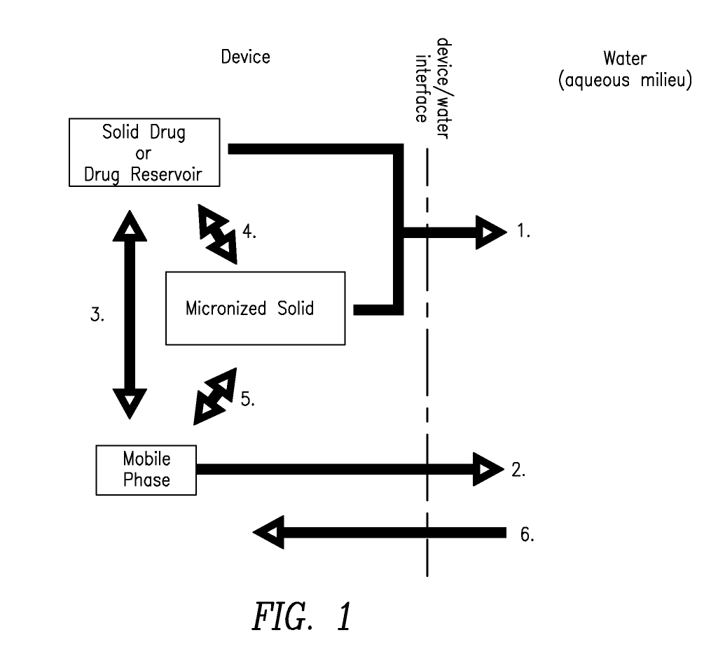

The unique composition of the invention and the interaction of its components offers the capability to fine tune the kinetics of AD delivery and prolongation of the period of therapeutically effective AD delivery. FIG. 1 depicts the possible AD release routes from the device (control points 1 & 2) and interactions of device components exploitable to alter the AD delivery kinetics. For all embodiments of the invention, AD is released from the device through one or both of two principle routes: The AD may be directly solubilized (control point 1) from the device into the aqueous milieu, and/or the AD may be carried from the device in conjunction with the solubilization of all or part of the mobile phase (control point 2).

The rate of release of the AD from the device is controlled through the direct manipulation of either or both of the release routes (control points 1 and 2), through manipulation of one or more interactions between the device components (control point 3, 4, & 5), or through alteration of the rate or path of ingress of external water into the device (control point 6). Procedures & examples pertaining to these approaches are described below.

Control Point 1: Direct Solubilization of the Drug from the Device.

In many cases the AD will exist within the device in a form which may be directly solubilized into the aqueous milieu surrounding the device. Directly solubilizable forms include: a.) AD in solid or reservoir form, and b.) Immobilized AD, where the AD is associated with a solid component as an embedded, or adsorbed form. In embodiments where the AD is soluble within a reservoir, the AD will often be directly solubilized into the aqueous milieu based upon its partitioning characteristics between the reservoir and the aqueous milieu. Prolongation of the release of the directly solubilizable AD may be accomplished according to the approaches described below.

Increasing Solid AD Content.

Particularly useful for extending the sustained release phase of ADs with limited aqueous solubility is the incorporation of additional solid AD within the device.

Reducing the Solubilization Rate of Solid AD.

Many strategies for the reduction of the solubilization rate of solid materials are available. Physical approaches include increasing particle size, crystallinity or packing density of the material, and/or embedding the AD with in a slowly solubilizing liquid or solid. Chemical alteration of the AD through the formation of salts, ion pairs or other chemical complexes, is also be useful to alter the rate of solubilization of the AD. In these cases the general rule is to produce a more hydrophobic form of the AD (see below for further details), or a less soluble or more slowly solubilizable complex (eg increased crystallinity). Through proper selection of an appropriate counter ion, such as an alkyl organic acid, more hydrophobic salts of the AD can be prepared. Counter ions may also be selected to produce salts with hydrogen bonding character; eutectic salts; and salts with varying solubilities. Methods for production of salts of drug substances are known to the art. Specific guidance may be found in Koyama (2005) and/or Serajudd (2007) incorporates herein by reference. Tables 4.7 & 4.8 also demonstrate the effect of using multiple salt forms of the AD. In some preferred embodiments a variety of salt forms with differing hydrophobic character of the anionic counter ion are combined. Specifically, free base forms of drugs are reacted with carboxylic acids of differing chain length (eg acteric acid an butyric acid).

AD Complexes.

In devices comprising either simple or compound mobile phases, one or more essentially anhydrous Complexed Agent Depot Forms (CADFs) can be included. CADFs are capable of augmenting the delivery of therapeutic agent during the sustained release phase. CADFs are most often particulated solids, less than 50 microns in diameter, insoluble within the mobile phase, and release AD following exposure to the aqueous environment. In most embodiments most or all of the CADFs are protected from the aqueous environment at the implant site, by the presence of one or more mobile phase components, and/or one or more solid components. As the mobile phase elutes from the device the CADFs are exposed to the aqueous environment. The presence of a CADF often accelerates the adsorption of water, and in addition to their utility in providing an AD source to augment the sustained release phase. AD kinetics, they may also be used to accelerate device dispersion. Example 19 is a non-limiting example of CADFs and their preparation. Exemplary CADFs include carboxymethyl cellulose, carboxymethyl starch and hypromellose.

AD Saturated Mobile Phase.

In those cases where the mobile phase has been saturated with AD, additional AD may be added to the device in insoluble form. In this situation, additional AD may directly solubilize into the aqueous milieu after elution of the mobile phase. Example 4, Table 4.2 describes specific embodiments of this approach.

The approaches for decreasing the solubilization rate of a solid AD described above may also be combined with the approaches for the other two direct solubilization strategies described below.

### Control Point 1:Direct Solubilization of Reservoir Soluble AD.

For devices wherein the AD is soluble in a poorly water soluble or water insoluble reservoir, the AD will in some cases exit the reservoir by direct solublization (control point 1) based on its partitioning properties between the reservoir (or the entire mobile phase) and the aqueous milieu. Delivery kinetics through the partitioning route, may be prolonged by a.) controlling the concentration of the AD within the reservoir, b.) reducing the rate of elution of the reservoir from the device or c.) by altering the partitioning of the AD between the reservoir and the aqueous milieu.

Reservoir Concentration of the AD.

The duration of AD release from the reservoir is a function of its concentration, thus the higher the initial AD concentration within the reservoir the longer the duration of AD release. Mobile phases in general, and reservoirs in particular will often have significant hydrophobic character and desired ADs may have limited solubility within them. Strategies to increase AD solubility within the reservoir include: preparing hydrophobic salts of the AD free bases. Particularly useful for promoting hydrophobicity of free base drugs are organic acids, more specifically alkyl organic acids such as octanoic and lauric acid. Alternatively, limited amounts of tertiary substances capable of enhancing the AD solubility in the reservoir may be introduced, these include surfactants, ion pairing agents, or molecules intermediate in hydrophobic character between the AD and the reservoir (eg for a tocopherol reservoir, employ tocopherol polyethylene glycol succinate. See Table 4.5 & 26.2)

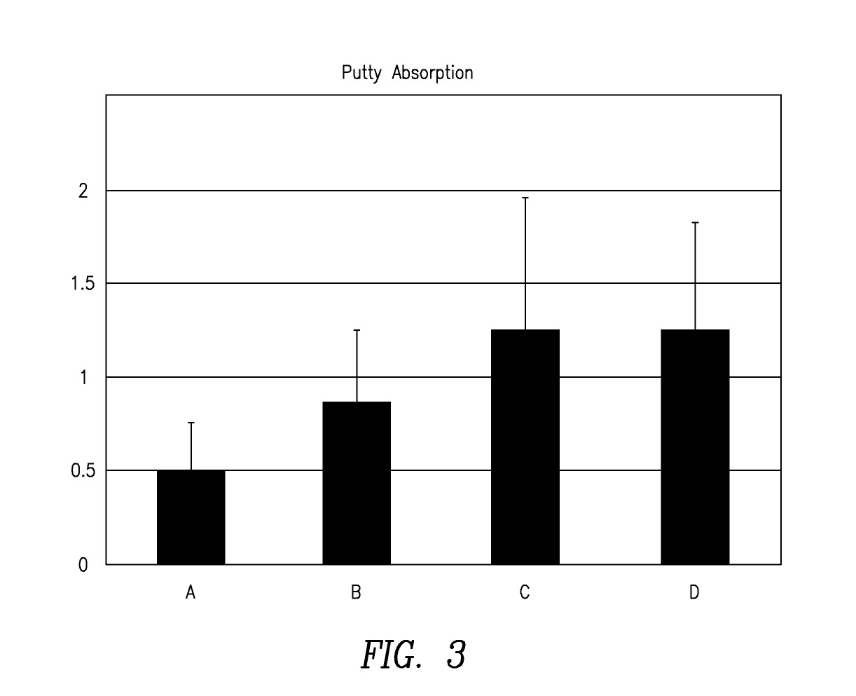

In an approach which combines control points 2 & 3, the AD is incorporated within in the device to an extent that more than saturates its concentration within the reservoir, thereby producing a pool of solid AD. As the AD departs the reservoir based on its partition coefficient between the reservoir and the aqueous milieu, the additional solid AD dissolves into the reservoir, in effect recharging it and prolonging the overall release of the drug Example 4, FIG. 3. When available, a reservoir may be selected into which the AD partitions to a greater extent (vs H20) than into the reference reservoir (ie tocopherol acetate). Examples of hydrophobic oils in which may be used as alternative reservoirs are provided in Example 4A & 4B.

### Control Point 1: Direct Solubilization of Second Solid Associated AD.

The AD may be entrapped by, embedded in, adsorbed to or encapsulated by a solid distinct from the AD in order to retard the kinetics of AD release

Entrapped or Embedded Complex.

A portion of the AD may be embedded within a second solid and incorporated within the inventive delivery formulations. In this embodiment, the entrapped AD is prevented or retarded from being released as compared to the AD in a non-entrapped formulation. In one embodiment, the release of the AD is retarded until some or all of the mobile phase elutes away from the device thereby exposing the entrapped complex to the aqueous environment and allowing release of the AD by direct solubilization (control point 1). In some embodiments the AD may diffuse out of a porous micronized solid depot such as a porous calcium phosphate (see Example 18). In other embodiments a micronized erodible plastic such as a polylactide, an absorbable polyurethane, a polyarylate, a polycaprolactone, or a tyrosine polycarbonate, is used and the embedded AD is released as the micronized solid dissolves or hydrolyzes upon exposure to aqueous body fluids. In preferred embodiments the AD is embedded within the micronized solid component of the device (see example 17) or within a solid mobile phase (see example 26.5)

Porous/Tortuous Path Elution:

In another embodiment, the AD is incorporated within a porous micronized substrate comprised of either erodible or permanent materials. By carefully controlling pore size and density of the solid substrate, the rate of elution of AD into the aqueous environment may be controlled. In some versions of this embodiment following release from the embedding solid the AD may be carried from the device by indirect solubilization through control point 2. In a preferred embodiment a micronized particulated fatty acid salt, or a micronized particulated cholesterol are prepared as porous materials with a pore size less than 50 microns and are used either as the micronized solid or a secondary solid in the inventive formulations. In one preferred embodiment, micronized fatty acid salts are melted in the presence of a softening agent (e.g. a liquid pluronic such as Pluronic L-35) and mixed with porogens or nano- or microparticulate ceramics, such as hydroxyapatite, bioglass or the like.

Calcium Phosphates and Calcium Sulfates.

AD may be incorporated within calcium phosphates by solid blending and pelletization, by co-precipitation with the solid calcium phosphate during its synthesis (Lee et al. U.S. Pat. No. 6,541,037) or by mixing with a settable calcium phosphate formulation such as described in Example 18 herein and/or as described by (Lee et al. U.S. Pat. No. 6,541,037) herein incorporated by reference in its entirety. The AD may also be embedded in other ceramics and or glasses including bioglasses, and phosphate glasses by methods known to those skilled in the art. In some instances the AD will be compressed with water soluble solids such as salts, polymers, or calcium compounds.

### Erodible Solid Forms.

AD may be embedded within water soluble, or hydrolytically or enzymically degradable solids. The solids containing embedded AD may be prepared in particle sizes less than 50 microns or may be particulated following preparation. The micronized solids thus prepared will erode and release AD in a controlled fashion following exposure to the aqueous environment. Methods to incorporate therapeutic agents into polymers and water erodible materials such as Poly Lactides, Tyrosine poly carbonates, Tyrosine polyarylates, Poly ethylene glycol, Solid surfactants (e.g. Poloxamers/Pluronics), Polycaprolactones, Polyorthoesters, and derivatives are known in the art and are described in The Handbook of Experimental Pharmacology 197 (2010): Drug Delivery, Monika Schäfer-Korting Springer-Verlag; Polymeric biomaterials (2002) By Severian Dumitriu—Marcel Dekker.—and the references contained therein

Examples 16 & 17 describe multiple phase delivery systems employing encapsulation in absorbable plastics as a means to retard AD delivery. (dual phase delivery—PLA encapsulated drug). Table 26.5 lists some delivery systems comprising water soluble Polaxomers and/or PEGs containing embedded AD.

Multiple AD Forms.

In another embodiment, the implant composition of the invention contains multiple forms of agent depots of a given agent in order to give a sustained delivery of that agent over time. The depots can be arranged homogeneously or at different depths within the implant so that as the implant gradually erodes, depots stored deeper within the implant disperse the agent at a higher rate. The correct arrangement of these depots allows for sustained release of the agent while the implant degrades. Exemplary embodiments include multiple alkyl organic AD salts of varying alkyl chain length. Another embodiment uses a variety of tocopherol salt derivatives of the AD such as tocopherol polyethylene glycol succinate, and tocopherol succinate.

### Control Point 2 AD is “Carried” by a Mobile Phase Component.

By definition, one or more of the mobile phase components of the invention are water soluble (ie soluble in the aqueous milieu). In addition for many embodiments of the invention, the AD is soluble in the mobile phase. Thus, in addition to the direct solublization of a solid or reservoir form of the AD as the underlying mechanism of delivery of AD, AD may also be delivered by co-solublization with one or more components of the mobile phase. The AD dissolves into the mobile phase which then exits the device carrying the dissolved AD with it. In this case (control point 2), AD kinetics may be modified by changing either its solubility within the mobile phase, or the elution kinetics of the mobile phase itself.

Adjustment of AD Solubility within the Mobile Phase

AD Salt Variants

When possible (eg drugs for which a free base form exist or which comprise a free carboxylic acid or Ammonium moiety) a variety of AD salts may be prepared to be either more or less hydrophobic to promote their solubility within the mobile phase. Alternatively detergents may be selected as components of the mobile phase for which the AD or one or more of its salts are more or less soluble according to need. Any appropriate methods known to the art to produce AD salts, for instance those described by Serajuddin (2007) or Giron (2003), may be used.

Adjustment of Mobile Phase Elution Rate.

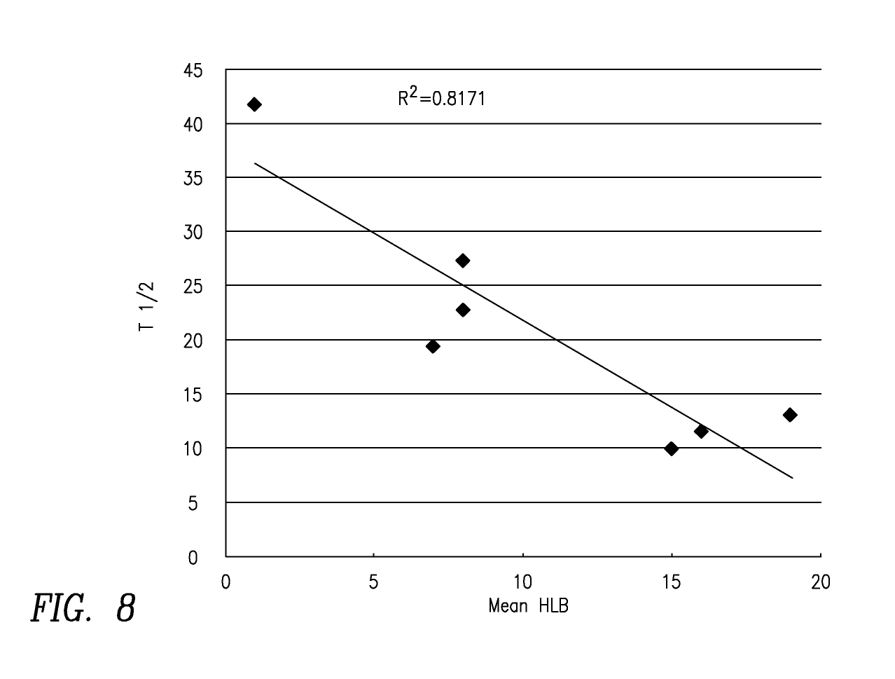

Selection of a mobile phase component having appropriate surface activity or detergency in which the AD is also soluble is also useful for controlling the elution rate of the therapeutic moiety. Specifically, Detergents with hydrophilic to lipophilic balance (HlB) values of less than 10 can reduce T1/2 of cumulative release of an AD by 50% as shown in FIG. 8. Carriers comprising a micronized solid, a reservoir (eg tocopherols acetate or SAIB) and a surfactant with an HLB(<19) and/or a CMC (<1) will be most useful for prolonging the sustained delivery of many ADs. Additional surfactants and their HLB values can be found in McCuthcheon's Emulsifiers and Detergents (2009).

Control Point 3 Interaction of the Mobile Phase with the AD Reservoir

Solid AD:

When the AD in solid form is prevented or retarded from exiting the device because of the presence of a reservoir (eg because it's coated with reservoir) and the AD delivery rate during the sustained release phase is below the rate required for therapeutic efficacy, the release rate of AD can be enhanced through promotion of a more rapid mobilization of the reservoir to thereby free up the AD to diffuse directly into the aqueous milieu. In these cases, a surfactant may be added to the reservoir to enhance the exit of either the reservoir or the AD from the device. For Poloxamer (Pluronic) soluble ADs, a Pluronic/reservoir combination such as shown for lidocaine in Table 4.4 is useful. Note in this system, the AD is soluble within both tocopherol and Pluronic to approximately the same amount (5-8% by weight). At 16% Lidocaine by weight, approximately half the lidocaine is present in dissolved form within the mobile phase and half is present as a solid. Thus the mobilization of the reservoir which occurs through the addition of increasing amounts of Pluronic ultimately mobilizes both soluble and solid AD.

Reservoir Soluble AD

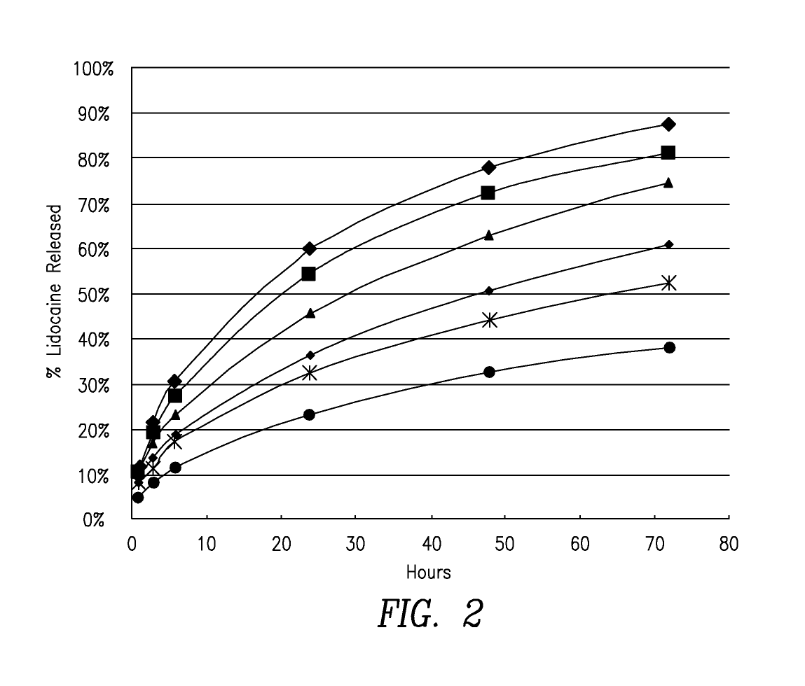

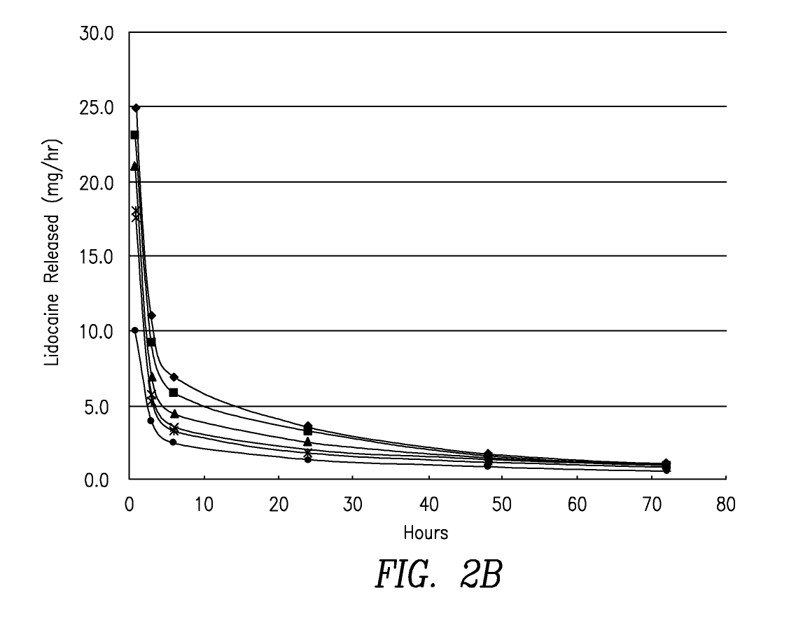

6% lidocaine is fully soluble within the reservoir of the formulation described in Table 4.5. The impact of increasing the concentration of surfactant is indicated by the release rate data in Table 4.5 and FIG. 2. In this instance the release rates of both the burst and sustained release phase are increased through the use of this form of kinetics modifier.

Reservoir Mobilization Using a Kinetics Modifier.

Surfactants are particularly useful for enhancing burst phase kinetics in such instances employment of surfactants with decreasing HLB values decreases burst see Example 20. Hydrophobic character resulting from this interaction affects mobilization of the reservoir and penetration of aqueous milieu necessary for drug delivery.

Examples of Mobile Phases Comprising a Reservoir with a Variety of ADs are as Follows:
a. Solubility of drug in reservoir
i. Example 4B (Vit K vs Vit E acetate)
ii. Example 12 (statins)
iii. Example 13 (paclitaxel)
iv. Example 14 (vaccine)
v. Example 23 (lyophilized protein)
vi. Example 24 (BMP-2)
   b. Salt vs free base form of drug affects dissolution of the drug into the reservoir
vii. Example 4C(HCl vs FB)
   Control Point 4 Interaction of the AD with the Micronized Solid.

Interaction of the AD with either the micronized solid or a second included solid may be exploited to extend AD retention time (prolong delivery) within the device. In general solids having specific chemical identities will be chosen which, in addition to meeting the requirements of insolubility within the mobile phase, are expected to have significant interactions with the AD, such that the interaction with the solid will retard their migration from the device. Typical bases of interaction include the hydrophobic interaction, ionic interaction, hydrogen bonding and AD diffusion within pores of the micronized solid. Solids with greater ability to interact with the soluble AD will prolong elution to the greatest extent. Most often, techniques exploiting control point 4 will be utilized to retard the elution of dissolved AD (dissolved within the reservoir or mobile phase).

To further promote interaction of the AD with the solid phase, the AD may be modified (eg varying the salt form). Specific details regarding prolonging elution by exploiting each of the various interactions are presented below.

Hydrophobic Interaction

Most often the hydrophobic interaction with the AD and the micronized solid is promoted by preparing extremely hydrophobic salts of the AD such as fatty acid, cholesterol, or tocopherols salts. Examples of compounds that may adsorb ADs by hydrophobic interaction/partitioning with liquid or otherwise solubilized ADs are C18 reverse phase chromatography substrates and resorbable equivalents, including ceramics. Delivery of AD using preferred micronized hydrophobic solids is described in Example 4E (varying metal stearate salts). Alternatively, as described in the next section, solubilization of ADs in surface active liquids (eg surfactants) known to interact with the micronized solid may be employed to prolong delivery times

Preferred Second Solids

Any solid capable of interacting with the AD and retarding its elution from the device as in the case of a ceramic such as calcium phosphate, may be employed as a second solid within the device. Second solids may be incorporated into the device by as much as 60% by weight with particle sizes ranging from nanometer to not more the about 500 microns. Preferred second solids will have mean particle sizes of between 100-300 microns. Suitable second solid interactions with the AD are the same as those described for AD/micronized solid interactions including tortuous path (diffusion into a porous second solid) ionic interaction, hydrophobic interaction, and hydrogen bonding. Preferred second solids include ceramics, bioglasses, magnesium and strontium phosphates and substituted calcium phosphates (eg silicate, strontium, magnesium, aluminum, or manganese substituted) and calcium phosphates such as alpha and beta TCPs, hydroxyapatites, poorly crystalline hydroxyapatites and any resorbable amorphous ceramics including amorphous hydroxyapatite. Other suitable second solids include absorbable polymers, and copolymers, polysaccharides, glycosaminoglycans etc.

Preferred AD Salts

Ionic Interaction

ADs in some cases may be adsorbed to charged micronized particles or other forms compatible with and insoluble in, the mobile phase (eg. second solids). In general for all these charged materials, an anhydrous preparation of the AD and the adsorbing material will be prepared under conditions which promote the adsorption, binding or interaction of the AD with the absorbing substrate. The complex will either be prepared in such a way that the particles are fifty microns or less, or the complex will be subjected to particle size reduction (e.g. grinding, milling, etc) prior to preparing the device. The AD so adsorbed will either be used as the sole solid form or will be mixed with a carrier such as calcium stearate to produce the desired handling properties.

Whether present as the micronized carrier or a second solid, porous ceramics such as particulate calcium phosphates are useful to prolong the duration of delivery of proteins and nucleic acids (example 23).

The following compounds are examples of compounds that adsorb ADs by ionic or hydrogen bonding: fatty acids, polar phospholipids, polysaccharides, hyaluronic acid, starch, chitosan, alginate, agar, protein/peptides, nucleic acids, collagen, albumin, carboxymethyl cellulose and oxidized cellulose, calcium phosphates, calcium sulphates, ethylene diamine tetra-acetic acid, Diethylaminoethyl cellulose and absorbable analogues and derivatives thereof.

Covalent Complex

AD delivery kinetics from the inventive devices may also be altered by covalent reaction with moieties on the micronized carrier or on the second solid. This approach is particularly useful for the delivery of nucleic acids and/or proteins or molecules containing poly saccharides. In these cases the AD is linked to the device by a hydrolysable or enzymatically cleavable linker, such as a poly lysine or poly arginine peptide. Following implantation, circulating enzymes (eg proteases, nucleases, hydrolases, glycosylases and glycosidases) cleave the AD from the linker allowing it to elute from the device.

### Control Point 5 Interaction Between One or More Components of the Mobile Phase and the Micronized Solid:

The ability of the micronized solid phase to affect the disappearance of the mobile phase, and the tuning thereof, is one of the mechanisms by which the invention affects the overall capacity of the device with respect to the therapeutic agent as well as the rate of delivery of the therapeutic agent. The micronized solid phase is generally a.) insoluble or at least poorly soluble in water, b.) less polar than water (or has at least a partial hydrophobic character) and in some cases is c.) capable of an hydrophobic or affinity interaction with an appropriate second molecule (e.g. AD, reservoir, or mobile phase component).

Following implantation into the aqeous milieu of the body, part or all of the mobile phase is retained as a part of the carrier for an extended period of time due to either or both of a.) its solubility limitations within the extracellular fluid environment, b.) its affinity for or partitioning into the solid phase (e.g. through hydrophobic interaction, Van der Waals forces or other affinity or adsorption means). Mechanism notwithstanding, the mobile phase is retained at the implant site for a period of time appropriate to the desired rate of release of the Agent Depot, and longer than the retention of the mobile phase would occur if it were either free in solution or in combination with a non- or less-hydrophobic substrate. High viscosity mobile phase components, also may be employed to prolong device coherence in vivo.

Exemplary compound mobile phases and matched micronized solid phases which are expected to interact are presented throughout the Examples. These pairs comprise both kinetics modifiers and reservoir components which are capable of interacting with the corresponding micronized solid. Preferred basis of interaction between the mobile phase and a corresponding matched micronized solids include: Hydrophobic interaction, Van der Waals forces, chelation through a chelating surfactant, ionic bonding, hydrogen bonding

Mobile phase elution can be retarded by establishing interactions with the micronized solid component. Any or all of the approaches described above for control point 4 may be exploited to enhance retardation of the mobile phase from reference formulations similar to and including the base carrier (Table 2.1, Table 1.2) by altering hydrophobic interaction tortuosity, porosity, packing density, or particle size of the micronized solid.

Sorbitan oleate was chosen as a potential kinetics modifier because of the alkyl chain it contains and the likelihood that it would interact with the stearate moeity of the micronized solid.
Control Point 6 Ingress of Water into the Device.

Materials may be incorporated into the device which promote ingress of water into the device. In addition to the role of these compounds in accelerating absorption of the device, they can also be used to induce elution from devices exhibiting suboptimal AD release. Generally, these materials will be water soluble, or water binding. In preferred embodiments they will stay associated with the device for several days or longer. They are particularly useful for reservoirs in which the AD is soluble, but which release AD at suboptimal rates. Lyophilized hydrogels often serve as suitable water imbibing vehicles. Other water imbibing additives include: poorly hydrated or substantially dehydrated forms of tissue fragments, demineralized bone fragments proteins and polysaccharides, proteoglycans, glycosamino glycans, collagen, gelatin, alginate, hyaluronic acid, high molecular weight polyethylene glycols, chitosans, and synthetic polymers.

Example 19 demonstrates the use of a hydrogel to promote elution of an anesthetic free base from a tocopherol reservoir. Preferred water imbibing substances include carboxymethyl cellulose carboxymethyl starch and hypromellose and their derivatives.

### Strategies Involving Multiple Control Points:

Other strategies for the control of AD release from the inventive devices exist which do nitas clearly fit into just one of the six control points described above. Some of these include:
The delivery of drugs, poorly soluble drugs or water insoluble drugs dissolved in wax-like reservoirs as described in Example 15 is similar in approach to the delivery of drugs from solid dispersion formulations known in the oral drug fabrication industry and as described by Vasanthavada et al. 2008 in Water-Insoluble Drug Formulation 2^{nd} edition (Edited by Rong Liu CRC Press pg 499-530) herein incorporated by reference. In these formulations the drug is dispersed in a water soluble waxy mobile phase. As the mobile phase elutes it may un mask some or all of an insoluble AD, and/or complex with or dissolve the AD and transport it away from the device.
Replacement of the all or some of the micronized solid with a soluble solid mobile phase. As described in Example 8 (PEG/Pluronic)
Solubilization-dependent pH modification: Some or all of the micronized solid component maybe fabricated from an hydrolysable component capable of releasing acidic or basic hydrolysis fragments in the presence of water at the implant site (e.g. polylactide, polyglycolide, polyamines, poly aminoacids). The local pH change due to the presence of these hydrolysis fragments results in the release of AD ionically bound to a second or the same micronized component. Achievement of a specific pH may be engineered to create a time dependent secondary burst phase of AD.

### Tuning Therapeutic Agent Capacity

In other embodiments, the compositions of the invention are tuned to effect the device capacity for the AD. This can be done by using blends of solid carrier. For example, calcium stearate can be blended with other solid carriers to change the therapeutic agent capacity. These additional solid carriers include hydrogels like poly (D,L) lactide (PLA). Further, anesthetic liquids can be used that are also used as mobile phases of the implants of the invention. Further, agents made up of erodible solids can themselves be used as solid carriers or may be blended with other solid carriers to modulate agent capacity.

Device Absorption

The rate of absorption of the inventive devices can often times be altered independently of the drug elution rate. In preferred embodiments, absorption is accelerated by reducing the hydrophobic character of the micronized salt (eg Example 5; replacement of calcium stearate with palmitate, laurate, or oleate salts). Specifically, for carriers comprising alkyl salts (e.g. fatty acid salts) the chain length may be reduced. Other strategies include the replacement of some or all of the micronized carrier with one or more of an erodible second solid (e.g. example 6; use of 2nd solid—erodibles), water soluble dispersants (e.g. example 7; use of 2^{nd} solid−dispersants), a water soluble or otherwise hydrophilic carrier such as a PEG or Solid Pluronic (e.g. example 8; PEG/Pluronic+hydrophilic primary solid), a substantially dehydrated hydrogels (e.g. example 19), or increasing the surface area of the micronized solid (reduce the average particle size of some or all of the micronized solid).

Structural Enforcement

AD delivery from many of the inventive devices is diffusion driven, with specific kinetic phases that can be defined by Fick's law. For many of the inventive devices, the thickness of implanted device controls the ultimate delivery characteristics. Because many of the inventive devices are malleable putties, it may be advantageous in many cases to control the thickness that the putty may be applied. This may be accomplished by the incorporation of controlled size spacers—beads, particles—rods, strips, etc. in to the device in order to maintain a fixed minimal thickness of the putty device.

Note the use of structural elements within the device allows an alternative entrapment strategy to prolong or accelerate release of the AD during the sustained release phase, since the AD may also or alternatively be embedded within the structural elements to elute as described above.

### Accessories/Fillers, Add-Ins, and Combinations of Therapeutic Agents

The compositions of the invention may also include other accessories, fillers, add-ins and combinations of agents including mesh, whiskers, fillers, sutures, suture anchors, multiple therapeutic agents and clotting agents.

According to specific embodiments of the invention, an absorbable mesh is used to coat the delivery devices of the invention. This mesh comprises a dehydrated hydrogel and optionally, induces blood clotting. In another specific embodiment, the mesh is used to produce a gauze. This gauze comprises a dehydrated hydrogel and optionally, induces blood clotting. This gauze can be arranged to surround a delivery device according to the invention or can be embedded in such devices.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

Clinical Use and Surgical Implantation.

The compositions of the invention are administered through surgical techniques known in the art. Surgical implantation of the drug delivery system could include a) hand molding of the drug delivery system to the configuration appropriate for the indication, b) injecting or extruding the drug delivery system through a standard or modified syringe, or c) applying the drug delivery system using an applicator. Anesthetic applications included implantation of a local anesthetic releasing device in the vicinity of a nerve to create regional nerve block. In one specific delivery embodiment, a gauze mesh or other thin strip is prepared such that the inventive putties can be loaded or spread on one side. The mesh thus prepared is introduced into the body putty side down towards the tissue. An pressed into place, as either a hemostat, or drug delivery vehicle. The strip may be left implanted with the putty or alternatively the mesh is removed through the use of tab or string incorporated into the mesh, leaving the putty behind. Preferred mesh materials include resorbable plastics, oxided cellulose, surgical metals such as stainless steel or titanium, cotton, cotton gauze and the like.

### Protein Delivery Applications

Also within the inventive concept is an implantable, moldable putty that provides one or more BMPs, osteogenic, osteopromotive or osteoinductive or osteoconductive proteins to promote bone healing and/or growth. The putty is anhydrous and contains osteoconductive “delivery particles” to which BMP is adsorbed. The BMP is adsorbed onto the osteoconductive “delivery particles” and dried. Particle size ranges from about 25 to about 500 microns. Dried loaded delivery particles are mixed with a micronized solid (e.g. calcium laurate), a liquid poloxamer (e.g. Pluronic L-35) and optionally a tocopherol to produce a formable putty. The putty is implanted into a site within the body where improved bone healing is required. In a variation un-adsorbed lyophilized BMP is further blended into the putty.

One embodiment is directed to an implantable and moldable composition for the local delivery of a protein to a subject, comprising: a substantially water free matrix and a mobile phase comprising the protein. In another embodiment, the protein is a bone morphogenetic protein. In another embodiment, the matrix comprises an osteoconductive ceramic or calcium compound. In another embodiment, the composition further comprises calcium laurate, Pluronic L-35 and/or tocopherol. In another embodiment, a lyophilized bone morphogenetic protein is blended directly into the composition without being adsorbed.

Another embodiment is directed to a method for treating a bone disorder or injury, comprising delivering a bone morphogenetic protein to a subject at the site of the disorder or injury comprising administering a composition described herein at the site of the disorder or injury. The composition, e.g., putty, is implanted into a site within the body where improved bone healing is required.

Matrix

In specific embodiments of the protein delivery application, the formulation is substantially water free (anhydrous) comprising micronized particles (<100 20 microns, preferably about 50 microns or less) of a divalent fatty acid salt, e.g., calcium laurate or calcium stearate.

Insoluble in the Mobile Phase

In specific embodiments of the protein delivery application, the micronized particles are suspended in an organic mobile phase (either liquid or solid). The formulation is absorbable/resorbable by the body in vivo, following or as part of, delivery of the agent.

Mobile Phase

In specific embodiments of the protein delivery application, an organic liquid or solid characterized by its time-dependent solubilization following implantation in the body, e.g., Pluronic/tocopherol blends, polyethylene glycol and blends thereof, SAIB and blends thereof and triethyl citrate. By itself the mobile phase solubilizes neither the micronized particles nor the delivery particles.

Delivery Particle

In specific embodiments of the protein delivery application, the formulation includes delivery particles which are insoluble within the matrix mobile phase in the absence of water. Suitable sizes of the delivery particle range from about 1 to about 5000 microns, about 10 to about 1000 and about 50 to about 300 microns. Most often the delivery particles are osteoconductive and are formed from materials such as calcium phosphates, bioglasses, osteoconductive polymers. When implantation in or near bone is not required, non-osteoconductive delivery particles may be used.

Delivery of Agent

In specific embodiments of the protein delivery application, the agent to be delivered is associated with the delivery particle can be chosen according to requirements of the implant site and clinical need. For example, for bone healing, delivery particles can be prepared from osteoconductive materials including osteoconductive ceramics, polymers or glasses either in particulate or whisker (fibrous) form. Osteoconductive materials include, but are not limited to, calcium phosphates, calcium sulfates, bioglasses, bone, bone derivatives including demineralized bone, collagen-derived or collagen-containing particles, cancellous or cortical bone particles chips or fragments, tyrosine polycarbonates and tyrosine polyarylates.

Delivery particles for agents for non-bone healing applications can be composed of, for example, any of the above and oxidized cellulose, insoluble tissue preparations and absorbable polymers, hydrogels such as, for example, agar, alginate and chitosan. For delivery of charged agents, a particle capable of ion exchange can be used, e.g., oxidized cellulose, DEAE-derivatized particles, carboxymethylated particles, etc. For all of the above, some or all of the agent can be incorporated inside the particles. In such instances, a water-hydrolyzable or enzymatic-degradable particle can be used.

Molecules for Delivery

Proteins: most proteins can be absorbed directly form aqueous solutions onto, for example, calcium phosphate particles. BMPs, e.g., BMP-4, BMP-7 and other bone growth promoting proteins, insulin, insulin-like growth factor, TGF-beta, PDGF, osteocalcin, neuropeptides, substance P, and CGRP are useful for bone growth. Other molecules that can be delivered include, but are not limited to, for example, nucleic acids, organic molecules, glycoproteins, proteoglycans etc.

### EXAMPLES

### Example 1

### Preparation of Drug Releasing Putties

In the examples that follow, the drug delivery formulations presented (consisting of up to two solids and three liquids; e.g. kinetics modifier+reservoir+drug), may be expressed either 1.) as the weight % of the individual components, or 2.) in terms of a drug-free carrier composition to be mixed with a drug. Specifically, in many of the formulations presented herein, the drug content is at 16% by weight. The expression of these compositions in drug-free carrier terms considers them as a formulation of components to be mixed with a drug at a ratio of 84:16 (wt/wt). Once the weight % of all components is known, the weight % of the drug-free carrier components is calculated as follows:

$$Solid 1 A = ( Solid 1 + Solid 2 + \frac{drug}{2} ) * ( \frac{Solid 1}{Solid 1 + Solid 2} )$$

$$Solid 2 A = ( Solid 1 + Solid 2 + \frac{drug}{2} ) * ( \frac{Solid 2}{Solid 1 + Solid 2} )$$

$$Liquid 1 A = ( Liquid 1 + Liquid 2 + Liquid 3 + \frac{drug}{2} ) * ( \frac{Liquid 1}{Liquid 1 + Liquid 2 + Liquid 3} )$$

$$Liquid 2 A = ( Liquid 1 + Liquid 2 + Liquid 3 + \frac{drug}{2} ) * ( \frac{Liquid 2}{Liquid 1 + Liquid 2 + Liquid 3} )$$

$$Liquid 3 A = ( Liquid 1 + Liquid 2 + Liquid 3 + \frac{drug}{2} ) * ( \frac{Liquid 3}{Liquid 1 + Liquid 2 + Liquid 3} )$$

Once the weight percents of the components of the drug-free carrier compositions have been determined, a drug may be mixed into the carrier according to the doseage required. Comparisons of the two ways of considering the same composition containing 16% lidocaine are shown in Table 1.1.

TABLE 1.1
A formulation expressed as wt % of all its components or as
a drug-free composition to be mixed 84:16 (wt %) of drug.

| | | | | | | | 16% | |
|---|---|---|---|---|---|---|---|---|
| | | 84% Carrier | | | | | Drug | |
| | | TCP | CS | P123 | F68 | TA | LFB | |
| line | Drug Formulation | solid1 | solid2 | liquid1 | liquid2 | Liquid 3 | drug | Total |
| 1 | Component weight % | 40 | 15 | 16 | 8 | 5 | 16 | 100 |
| 2 | Carrier Composition which | 46 | 17 | 20 | 10 | 6 | — | 100 |
| | when mixed 84:16 with drug | | | | | | | |
| | yields composition of line 1 | | | | | | | |

Abbreviations: TCP—tricalcium phosphate; CS—calcium stearate; P123—Pluronic P123; F68—Pluronic F68; TA—tocopherol acetate; LFB—lidocaine free base

Expression of the formulation as given in line 2 allows for the simple inclusion of any % drug into a constant carrier formulation simply by varying the relative ratio of carrier to drug.

### Anesthetic Putties.

A variety of anesthetic delivery formulations were prepared and in vitro release kinetics were assessed. Results are presented in Table 1.2.

TABLE 1.2
Formulations of Exemplary Anesthetic Putties

| | Cumulative | Release Rate (mg/mL) | | | | Time |
|---|---|---|---|---|---|---|
| | Release at | 1 | 24 | 48 | 72 | to 1.8 |
| Putty Formulation* | 72 hrs. (%) | hr | hr | hr | hr | mg/hr |

Lidocaine Free Base Delivery Formulation

| | | | | | | |
|---|---|---|---|---|---|---|
| 55% CS/24% L-35/ | 84% | 22.5 | 3.3 | 1.7 | 0.8 | 46 |
| 5% TA/16% LFB | | | | | | |

Lidocaine HCl Delivery Formulations

| | | | | | | |
|---|---|---|---|---|---|---|
| 55% CS/24% L-35/ | 74% | 55.3 | 1.4 | 0 | 0 | 22 |
| 5% TA/16% LHCl | | | | | | |
| 55% CS/24% L-35/ | 81% | 36.4 | 2.6 | 0.8 | 0.5 | 30 |
| 5% TA/8% LFB/8% HCl | | | | | | |

Bupivacaine Free Base Delivery Formulations

| | | | | | | |
|---|---|---|---|---|---|---|
| 60% CS/27% L-35/ | 48% | 2.8 | 1.0 | 0.5 | 0.3 | 6 |
| 5% TA/8% BFB | | | | | | |
| 60% CS/24.5% L-35/ | 41% | 2.4 | 0.8 | 0.4 | 0.3 | 2.75 |
| 7.5% TA/8% BFB | | | | | | |
| 60% CS/22% L-35/ | 39% | 2.3 | 0.8 | 0.4 | 0.3 | 2 |
| 10% TA/8% BFB | | | | | | |
| 60% CS/17% L-35/ | 30% | 2.3 | 0.5 | 0.4 | 0.3 | 1.25 |
| 15% TA/8% BFB | | | | | | |

Tetracaine Free Base Delivery Formulation

| | | | | | | |
|---|---|---|---|---|---|---|
| 55% CS/24% L-35/ | 58% | 14.6 | 2.5 | 1.1 | 0.6 | 36 |
| 5% TA/16% TFB | | | | | | |

CS—calcium stearate; L-35—Pluronic L-3; TA—Tocopherol acetate; LFB—lidocaine free base; LHCL—lidocaine HCl; BFB—Bupivicaine free base; TFB tetracaine free base

### Processing: Solution Production and Filtration

Formulations presented in the examples were prepared by the following general procedure: A clean 100 or 150 mL beaker (depending on size of putty needed) was placed on a balance and tared. The calculated amount of the drug to be delivered (e.g. Lidocaine Free Base, other anesthetic agents or other drugs) was weighed into the beaker and then the balance was tared. The calculated amount of tocopheryl acetate was placed into the same beaker via transfer pipet and the balance tared. This same step was repeated once more for the liquid components using a clean transfer pipet. The beaker was placed on a warmed hot plate. Once the components were melted and homogenous, the beaker was returned to a freshly tared balance and the micronized particulate (e.g. calcium stearate) and other solids if any, were added and the components slowly mixed with a stainless steel spatula.

Once the particles began to aggregate into small clumps, and unaggreagated material minimally visible, the product was hand mixed and kneaded. Any free powders were incorporated into the batch by dabbing the putty against the beaker to adhere to it. Once the mixture reached a doughy consistency, the end product was hand mixed/kneaded for approximately an additional 1-2 minutes to ensure complete homogeneity of the components.

### Elution Testing and Analysis

The following is a description of the “Test Elution Assay”: Putty samples were formed into disks in a washer shaped mold (diameter—21 mm, thickness—4 mm), placed in a nylon biopsy bag, closed using a dialysis closure clip and placed in a VK 7000 dissolution bath containing 900 ml of 50 mM potassium phosphate buffered solution (pH=7.4) pre-warmed to 37° C. and set to a paddle speed of 25 RPM. At predetermined time intervals, 5 ml of the elution bath solution was collected for subsequent lidocaine free base detection and replaced with 5 ml of fresh solution.

Detection of lidocaine free base was assayed either by measuring absorbance at 234 nm, using a UV/VIS Lambda 2 spectrophotometer calibrated with standard solutions of known lidocaine concentrations or by reverse phase analysis on a Phenomenex Gemini-NX 3u C18 110A column (50×3.0 mm) with a Perkin Elmer Series 200 HPLC
Mobile Phase A: Methanol
Mobile Phase B: 10 mM Ammonium Bicarbonate in Water
Temperature: 30° C.
Run Time: 15 minutes
Injection Volume: 20 uL
Detection: absorbance of 254 nm

TABLE 1.3
HPLC Gradient Elution Parameters

| | Time | Flow | Mobile | Mobile | |
|---|---|---|---|---|---|
| Step | (min) | (mL/min) | Phase A | Phase B | Curve |
| 0 | 0.5 | 0.5 | 60 | 40 | 0.0 |
| 1 | 5.0 | 0.5 | 60 | 40 | 0.0 |
| 2 | 1.0 | 0.5 | 95 | 5 | 1.0 |
| 3 | 0.5 | 0.5 | 95 | 5 | 0.0 |
| 4 | 0.5 | 0.5 | 60 | 40 | 1.0 |
| 5 | 8.0 | 0.5 | 60 | 40 | 0.0 |

### Example 2

### Quantitative Assessment of Handling and Formulation Consistency

The handling properties of the base carrier and the base carrier plus 16% Lidocaine (Orthostat and Orthostat-L respectively) were evaluated as follows:

### Test Article Preparation—Orthostat

The base carrier composition, Orthostat, and four ‘wet’ formulations and four ‘dry’ formulations were fabricated and placed in high-density polyethylene (HDPE) jars with low-density polyethylene (LDPE) lids (Tables 2.1, 2.2, and 2.3).

TABLE 2.1
Base Carrier Composition

| (% w/w) | Orthostat |
|---|---|
| Calcium Stearate | 60 |
| Pluronic L-35 | 35 |
| dl-α-Tocopherol Acetate | 5 |

TABLE 2.2

| (% w/w) | Wet 1 | Wet 2 | Wet 3 | Wet 4 |
|---|---|---|---|---|
| Calcium Stearate | 57.5 | 55 | 52.5 | 50 |
| Pluronic L-35 | 40 | 40 | 42.5 | 42.5 |
| dl-α-Tocopherol Acetate | 2.5 | 5 | 5 | 7.5 |

TABLE 2.3

| (% w/w) | Dry 1 | Dry 2 | Dry 3 | Dry 4 |
|---|---|---|---|---|
| Calcium Stearate | 62.5 | 65 | 67.5 | 70 |
| Pluronic L-35 | 32.5 | 30 | 30 | 27.5 |
| dl-α-Tocopherol Acetate | 5 | 5 | 2.5 | 2.5 |

### Experimental Design—Base Carrier

Putty formulations were subjected to a series of qualitative and quantitative tests to analyze their handling characteristics. The qualitative test criteria included an analysis of: Color, Stickiness, Coherence, and Molding ability. The quantitative test criteria included Penetrometer analysis

### Procedure—Base Carrier

The color of the samples was examined and scored as follows:
Score 3—off-white putty
Score 2—medium
Score 1—dark

The putty was split into three pieces for each of the following applications: Sample 1 was used for qualitative assessment of stickiness. of the sample was divided into sub samples of 3-5 grams were rolled into a ball in the palm of the hand. Gloves were examined for residue and scored on the following 3 point scale.
Score 3—putty did not stick to gloves
Score 2—some putty stuck to gloves
Score 1—putty severely stuck to gloves

Sample 2 was used for qualitative assessment of coherence by rolling the sample into a ball and applying pressure to the center of the ball with the thumb. The circumferential area of the putty was examined for the degree of cracking or crumbling and was scored on the following three point scale:
Score 3—no cracking occurred
Score 2—some cracking occurred
Score 1—cracking occurred

Sample 3 was evaluated for molding ability by squeezing and releasing the putty in the palm of the hands. The gloves were examined for residue and the putty was examined to determine whether the putty held its shape and scored on the following three point scale:
Score 3—putty did not stick to gloves and held its structure
Score 2—putty stuck to gloves and somewhat held its structure
Score 1—putty stuck to gloves and did not hold its structure well

The firmness of sample 4 was assessed quantitatively with a penetrometer. A new piece of untouched sample held at room temperature for at least 4 hours was used. First the penetrometer (Koehler) was calibrated. Then the sample was packed into a cylindrical plastic vial (diameter ¾ in, height ¼ in), leveled and smoothed. The sample was placed on a platform at the base of the penetrometer. A 50 g weight was placed on top of the plunger rod and the head of the penetrometer was lowered so that the tip of the cone was as close as possible to, without touching, the surface of the sample. Each sample was tested in triplicate.
Score 3—penetrometer value was 45±5
Score 2—penetrometer value was 45±10
Score 1—penetrometer value was 45±15

### Results—Base Carrier

The results are summarized in the tables below:

TABLE 2.4
Base Carrier - nominal

| | Normal |
|---|---|
| Color | 3 |
| Stickiness | 3 |
| Coherence | 3 |
| Molding ability | 3 |
| Penetrometer | 3 |
| Total | 15 |

TABLE 2.5
Base Carrier Prepared Wet

| | Wet 1 | Wet 2 | Wet 3 | Wet 4 |
|---|---|---|---|---|
| Color | 3 | 3 | 3 | 3 |
| Stickiness | 3 | 3 | 2 | 1 |
| Coherence | 3 | 3 | 3 | 3 |
| Molding Ability | 3 | 3 | 3 | 2 |
| Penetrometer | 3 | 2 | 1 | 1 |
| Total | 15 | 14 | 12 | 10 |

TABLE 2.6
Base Carrier Prepared Dry

| | Dry 1 | Dry 2 | Dry 3 | Dry 4 |
|---|---|---|---|---|
| Color | 3 | 3 | 3 | 3 |
| Stickiness | 3 | 3 | 3 | 3 |
| Coherence | 3 | 2 | 2 | 1 |
| Molding Ability | 3 | 3 | 3 | 2 |
| Penetrometer | 1 | 1 | 1 | 1 |
| Total | 13 | 12 | 12 | 10 |

The means of 3 replicates were compared with the results of the standard putty formulation. In the samples classified as ‘Wet’, the Stickiness and the Penetrometer tests demonstrated the greatest differences compared to the standard Orthostat formulation. In the samples classified as ‘Dry’, the tests demonstrating the greatest differences were the Coherence and Penetrometer tests.

Testing of the standard formulation yielded a total score of 15 out of a maximum score of 15. The formulations classified as ‘Wet’ and ‘Dry’ yielded total scores ranging from 10 to 15 with the majority of scores falling in the 10-12 range.

These results demonstrate that the scoring system is effective in distinguishing between formulations with good and bad handling characteristics. The minimum score required to Pass was set at 14 which allows for an imperfect score (2) on a maximum of 1 test. A score of less than 14 results in a fail.

### Test Article Preparation—Orthostat-L

Orthostat-L which contains the base carrier formation plus 16% Lidocaine Free Base and was fabricated along with ‘wet,’ ‘very wet,’ ‘dry,’ and ‘very dry’ formulations. The formulations were stored in high-density polyethylene (HDPE) jars with low-density polyethylene (LDPE) lids until testing. The following test articles were used for these analyses:

TABLE 2.7

| (% w/w) | Orthostat-L | Very Wet | Wet | Very Dry | Dry |
|---|---|---|---|---|---|
| Calcium Stearate | 55 | 50 | 52.5 | 60 | 57.5 |
| Pluronic L-35 | 24 | 27 | 25 | 20 | 22 |
| dl-α-Tocopherol | 5 | 7 | 6.5 | 4 | 4.5 |
| Acetate | | | | | |
| Lidocaine Free Base | 16 | 16 | 16 | 16 | 16 |

### Experimental Design—Orthostat-L

The putty formulations (Table 2.7) were subjected to the same set of tests described above (Example 2).

### Procedure—Orthostat-L

The test procedure used for assessment of putty formulations in Table 2.7 are the same as those described above (Example 2).

### Results—Orthostat-L

The results are summarized in the tables below:

TABLE 2.8

| | Orthostat-L | Very Wet | Wet | Very Dry | Dry |
|---|---|---|---|---|---|
| Color | 3 | 3 | 3 | 3 | 3 |
| Stickiness | 3 | 1 | 2 | 3 | 3 |
| Coherence | 3 | 3 | 3 | 1 | 1 |
| Molding Ability | 3 | 2 | 3 | 1 | 2 |
| Penetrometer | 3 | 1 | 2 | 1 | 1 |
| Total | 15 | 10 | 13 | 9 | 10 |

The means of 3 replicates were compared with the results of the standard putty formulation. In the samples classified as ‘very wet’ and ‘wet’, the stickiness and the penetrometer tests demonstrated the greatest differences compared to the standard Orthostat-L formulation. In the samples classified as ‘Very Dry’ and ‘Dry’, the tests demonstrating the greatest differences were the coherence, molding ability and penetrometer tests.

Testing of the standard formulation yielded a total score of 15 out of a maximum score of 15. The ‘wet’ and ‘dry’ variations of the standard formulation yielded total scores ranging from 9 to 13.

These results demonstrate that the scoring system is effective in distinguishing between formulations with good and bad handling characteristics. The minimum score required to Pass was set at 14 which allows for an imperfect score (2) on a maximum of 1 test. A score of less than 14 results in a fail.

### Example 3

### Assessment of Water Resistance

This example describes the procedure to assess water resistance of the inventive formulations. Approximately 1 gram of putty, as described in example 2, table 2.1, was weighed and placed into a 500 mL beaker filled with 250 mL of phosphate buffer. Using gloved hands, the sample was held under the surface of the buffer and formed into a six sided cube-like shape, and subsequently flattened into a disk. After the submerged sample had been manipulated for fifteen seconds, the disk of putty was pressed against the wall of the beaker while still submerged until it adhered to the beaker wall. The sample was then immediately retrieved from the beaker by scooping it up the side of the beaker wall with two fingers in a single swipe. The putty was then formed into a ball with fingertips, dried on a Kimwipe® for 5 seconds and weighed a second time.

The percent of the starting mass lost in the beaker as a result of the manipulations described above was calculated according to the following formula:

(Initial Weight−Final Weight)*100

### Initial Weight

If the mass loss was less than 10%, the sample was considered highly cohesive and water resistant. Mass loss between 10 and 50% indicates moderate cohesiveness and water resistance. Less than a 50% recovery is indicative of marginal cohesiveness and water resistance.

### Example 4

### Reservoirs and Modification of Drug Release Kinetics

### Exemplary Reservoirs

This example demonstrates how various liquids/solids/semi-solids can be used as reservoirs as part of the technology described in this application. Table 4.1 (reservoirs are bolded) gives examples of multiple liquids/solids/semi-solids and their effect on sequestering drug into the reservoir phase. Examples of reservoirs include dl α-tocopherol acetate (TA), tocopheryl succinate (TS), Tocopherol Polyethylene Glycol 1000 Succinate (TPGS), Sorbitan Monooleate, SAIB, Pluronic 121(L121) and Vitamin K (VK). In this example, the weight percentage of calcium stearate was kept between 55% and 60%, and lidocaine free base was kept between 15% and 16%. The weight percentage of the reservoir was varied between 5% and 29%. Depending on the affinity of the reservoir for the drug and the amount of the reservoir added, large differences in the % drug released at 72 hours were achieved.

TABLE 4.1

| | % released | % remaining |
|---|---|---|
| Putty Formulation | at 72 hrs. | at 72 hours |
| Orthostat-L (Control) | 84 | 16 |
| 60% CS/19% Sorb. Monooleate/5% | 60 | 40 |
| TA/16% LFB | | |
| 55% CS/29% TA/16% LFB | 40 | 60 |
| 55% CS/29% SAIB/16% LFB | 29 | 71 |
| 55% CS/29% Toc. Succinate/16% LFB | 63 | 37 |
| 55% CS/24% L-121/5% TA/16% LFB | 61 | 39 |
| 57.5% CS/5% VK 1/22.5% TEC/15% LFB | 75 | 25 |

Table 4.2 summarizes the release kinetics profile of formulations containing the reservoir Pluronic L-121 combined with one or more additional reservoirs. The addition of secondary reservoirs to the primary reservoir, L121, may act to decrease or increase % Drug Released at 72 hours.

TABLE 4.2

| | % Drug | Drug Release Rate (mg/ml) at Specific | | | | |
|---|---|---|---|---|---|---|
| | Released | Time Points (hours) | | | | Time to Rate |
| Putty Formulation | at 72 hours | 1 | 24 | 48 | 72 | of 1.8 mg/hr |

Control

| | | | | | | |
|---|---|---|---|---|---|---|
| Orthostat-L (55% CS/24% L-35/5% TA/16% LFB) | 84% | 22.5 | 3.3 | 1.7 | 0.8 | 46 |

L-121 + TA Formulations

| | | | | | | |
|---|---|---|---|---|---|---|
| 55% CS/24% L-121/5% TA/16% LFB | 61% | 19.0 | 2.2 | 1.1 | 0.8 | 32 |
| 55% CS/21.5% L-121/7.5% TA/16% LFB | 68% | 20.6 | 2.7 | 1.3 | 0.8 | 36 |
| 55% CS/19% L-121/10% TA/16% LFB | 63% | 18.1 | 2.2 | 1.2 | 0.8 | 34 |

L121 + TS Formulations

| | | | | | | |
|---|---|---|---|---|---|---|
| 55% CS/24% L-121/5% TS/16% LFB | 78% | 17.8 | 2.4 | 1.3 | 0.9 | 37 |
| 55% CS/19% L-121/10% TS/16% LFB | 67% | 17.0 | 2.2 | 1.3 | 1.2 | 35 |
| 55% CS/14% L-121/15% TS/16% LFB | 71% | 14.8 | 2.2 | 1.2 | 0.8 | 34 |
| 53% CS/13% L-121/14% TS/20% LFB | 60% | 20.3 | 2.8 | 1.5 | 1.1 | 43 |
| 52% CS/12% L-121/12% TS/24% LFB | 59% | 21.9 | 3.4 | 1.8 | 1.3 | 48 |

L121 + TA + TPGS Formulations

| | | | | | | |
|---|---|---|---|---|---|---|
| 55% CS/9% L-121/10% TA/10% TPGS/16% LFB | 91% | 22.4 | 4.1 | 1.9 | 1.0 | 51 |
| 45% CS/10% CA/9% L-121/15% TA/5% TPGS/ | 91% | 14.3 | 7.6 | 0.1 | 0.1 | 42 |
| 16% LFB | | | | | | |
| 55% CS/9% L121/15% TA/5% TPGS/16% LFB | 87% | 20.2 | 3.8 | 1.7 | 0.9 | 47 |

L121 + TS + TPGS Formulation

| | | | | | | |
|---|---|---|---|---|---|---|
| 55% CS/9% L-121/15% TS/5% TPGS/16% LFB | 71% | 15.6 | 2.5 | 1.4 | 0.9 | 40 |

L121 + TA + TS Formulation

| | | | | | | |
|---|---|---|---|---|---|---|
| 55% CS/19% L-121/5% TA/5% TS/16% LFB | 56% | 15.8 | 2.0 | 1.1 | 0.7 | 30 |

Table 4.3 summarizes the release kinetics profile for a single reservoir system without the use of additional vehicles. Each reservoir has differential effect on the release kinetics of the drug.

TABLE 4.3

| | | Release Rate (mg/hr | | | | Time to |
|---|---|---|---|---|---|---|
| | % released | 1 | 24 | 48 | 72 | LFB RR |
| Putty Formulation | at 72 hrs. | hr | hrs | hrs. | hrs. | 1.8 mg/hr |
| 55% CS/29% SAIB/ | 29% | 6.8 | 1.1 | 0.7 | 0.5 | 12 |
| 16% LFB | | | | | | |
| 55% CS/29% TA/ | 40% | 10.3 | 1.4 | 0.8 | 0.5 | 20 |
| 16% LFB | | | | | | |
| 55% CS/29% TS/ | 63% | 12.2 | 2.6 | 1.4 | 0.7 | 39 |
| 16% LFB | | | | | | |
| 55% CS/29% TPGS/ | 92% | 31.2 | 4.2 | 0.5 | 0 | 40 |
| 16% LFB | | | | | | |

RR = release rate; CS = calcum stearate; TA = tocopherol acetate; LFB = lidocaine free base; TPGS = tocopherol polyethylene glycol succinate; SAIB = Sucrose Acetate Isobutyrate; TS = tocopherol succinate

### Adjustment of Elution Kinetics Through the Use of Kinetics Modifiers

### A. Varying the Amounts of Dl α-Tocopherol Acetate.

This example demonstrates the alteration of lidocaine free base elution kinetics by varying the proportion of reservoir to the other components of the formulation. In this example, dl-α-tocopherol acetate is used as the reservoir. Four different formulations were prepared keeping the weight percentages of calcium stearate and lidocaine free base constant at 55% and 16% respectively while varying the relative amounts of Pluronic L-35 and dl-α-tocopheryl acetate. The amount of lidocaine eluting over 72 hours was measured. For each formulation the results are given in Table 4.4 below and represented in FIG. 2.

TABLE 4.4

| | | | | | | Time to |
|---|---|---|---|---|---|---|
| | | | | | | LFB RR |
| | % at | RR @ | RR @ | RR@ | RR @ | 1.8 mg/hr |
| Putty Formulation | 72 hrs. | 1 hr. | 24 hrs. | 48 hrs. | 72 hrs. | (hours) |
| Orthostat-L (55% CS/24% L-35/5% TA/16% LFB) | 84% | 22.5 | 3.3 | 1.7 | 0.8 | 46 |
| 55% CS/19% L-35/10% TA/16% LFB | 74% | 21.0 | 2.6 | 1.5 | 1.0 | 42 |
| 55% CS/14% L-35/15% TA/16% LFB | 60% | 18.0 | 2.0 | 1.3 | 0.9 | 32 |
| 55% CS/9% L-35/20% TA/16% LFB | 55% | 17.6 | 1.8 | 1.1 | 0.8 | 24 |

L-35 = Pluronic L-35. Other abbreviations as in Table 4.3

Table 4.5 summarizes the release kinetics profile for compositions containing 6% lidocaine free base.

TABLE 4.5

| | % Drug | | | | | Time |
|---|---|---|---|---|---|---|
| | released | RR @ | RR @ | RR @ | RR @ | when RR |
| Putty Formulations | @ 72 hrs. | 1 hr. | 24 hrs. | 48 hrs. | 72 hrs. | 1.8 mg/hr |
| 55% CS/0% L-35/39% TA/6% LFB | 37% | 4.5 | 0.4 | 0.3 | 0.2 | 1.5 |
| 55% CS/19% L-35/20% TA/6% LFB | 54% | 5.1 | 0.7 | 0.4 | 0.3 | 5 |
| 55% CS/24% L-35/15% TA/6% LFB | 74% | 9.4 | 1.0 | 0.5 | 0.3 | 6 |
| 50% CS/29% L-35/10% TA/6% LFB | 90% | 10.7 | 1.3 | 0.5 | 0.2 | 18 |
| 55% CS/34% L-35/5% TA/6% LFB | 96% | 14.1 | 1.6 | 0.5 | 0.1 | 3 |

### B. Changing the Reservoir—Vitamin K

This example demonstrates the role of changing the reservoir in altering the elution kinetics of the therapeutic agent, lidocaine free base. The reservoir, dl-α tocopherol acetate, was substituted with another reservoir, vitamin K, in the same percentage. This change resulted in an alteration of the cumulative percentage of lidocaine eluted at 72 hours. Other examples of alternate reservoirs are presented in Table 4.1 & 4.2.

TABLE 4.6

| | | | | Lidocaine |
|---|---|---|---|---|
| | | | | Eluted- |
| Solid | Modifier | Reservoir | Drug | 72 Hrs |
| 57.5% | 16.5% | 10% TOC | 16% | 61% |
| Calcium | Triethyl | acetate | lidocaine | |
| Stearate | Citrate | | free base | |
| 60% | 14% | 10% | 16% | 46% |
| Calcium | Triethyl | Vitamin | lidocaine | |
| Stearate | Citrate | K1 | free base | |
| 50% | 24% | 10% | 16% | |
| Calcium | Pluronic | cholesterol | lidocaine | |
| stearate | L-101 | | free base | |
| 50% | 21% | 13% | 16% | |
| Calcium | Pluronic | cholesterol | lidocaine | |
| Laurate | L-101 | | free base | |

### C. Ratios of Anesthetic Salt Forms

This example demonstrates the effect of using the different salt forms and eutectic complexes of the therapeutic agent in altering the elution kinetics of the therapeutic agents. The examples in Table 4.7 are embodiments in which the total percentage of the therapeutic agent was kept constant but a portion of the free base version of the therapeutic agent was substituted with the salt form. This substitution produced an alteration of the cumulative percentage of lidocaine eluted at 72 hours.

TABLE 4.7

| | | | | | | Time to |
|---|---|---|---|---|---|---|
| | % released | RR @ | RR@ | RR@ | RR @ | LFB RR |
| Putty Formulation | @ 72 hrs. | 1 hr. | 24 hrs. | 48 hrs. | 72 hrs. | 1.8 mg/hr |
| 57.5% CS/16.5% TEC/10% | 61% | 32.8 | 11.0 | 3.7 | 1.9 | 1.9 @ |
| TA/16% LFB | | | | | | 72 hrs |
| 57.5% CS/16.5% TEC/10% | 70% | 27.5 | 0.9 | 0.7 | 1.2 | NA |
| TA/10% LFB/6% LHCl | | | | | | |

TEC: triethyl citrate; LHCL = Lidocaine hydrochloride

In Table 4.8, the effects of replacing the free base version of the drug with eutectic complexes of the drug and other agents are demonstrated. Increasing the concentration of the reservoir in the drug delivery system decreases cumulative lidocaine eluted. In one embodiment, lauric acid is combined with lidocaine free base to create a eutectic liquid at room temperature. In a different embodiment, lidocaine free base is combined with tocopherol succinate to create a eutectic liquid at room temperature. In both embodiments the eutectic liquid is released from the reservoir in a predictable and controlled manner.

TABLE 4.8

| | | | | | | |
|---|---|---|---|---|---|---|
| | | | | | | Time to |
| | % at | R. Rate | R. Rate | R. Rate | R. Rate | LFB RR |
| Putty Formulation | 72 hrs. | @ 1 hr. | @ 24 hrs. | @ 48 hrs. | @ 72 hrs. | 1.8 mg/hr |

Control

| | | | | | | |
|---|---|---|---|---|---|---|
| Orthostat-L (55% CS/24% L-35/ | 84% | 22.5 | 3.3 | 1.7 | 0.8 | 46 |
| 5% TA/16% LFB) | | | | | | |

Eutectics

| | | | | | | |
|---|---|---|---|---|---|---|
| 55% CS/16% L-35/5% TA/8% | 85% | 24.8 | 3.0 | 1.3 | 0.6 | 41 |
| LFB/16% L. Acid + LFB | | | | | | |
| Eutectic | | | | | | |
| 38% CS/43% TS/19% LFB | 65% | 17.3 | 3.4 | 1.7 | 0.7 | 47 |

### D. Adjustment of the Elution Kinetics by Changing the Vehicle

This example demonstrates the role of changing the vehicle in altering the elution kinetics of the therapeutic agent, lidocaine free base. The kinetics modifier, triethyl citrate, was substituted with Pluronic L-35. This substitution produced an alteration of the cumulative percentage of lidocaine eluted at 72 hours (Table 4.9).

TABLE 4.9

| | | | | Lidocaine |
|---|---|---|---|---|
| | | | | Eluted |
| Solid | Modifier | Reservoir | Drug | @ 72 hrs |
| 62% | 12% | 5% TOC | 16% | 60% |
| Calcium | Triethyl | acetate | lidocaine | |
| Laurate | Citrate | | free base | |
| 62.5% | 11.5% | 5% TOC | 16% | 39% |
| Calcium | Pluronic ® | acetate | lidocaine | |
| Laurate | L-35 | | free base | |

### E. Changing the Metal Salt Form of a Fatty Acid.

This example demonstrates the effect of varying the stearate salt form on lidocaine elution. Three different formulations were prepared keeping the weight percentages of Tocopherol acetate (reservoir) and lidocaine free base constant at 5% and 16% at respectively while replacing calcium stearate with the indicated alternative stearate salt form. The weight of Pluronic, L-35 (modifier) and stearate were adjusted in order to form acceptable handling putty. The amount of lidocaine eluting over 72 hours was measured. For each formulation the results are given in Table 4.10 below.

TABLE 4.10

| | | | | Lidocaine |
|---|---|---|---|---|
| | | | Lidocaine | Eluted - |
| Solid | Modifier | Reservoir | Free Base | 72 hrs |
| 60% Zinc Stearate | 19% | 5% | 16% | 48% |
| 60% Magnesium | 19% | 5% | 16% | 39% |
| Stearate | | | | |

### F. Incorporation of Solid Dispersants

Table 4.11 summarizes the release kinetics for formulations containing micronized solid dispersants. The dispersants used include Kollidon CL-M(CL-M), Glycerol Phosphate Calcium Salt (GPCS) and Calcium Alginate (CA).

TABLE 4.11

| | | | | | | Time to |
|---|---|---|---|---|---|---|
| | % at | RR @ | RR@ | RR@ | RR @ | LFB RR |
| Putty Formulation | 72 hrs. | 1 hr. | 24 hrs. | 48 hrs. | 72 hrs. | 1.8 mg/hr |
| 40% CS/15% CL-M/29% TA/16% | 66% | 13.8 | 5.9 | 1.4 | 1.1 | 42 |
| 25% CS/34% GPCS/25% TA/16% LFB | 54% | 15.5 | 6.2 | 1.3 | 1.0 | 41 |
| 25% CA/30% CS/29% TA/16% LFB | 92% | 16.7 | 4.5 | 2.8 | 0.5 | 56 |

### Example 5

### Adjusting Absorption Rate by Changing Solubility of the Solid

### Chain Length

This example demonstrates the role of changing the chain length of the fatty acid in altering the solubility of the solid. The solubility of the calcium salts of palmitic acid, lauric acid and oleic acid are known in the art and are, dependent upon alkyl chain length and degree of substitution, soluble in both water and oleic acid. Their relative solubilites were obtained from the literature (Jandacek 1991) and are described in Table 5.1.

TABLE 5.1

| | | | | Solubility of |
|---|---|---|---|---|
| | | | | Solid in Oleic |
| Solid | Vehicle | Modifier | Drug | Acid @ 40° C. |
| 60% | 19% | 5% TOC | 16% | 15.6% |
| Calcium | Triethyl | acetate | lidocaine | |
| Palmitate | Citrate | | free base | |
| 60% | 19% | 5% TOC | 16% | 22.8% |
| Calcium | Pluronic | acetate | lidocaine | |
| Laurate | L-35 | | free base | |
| 60% | 19% | 5% TOC | 16% | 53.3% |
| Calcium | Pluronic | acetate | lidocaine | |
| Oleate | L-35 | | free base | |

Formulations were created containing either calcium laurate or calcium stearate. The amount of drug added was kept constant and the liquid vehicles and reservoirs were adjusted to achieve consistent handling properties. Formulations A and B contained calcium stearate and formulations C and D contained calcium laurate. The complete formulations are shown in Table 5.2 below.

TABLE 5.2

| Sample | |
|---|---|
| Names | Formulations |
| A | Calcium Stearate 55%; TA 5%; PLU 24%; LFB 16% |
| B | Calcium Stearate 50%; TA 5%; PLU35 14.5%; PLU68 |
| | 14.5%; LFB 16 |
| C | Calcium Laurate 67%; TA 5%; TEC 12%; LFB 16% |
| D | Calcium Laurate 62.5%; TA 7.5%; PLU35 14%; LFB 16% |

To test the effect of chain length on absorption, a sheep tibial defect model was used. A cranialmedial skin incision was made approximately 5 to 8 cm from the stifle joint, parallel to the tibial crest, to the mid-diaphysis. Subcutaneous fascia was cut longitudinally to expose the tibia and retracted with self retaining retractors. A freer elevator was used to elevate the periosteum. A Stryker oscillating saw with two 1 mm blades attached (1×4 mm) was used to create ˜2 mm×7 mm slot defects in the tibia bone (diaphyseal region). A total of 3 slot defects were created in each of the tibiae per animal (6 total slot defects per animal). The defect sites were cleared of bone fragments prior to application of the test article(s). The test article(s) were applied to the created slot defects sufficient to fill the defect site. Animals were scarificed 4 weeks after surgery and implants were evaluated histologically.

The product absorption was scored on a 3 point scale. 0 indicates no absorption and 2 indicates complete absorption. The results are shown in FIG. 3. Formulations A and B which contained the longer chain fatty acid salt absorbs at a slower rate than formulations C and D.

### Example 6

### Adjusting Absorption Rates Using Erodible Carrier Combinations

The formulations listed in Table 6.1 include the usage of two solids to alter the absorption rate. In the examples, solid 2 consists of various erodible carriers.

TABLE 6,1

| Solid1 | Solid2 | Vehicle | Modifier | Drug |
|---|---|---|---|---|
| 30% | 25% | 24% | 5% TOC | 16% |
| Calcium | Poly(lactic | Pluronic | acetate | Lidocaine |
| Stearate | acid) | L-35 | | free base |
| 30% | 25% copolymer | 24% | 5% TOC | 16% |
| Calcium | of polyhydroxy- | Pluronic | acetate | Lidocaine |
| Stearate | butyrate and | L-35 | | free base |
| | polyhydroxy- | | | |
| | valerate | | | |
| 30% | 25% | 24% | 5% TOC | 16% |
| Calcium | Poly(SA-HAD | Pluronic | acetate | Lidocaine |
| Stearate | anhydride) | L-35 | | free base |

### Example 7

### Adjusting Absorption Rate Use of Dispersing Agents

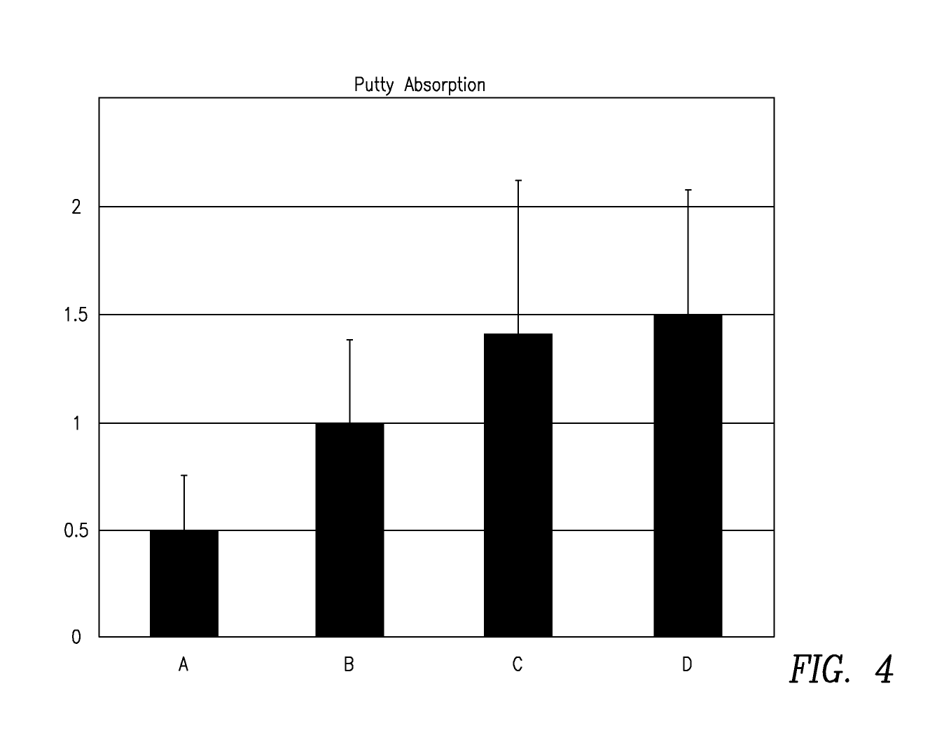

The formulations listed in Table 7.1 below include the usage of two solids to alter the absorption rate. In the examples, solid 2 consists of various dispersing agents. The formulations were tested for absorption in the sheep tibial slot defect model described in Example 5. In vivo absorption results at four weeks are presented in FIG. 4. The use of secondary solids as dispersing agents caused a change in the rate of putty absorption.

TABLE 7.1

| | % Calcium | | | | Drug |
|---|---|---|---|---|---|
| Sam- | Stearate | % | | % | % Lidocaine |
| ple | (Solid 1) | Solid 2 | Vehicle | Reservoir | free base |
| A | 55 | | 24% | 5% TOC | 16% |
| Con- | | | Pluronic | acetate | |
| trol | | | L-35 | | |
| B | 31 | 31% | 17% | 5% TOC | 16% |
| | | GPCS | Triethyl | acetate | |
| | | | Citrate | | |
| C | 45 | 18% | 19% | 5% TOC | 16% |
| | | Phosphatydil- | Pluronic | acetate | |
| | | choline | L-35 | | |
| D | 25% | 35% | 15% | 10% TOC | 16% |
| | | GPCS | Pluronic | acetate | |
| | | | L-35 | | |

### Example 8

### Adjusting Absorption Rate Using Water Soluble Solids

The formulations listed in Table 8.1 include the usage of water soluble, solids to alter the absorption rate. In the examples, the vehicle can consist of a single water soluble solid or combinations of water soluble solids and water soluble modifiers.

TABLE 8.1

| | | | Water | Water | | |
|---|---|---|---|---|---|---|
| | Insoluble | Insoluble | soluble | soluble | | |
| Formulation | Solid1 | Solid 2 | solid | modifier | Modifier | Drug |
| A | 55% | | | 24% | 5% TOC | 16% Lidocaine |
| (Control) | Calcium | | | Pluronic | acetate | free base |
| | Stearate | | | L-35 | | |
| B | 45% | | 8% | 16% | 10% TOC | 16% Lidocaine |
| | Tricalcium | | PEG2000 | PEG900 | acetate | free base |
| | Phosphate | | | | | |
| C | 13% | | 62% | | 11% TOC | 14% Lidocaine |
| | Calcium | | PEG2000 | | acetate | free base |
| | Stearate | | | | | |
| D | 40% | 10% | 19% | 10% | 5% TOC | 16% Lidocaine |
| | Tricalcium | Calcium | Pluronic | Pluronic | acetate | free base |
| | Phosphate | Stearate | F-68 | P-123 | | |

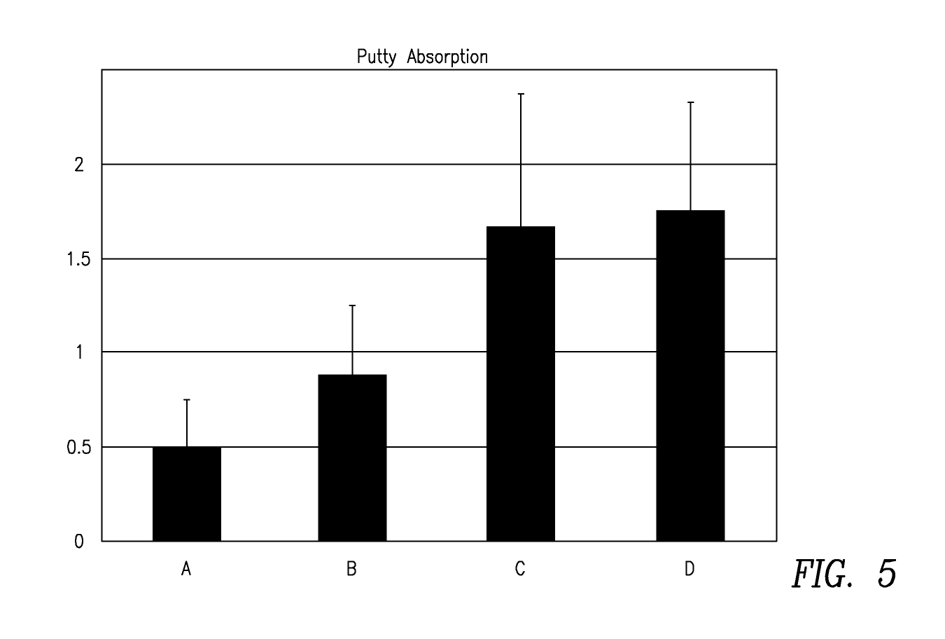

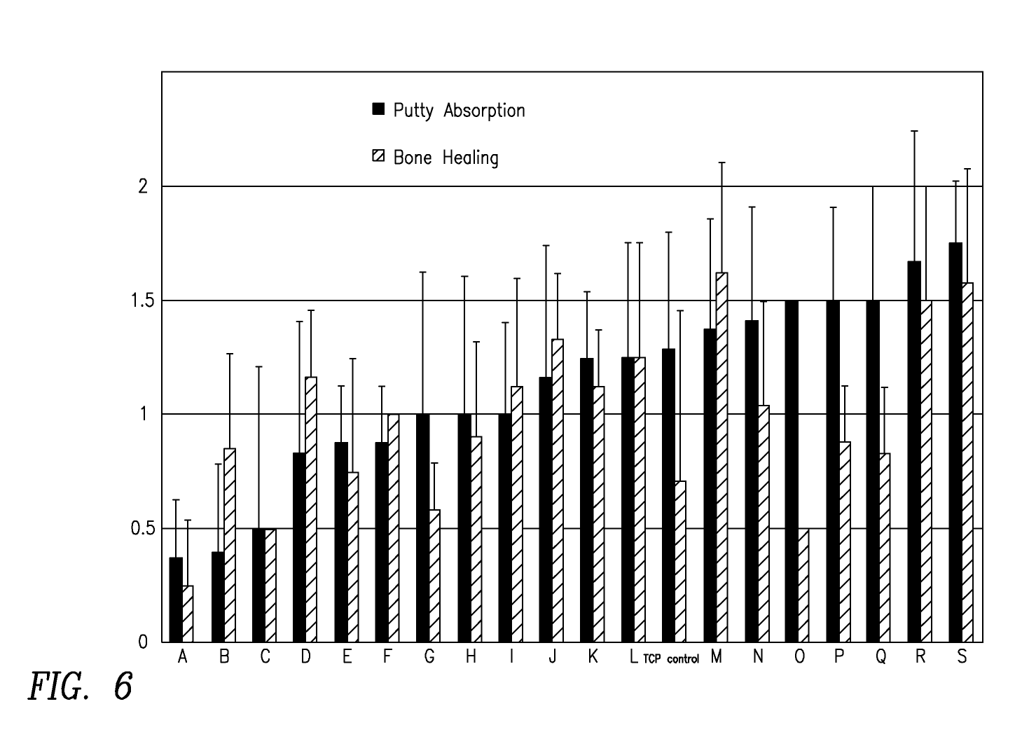

The formulations were tested for absorption in the sheep tibial slot defect model described in Example 5. In vivo absorption results at four weeks are presented in FIG. 5. The addition of water soluble solids increased the absorption rate over the control formulation (A) which had a higher proportion of insoluble solid. FIG. 6 demonstrates in vivo absorption and bone healing results at four weeks for formulations shown in Table 8.2.

TABLE 8.2

| Sample | |
|---|---|
| ID | Formulation |
| A | Calcium Stearate 40%; CL-M 15%; TA 10%; PLU35 |
| | 19%; LFB 16% |
| B | ZnSt 60%; TA 5%; PLU35 19%; LFB 16% |
| C | Calcium Stearate 55%; TA 5%; PLU 24%; LFB 16% |
| D | Calcium Stearate 35%; CaSucc 31%; TA 5%; TEC |
| | 13%; LFB 16% |
| E | TCP 45%; Calcium Stearate 10%; TA 5%; PEG2000 |
| | 8%; PEG1000 16%; LFB 16% |
| F | Calcium Stearate 50%; TA 5%; PLU35 14.5%; PLU68 |
| | 14.5%; LFB 16 |
| G | TCP 40%; Calcium Stearate 20%; TA 5%; PLU35 19%; |
| | LFB 16% |
| H | Calcium Stearate 31%; GPCS 31%; TA5%; TEC 17%; |
| | LFB 16% |
| I | Calcium Stearate 60%; TA 5%; Nonanol 19%; LFB 16% |
| J | Calcium Stearate 22%; CaSach 10%; TCP 34%; TA 5%, |
| | TEC 13%; LFB 16% |
| K | Calcium Laurate 67%; TA 5%; TEC 12%; LFB 16% |
| L | Calcium Laurate 62.5%; TA 7.5%; PLU35 14%; LFB 16% |
| M | TCP 50%; TA 10%; PLU68 16%; PLU123 8%; LFB 16% |
| N | Calcium Stearate 45%; PC 18% TA 10%; PLU35 12%; |
| | LFB 15% |
| O | TCP 40%; Calcium Stearate 10%; TA 15%; PLU68 14%; |
| | PLU123 5%; LFB 16% |
| P | Calcium Stearate 29.8%; GPCS 29.5%; TA 9.55; PLU35 |
| | 16.2%; LFB 15% |
| Q | TCP 40%; Calcium Stearate 10%; Vit K 5%; PLU68 19%; |
| | PLU123 10%; LFB 16% |
| R | Calcium Stearate 13.3%; TA 11.1%; PEG2000 62.3%; |
| | LFB 13.3% |
| S | TCP 40%; Calcium Stearate 10%; TA 5%; PLU68 19%; |
| | PLU123 10%; LFB 16% |

*TCP control is B-TCP from Berkeley Advanced Biomaterials, Inc. Product # BABI-TCP-G5

The product absorption was scored on a 3 point scale. 0 indicates no absorption and 2 indicates complete absorption. Bone healing was also scored on a 3 point scale. 0 indicates no bone growth and 2 indicates complete filling of the defect with newly formed bone.

### Example 9

### Testing Analgesia in a Rabbit Bone Pain Model

This example describes an animal model used in the evaluation of the inventive putties in alleviating pain originating from a bone defect.

### Surgical Procedure

An incision is made in the medial aspect of the rabbit's left hind leg over the tibia. The skin is retracted laterally. The periosteum is split to provide access to the bone. A single defect (4 mm diameter by approximately 5 mm deep) is created with a manual surgical drill in the tibia. The periosteum surrounding the defect is disrupted.

### Analgesia Evaluation

The animals are allowed to recover from anesthesia. No post-operative analgesia is administered. At 5, 24, 48 and 72 hours post-surgery the animals are observed for expression of pain as per the evaluation criteria listed below. The rabbits receiving the placebo are compared to the rabbits receiving the anesthetic matrix.

TABLE 9.1

| | Observation | Rating |
|---|---|---|
| 1 | Weight Bearing | 2 = continuous weight bearing |
| | | 1 = intermittent weight bearing |
| | | 0 = completely non weight bearing |
| 2 | Gait with Movement | 2 = continuous |
| | | 1 = intermittent |
| | | 0 = non-weight bearing |
| 3 | Withdrawal Reflex | 3 = absent |
| | (Painful stimuli is | 2 = present with hard pinching |
| | applied at and near | 1 = present with mild pinching |
| | the bone defect site) | 0 = present |
| 4 | Synchronous Hopping | 2 = mostly in synch |
| | with both legs (refers | 1 = mostly out of synch |
| | only to hopping) | 0 = always out of synch |
| 5 | Rests on toe when | 2 = frequently |
| | stationary (surgical | 1 = once in a while |
| | side) | 0 = Never |
| 6 | Foot slips while | 2 = frequently |
| | walking or hopping | 1 = some |
| | (surgical side) | 0 = never |
| 7 | Animal appears to | 2 = no |
| | have full control | 1 = so/so |
| | over leg movement | 0 = yes |

Individual animals are scored according to the above scale. Items 1-3 are most useful in scoring a simple analgesic effect. Items 4-7 are useful to demonstrate release of analgesic when there is a significant motor block component to the behavior. In such instances the motor block may be confused with the limping response. In such instances, items 4-7 produce a score verifying release of therapeutic levels of anesthetic, but incorporate and evaluation of the motor block component as well.

### Example 10

### Rat Sciatic Nerve Block Model

### Subfascial Sciatic-Nerve Implantations

For subfascial sciatic nerve implantation of an anesthetic matrix of the invention, rats are anesthetized initially by brief inhalation of 2% sevoflurane followed by intraperitoneal injection of 50 mg/kg pentobarbital Na (Nembutal®), and the sciatic nerve is exposed by lateral incision of the thigh and blunt division of the superficial fascia and muscle. A drug carrier formulation containing 16% Lidocaine Free base as described in example 1, Table 1.2, was shaped into a cylinder (˜1.25×0.25 cm), and is also placed underneath the fascia next to the sciatic nerve. The superficial muscle layer is closed with 4-0 Vicryl sutures placed approximately 3 mm apart to minimize displacement of the anesthetic matrix and the skin incision is closed with 4-0 Prolene sutures.

### Neurobehavioral Examination

Initially, the rats are examined before implantation of the anesthetic matrix and then at 30 and 60 minutes and 3, 6, 12 hours after implantation and then daily until complete functional recovery is established.

Motor function is assayed by holding the rat upright with the control hind limb extended so that the distal metatarsus and toes of the target leg supported the animal's weight; the extensor postural thrust is recorded as the force (in grams) applied by each of the 2 hind limbs to a digital platform balance (Ohaus Lopro; Fisher Scientific, Florham Park, N.J.). The reduction in this force, representing reduced extensor-muscle contraction caused by motor block, is calculated as a percentage of the control force (preinjection control-value range was 145 to 165 g). The obtained percentage value is assigned a ‘range’ score: 0=no block or baseline; 1=minimal block, force between preinjection control value of 100% and 50%; 2=moderate block, force between 50% of the preinjection control value and 20 g (˜20 g represented the approximate weight of the flaccid limb); 3=complete block, force 20 g or less.

Nociception is evaluated by the withdrawal reflex motion and vocalization to pinch of a skin fold over the lateral metatarsus (cutaneous pain) and of the distal phalanx of the fifth toe (deep pain). We grade the combination of nocifensive withdrawal reflex and vocalization on a scale of 0 to 3 for each examination, and as with the motor assessment, we repeat the examination three times in each trial, reporting an average of the three exams. Grading is scored on a scale of 0-3, as follows: 3=complete block, no nocifensive reaction or vocalization is observed; 2=moderate block, vocalization accompanied by slow withdrawal and flexion of the leg. 1=minimal block, brisk flexion of the leg, with some sideways movement of the body or other escape response and loud vocalization. 0 indicates the baseline where no block is present and all the nocifensive responses just listed are detected

For evaluation of dose-dependent effects among different groups, the complete-block times (CBT), defined as the time from injection/implantation to the first signs of recovery (above 25% of normal force [=20 g] are counted as a sign of recovery of motor block and any nocifensive reaction to pinch is counted as a sign of recovery of nociceptive block) and the complete-recovery time (CRT), defined as the time from implantation to the time of complete recovery of function.

### Exemplary Results Shown in:

Wang C F, Djalali A G, Gandhi A, Knaack D, De Girolami U, Strichartz G, Gerner P. An absorbable local anesthetic matrix provides several days of functional sciatic nerve blockade. Anesth Analg. 2009 March; 108(3):1027-33.

### Example 11

### Topical Anesthetic

An anesthetic delivery putty of the invention is prepared as described in Examples 1 above (first example Table 1.2) and is formed into disk shape approximately 2 mm thick and 2 cm in diameter. The putty is placed against the skin of two subjects and covered with either an adhesive pad or a band aid. Periodically the putty and adhesive are temporarily removed and the surface of the skin probed with a pin for sensitivity to pain. At 48 and 72 hours the surface of the skin is probed with the pin to test for decreased sensitivity to pain.

Drug delivery putties of the invention appropriate for topical administration of anesthetic include those that are formulated with a hydrophobic liquid vehicle such as hydrophobic Pluronics, decanol or isopropyl myristate comprising between one and forty percent of the putty by mass, an anesthetic comprising up to 20% of the putty by mass and a solid vehicle such as calcium stearate, calcium laurate or high MW polyethylene glycol comprising up to 70% of the putty by mass. Preferred embodiments include agents to promote penetration of the anesthetic through the stratum corneum. One penetration enhancement strategy is to prepare a fatty acid salt of an anesthetic freebase, such as bupivicaine or lidocaine laurate.

### Example 12

### Delivery of Statins

A variety of animal studies have shown that local delivery of Lovastatin to bony sites may stimulate fracture healing. Lovastatin was incorporated into a drug delivery putty of the invention and kinetics of Lovastatin elution were determined

TABLE 12.1
Putty Composition

| Component | % Composition |
|---|---|
| Calcium Stearate | 54 |
| Triethyl Citrate | 36 |
| Lovastatin | 10 |

### Procedure

0.5 g of the putty were prepared as follows:

0.05 g of Lovastatin (Sigma) was placed into a glass beaker. 0.18 g of triethyl citrate was weighed into beaker. The mixture was heated until the Lovastatin dissolved into the triethyl citrate forming a homogeneous solution. 0.27 g of calcium stearate was then added to the beaker and the components were mixed until dry calcium stearate was no longer visible and granules formed. The granules were then removed from the beaker and molded into a coherent putty.

The putty containing Lovastatin was prepared, divided into 50 mg units, and put into 1 L glass jars for elution. Putty samples were removed from the elution jars at the indicated times, lightly patted dry with a Kimwipe and saved for Lovastatin analysis.

Lovastatin was extracted at room temperature from putty samples in a test tube by adding isopropanol to the putty in a ratio of 20 mg of putty to 1 mL of isopropanol and vortexing three times over the course of seven minutes. The solution in each test tube was transferred to individually marked microcentrifuge tubes and centrifuged at 13,000 rpm for fifteen minutes. 1 mL of the supernatant was removed via pipette and transferred into a clean test tube. The supernatant was diluted 1:100 in Isopropyl alcohol and read at 247 nm.

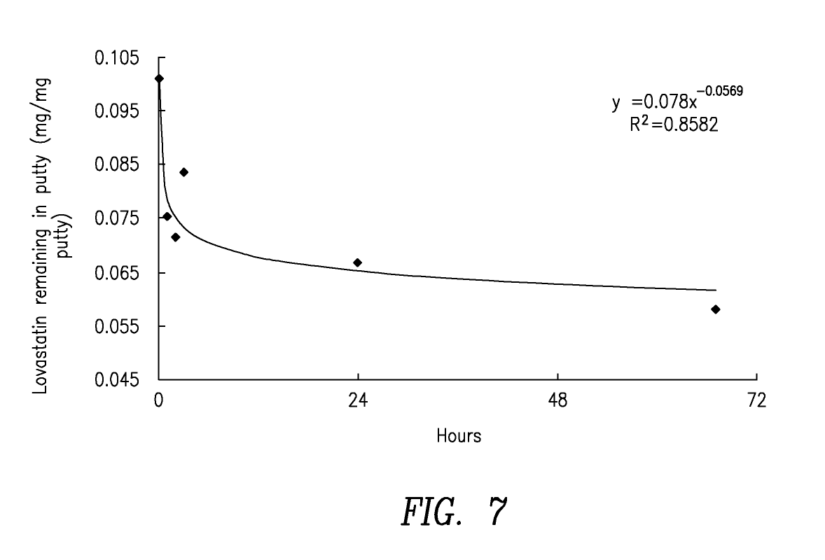

Lovastatin concentrations were determined spectrophotometrically using a standard curve prepared by determining absorbance of serial dilutions of lovastatin in isopropanol. FIG. 7 shows the amount of Lovastatin remaining in samples (mg/mg putty) over a 67-hour period.

A variety of animal studies have shown that local delivery of Lovastatin to bony sites may stimulate fracture healing. Lovastatin was incorporated into the drug delivery device to determine Lovastatin elution kinetics.

TABLE 12.2
Putty Composition

| Component | % Composition |
|---|---|
| Calcium Stearate | 54 |
| Triethyl Citrate | 36 |
| Lovastatin | 10 |

### Procedure

The statin-based delivery system was prepared as follows:
0.05 g of Lovastatin (Sigma Aldrich) was placed into a glass beaker
0.18 g of Triethyl Citrate was weighed into beaker
The mixture was heated until the Lovastatin dissolved into the Triethyl citrate forming a homogeneous solution
0.27 g of Calcium Stearate was then added to the beaker
The components were mixed until dry calcium stearate was no longer visible and putty aggregates formed
The aggregates were then removed from the beaker and molded into coherent putty
   The Lovastatin putty was divided into 50 mg units and put into 1 L glass jars for extraction analysis. The samples were removed from the elution solutions at the indicated times, lightly patted dry with a Kimwipe and saved for Lovastatin analysis.
   Lovastatin was extracted from putty samples in a test tube by adding isopropanol to the putty in a ratio of 20 mg of putty to 1 mL of isopropanol. The test tube was then vortexed three times over a seven minute period at room temperature. The solution in each test tube was transferred to individually marked microcentrifuge tubes and centrifuged at 13,000 rpm for fifteen minutes. 1 mL of the supernatant was removed via pipette and transferred into a clean test tube. The supernatant was diluted 1:10 in Isopropyl alcohol and read at 247 nm. Lovastatin concentrations were determined spectrophotometrically using a standard curve prepared by determining absorbance of serial dilutions of Lovastatin in isopropanol. FIG. 7 shows the amount of Lovastatin remaining in samples (mg/mg putty) over a 67-hour period. In another preferred embodiment, a statin is mixed with the carrier of Example 26 at ratios (wt/wt) of 1 part statin to 99 parts carrier, 5 parts statin plus 95 parts carrier, 10 parts statin to 90 parts putty and 20 parts satin to 80 parts carrier.

### Example 13

### Delivery of Hydrophobic Anti Cancer Agents

This example demonstrates the incorporation of the hydrophobic, anti-cancer agent paclitaxel, into a drug delivery device of the invention (Table 13.1). Additional classes of anti-cancer agents that could be delivered from the invention include anti-angiogenic agents, anti-proliferative agents, chemotherapeutic agents and monoclonal antibodies.

TABLE 13.1

| Solid | Vehicle | Modifier | Drug |
|---|---|---|---|
| 55% | 24% | 5% TOC | 16% |
| Calcium | Pluronic | acetate | paclitaxel |
| Stearate | L-35 | | |

### Example 14

### Vaccine Delivery

This example demonstrates the incorporation of the vaccine diphtheria toxoid, into a drug delivery device of the invention. The drug delivery device of the invention is also able to deliver immunogenic hydrophobic complexes consisting of proteosomes, adjuvant compositions comprising at least one synthetic hydrophobic lipopolysaccharide or Pluronics, and other vaccine antigens including tetanus toxoid, and anthrax recombinant protective antigen.

TABLE 14.1

| Solid | Vehicle | Modifier | Drug |
|---|---|---|---|
| 60% | 25% | 5% TOC | 10% |
| Calcium | Pluronic | acetate | diphtheria |
| Stearate | L-35 | | toxoid |

### Example 15

### Use of Semi-Solid Malleable Vehicles to Decrease Cumulative Lidocaine Eluted

This example demonstrates the role of incorporating semi-solid malleable vehicles in altering the elution kinetics of the therapeutic agent, lidocaine free base. The percentages of all components were kept constant. The use of a single semi-solid malleable vehicle or multiple semi-solid malleable vehicles can alter the cumulative percentage of lidocaine eluted at 24 hours. Here the semi-solid malleable vehicles were a PEG2000/PEG900 blend and Pluronic P-103 and P-123 either alone or as a blend with other Pluronics. However, any vehicle with a wax-like consistency could be used.

TABLE 15.1

| | | | | | Lidocaine | |
|---|---|---|---|---|---|---|
| Solid | Vehicle1 | Vehicle2 | Modifier | Drug | Eluted - 24 hrs | Rates |
| 50% Tricalcium | 16% Pluronic | 8% Pluronic | 10% TOC | 16% lidocaine | 48% | |
| Phosphate | F-68 | P-123 | acetate | free base | | |
| 55% Calcium | 24% Pluronic | | 5% TOC | 16% lidocaine | | |
| Phosphate | P-103 | | acetate | free base | | |
| 50% Tricalcium | 12% PEG2000 | 12% PEG900 | 10% TOC | 16% lidocaine | 17% | |
| Phosphate | | | acetate | free base | | |

### Example 16

### Dual Phase Delivery System (Increased Capacity and/or Altered Elution Profile)

This example demonstrates the ability to increase the capacity of the drug delivery system and at the same time to alter the elution kinetics of the therapeutic agent. The example enables release to be dictated by two different components of the system. The method for formulating the drug delivery system is as follows:

Poly (D,L) lactide is dissolved in chloroform. The therapeutic drug is dissolved into the poly (D,L) lactide. The chloroform is allowed to evaporate to produce PLA encapsulated drug. The solid is micronized. The micronized solid is added to a formulation containing drug dissolved in the vehicle.

TABLE 16.1

| Solid1 | Solid2 | Vehicle | Modifier | Drug |
|---|---|---|---|---|
| 30% | 25% PLA | 24% | 5% TOC | 16% |
| Calcium | encapsulated | Pluronic ® | acetate | Lidocaine |
| Stearate | containing ~1 | L-35 | | free base |
| | to 75% drug | | | |

The poly (D,L) lactide acts as a slow release reservoir for drug delivery. The calcium stearate and Pluronic® provide for a faster release reservoir for drug delivery. Thus, by modulating how much drug (here, lidocaine) is dissolved in the poly (D,L) lactide and how much is suspended in the calcium stearate/Pluronic® the rate of release of drug can be modulated.

### Example 17

### Triple Phase Delivery System

This example demonstrates the ability to increase the capacity of the drug delivery system and at the same time to alter the elution kinetics of the therapeutic agent. The example enables release to be dictated by three different components of the system. The method for formulating the drug delivery system is as follows:

Poly (D,L) lactide is dissolved in chloroform. The therapeutic drug is dissolved into the poly (D,L) lactide. The chloroform is allowed to evaporate to produce PLA encapsulated drug. The solid PLA is micronized. A portion of the micronized PLA is gamma-irradiated. Gamma irradiation will increase the degradation rate of PLA and therefore the drug. The γ ray-irradiated PLA and non gamma-irradiated PLA is added to a formulation containing drug dissolved in the vehicle.

TABLE 17.1

| Solid1 | Solid2 | Solid3 | Vehicle | Modifier | Drug |
|---|---|---|---|---|---|
| 30% Calcium | 12% PLA | 12% (γ-irradiated) | 24% Pluronic ® | 5% TOC | 16% Lidocaine |
| Stearate | encapsulated | PLA encapsulated | L-35 | acetate | free base |
| | containing ~1 | containing ~1 | | | |
| | to 75% drug | to 75% drug | | | |

As above, the poly (D,L) lactide acts as a slow release reservoir for drug delivery. However, the γ ray-irradiated poly (D,L) lactide provides for a faster release than the unirradiated drug. The calcium stearate and Pluronic® provide for a shorter release-time reservoir for drug delivery. Thus, by modulating how much drug (here, lidocaine) is dissolved in the poly (D,L) lactide, how much is suspended in the γ ray-irradiated poly (D,L) lactide and how much is suspended in the calcium stearate/Pluronic® the rate of release of drug can be modulated.

### Example 18

### Increased Capacity for Drug Through Suspension of Drug in the Solid Phase of the Delivery Device

This example features the inclusion of a secondary agent depot within the drug delivery device of the invention. Specifically, in addition to the lidocaine provided by solubilizing lidocaine free base within the liquid, lidocaine was also suspended in the calcium phosphate solid phase. This demonstrates the ability to increase the capacity of the drug delivery system and at the same time to alter the elution kinetics of the therapeutic agent. The release profile is dictated by encapsulation of lidocaine within a settable calcium phosphate and dissolution of lidocaine into the vehicle incorporated into the putty. The following formulations were tested:

TABLE 18.1

| | | | | | | |
|---|---|---|---|---|---|---|
| Formulation | 40% settable | 10% | 19% | 10% | 5% alpha | 16% |
| #1 | Calcium | Calcium | Pluronic ® | Pluronic ® | Tocopherol | Lidocaine |
| | Phosphate | Stearate | F-68 | P123 | Acetate | |
| Formulation | 40% settable | 10% | 19% | 10% | 5% alpha | 16% |
| #2 | Calcium | Calcium | Pluronic ® | Pluronic ® | Tocopherol | Lidocaine |
| | Phosphate | Stearate | F-68 | P123 | Acetate | |
| | containing | | | | | |
| | 16% Lidocaine | | | | | |
| Formulation | 40% settable | 10% | 19% | 10% | 5% alpha | 16% |
| #3 | Calcium | Calcium | Pluronic ® | Pluronic ® | Tocopherol | Lidocaine |
| | Phosphate | Stearate | F-68 | P123 | Acetate | |
| | containing | | | | | |
| | 8% Lidocaine | | | | | |

### Procedure

The formulations described above were formulated as follows. For formulation 1, 3 ml of sterile water was added to 5 grams of Cem-Ostetic powder. For formulation 2, 480 mg of lidocaine was added to 3 ml of sterile water and 3 ml of sterile water containing lidocaine was added to 5 grams of Cem-Ostetic powder. For formulation 3, 290 mg of lidocaine was added to 3 ml of sterile water and 3 ml of sterile water containing lidocaine was added to 5 grams of Cem-Ostetic powder.

For all of the formulations, the mixture was allowed to harden for ˜30 minutes in a 45° C. oven. The hardened settable calcium phosphate was micronized using a mortar and pestle and was put through a sieve to ensure that the particle size was less than 300 microns.

For the preparation of the putty Pluronic® F-68, Pluronic® P 123, α-tocopherol acetate and lidocaine were mixed in the proportions listed above. The mixture was heated until the lidocaine was completely dissolved. The mixture was then added to the settable calcium phosphate and calcium stearate, described above, in the appropriate proportions. The components were mixed until the dry components had agglomerated. Then the agglomerated components were hand-molded into a coherent putty.

### Elution Testing

The samples were formed into disks (diameter—13.5 mm, thickness—2.5 mm) and placed in a nylon biopsy bag and sealed using a dialysis closure clip. The samples were placed in a VK 7000 dissolution bath containing 900 ml of 50 mM potassium phosphate buffered solution (pH=7.4) pre-warmed to 37° C. and set to a paddle speed of 25 RPM. At predetermined time intervals, 5 ml of the elution bath solution was collected for subsequent lidocaine free base detection and replaced with 5 ml of fresh solution.

Detection of lidocaine free base was assayed by the absorbance at 234 nm, using a UV/VIS Lambda 2 spectrophotometer. A calibration curve from standard solutions of lidocaine, also in KPO₄ buffer at pH 7.4, was used to calculate the percent lidocaine eluted at each time point and is shown as FIG. 2.

### Example 19

This example demonstrates the ability to increase the capacity of the drug delivery system and at the same time to alter the elution kinetics of the therapeutic agent by incorporation of a substantially anhydrous hydrogel-forming material into the carrier. The release profile is dictated by encapsulation and/or ionic interaction of the drug within the hydrogel-forming material (e.g. alginate or chitosan, other hydrogel-forming material which may be used include carboxymethylcellulose, carboxymethyl starch, oxidized cellulose, hypromellose and their derivatives.) and dissolution of lidocaine into the vehicle incorporated into the putty.

TABLE 19.1

| | | | | | | Time to |
|---|---|---|---|---|---|---|
| | % at | RR @ | RR @ | RR @ | RR @ | LFB RR |
| Putty Formulation | 72 hrs. | 1 hr. | 24 hrs. | 48 hrs. | 72 hrs. | 1.8 mg/hr |
| 25% CA/30% CS/29% TA/16% LFB | 92% | 16.7 | 4.5 | 2.8 | 0.5 | 59 |
| 55% CS/29% TA/16% LFB | 40% | 10.3 | 1.4 | 0.8 | 0.5 | 20 |

Procedure

Hydrogel was prepared as follows. 480 mg of lidocaine is added to 3 ml of water which was added to 5 grams of sodium alginate powder. An excess of calcium ions were added to facilitate cross linking of the hydrogel and swelling of the hydrogel with the lidocaine containing liquid. The hydrogel was made anhydrous by lyophilization, then, micronized with a mortar and pestle and passed through a sieve to ensure particle sizes were less than 300 micron.

The putty was prepared as follows. Pluronic® L35, α-tocopherol acetate and Lidocaine was added in the proportions listed above. The mixture was heated until the lidocaine is completely dissolved. The mixture is added to the hydrogel and calcium stearate in the appropriate proportions and was mixed until the dry components agglomerated. The agglomerated components are hand-molded into a coherent putty.

### Example 20

### Effect of Kinetic Modifiers with Different HLB Values

This example demonstrates how using alkaline oxide copolymers with varying hydrophilic to lipophilic balance (HLB) can predictably and controllably alter the rate of drug delivery. Specifically, alkylene oxide copolymers with HLB values ranging from 1 to 19, as shown in the table below, were used to create matrices for lidocaine delivery.

TABLE 20.1

| Pluronic name | Mean HLB |
|---|---|
| Pluronic L-101 | 1 |
| Pluronic L-121 | 1 |
| Pluronic L-62 | 7 |
| Pluronic 25R4 | 8 |
| Pluronic P-123 | 8 |
| Pluronic L-64 | 15 |
| Pluronic P-85 | 16 |
| Pluronic L-35 | 19 |

All formulations we prepared (wt %) 55% calcium stearate, 24% Pluronic, 5% tocopherol acetate and 16% lidocaine free base. The amounts of calcium stearate and tocopherol acetate were held constant to best demonstrate the effects of HLB on drug delivery (FIG. 8).

The release rate of lidocaine, expressed as time to release of 50% of the drug, (T1/2) was found to be inversely related to the HLB value of the putty. These results demonstrate that selection of a surfactant with a specific HLB value can be used to modify the burst release phase without disrupting the sustained release rate of the formulations. Table 20.2 further elaborates specific details of the sustained release phase of these formulations.

TABLE 20.2
Kinetic Details for Formulations Prepared with
Varying HLB liquid Pluronic Mobile Phases

| | % | | | Time to |
|---|---|---|---|---|
| Pluronic | Delivered | RR | RR | target rate |
| Used | at 72 hrs. | @ 48 hrs. | @ 72 hrs. | 1.8 mg/hr |
| L-35 | 84% | 1.7 | 0.8 | 46 |
| L-44 | 91% | 1.7 | 0.9 | 46 |
| L-62 | 78% | 1.7 | 1.2 | 45 |
| L-64 | 92% | 2.0 | 0.3 | 51 |
| P-85 | 86% | 1.5 | 0.5 | 45 |
| L-121 | 61% | 1.1 | 0.8 | 35 |
| 25R4 | 73% | 1.5 | 0.9 | 42 |

### Example 21

### Tocopherol Succinate Burst Vs Sustained Release

This example demonstrates how altering the hydrophobicity of the liquid vehicle can alter the drug release kinetics from the matrix. Specifically, two tocopherol esters, tocopherol acetate and tocopherol succinate were used to formulate putties which had all other components held constant as shown in Table 21.1 below.

TABLE 21.1

| Formulation | Calcium | Tocopherol | Tocopherol | Lidocaine |
|---|---|---|---|---|
| # | Stearate | Succinate | Acetate | Free Base |
| 1 | 55% | | 29% | 16% |
| 2 | 55% | 29% | | 16% |

The decreased hydrophobicity of tocopherol succinate over tocopherol acetate creates less of a barrier for the penetration of water based solvents into the matrix. As the aqueous solvent penetrates the matrix there is an increased area for exchange of drug from the matrix into the aqueous milieu. Furthermore, the more hydrophilic tocopherol creates a drug reservoir which is more easily mobilized out of the putty and into the aqueous milieu. As shown in Table 21.2, these properties allow higher drug delivery rates at 24 hours and also leave less total drug behind after 72 hours. By extricating more drug, the lower hydrophobicity reservoir is able to maintain a therapeutic concentration for a longer period of time than the more hydrophobic reservoir containing putty.

TABLE 21.2

| | % | | | Time to |
|---|---|---|---|---|
| | released | R. Rate | R. Rate | LFB RR |
| Putty Formulation | @ 72 hrs. | @ 48 hrs. | @ 72 hrs. | 1.8 mg/hr |
| 55% CS/29% Toc. | 63% | 1.4 | 0.7 | 39 |
| Succinate/16% LFB | | | | |
| 55% CS/29% TA/16% | 40% | 0.8 | 0.5 | 19 |
| LFB | | | | |

In another example of the utility of altering the hydrophobicity of the reservoir, the two tocopherol esters are combined with an alkylene oxide copolymer, Pluronic L-35, to create a matrix with a reservoir (tocopherol) and a mobile phase (Pluronic L-121).

TABLE 21.3

| Sample | Calcium | Pluronic | Tocopherol | Tocopherol | Lidocaine |
|---|---|---|---|---|---|
| # | Stearate | L-121 | Succinate | Acetate | Free Base |
| 1 | 55% | 24% | | 5% | 16% |
| 2 | 55% | 24% | 5% | | 16% |

The addition of the mobile phase to the reservoir containing putty allowed for increased drug mobilization in both cases compared to the putties described in Table 21.4 which did not contain a mobile phase.

TABLE 21.4

| | | | | Time to |
|---|---|---|---|---|
| Sample | % at | R. Rate | R. Rate | LFB RR |
| # | 72 hrs. | @ 48 hrs. | @ 72 hrs. | 1.8 mg/hr |
| 1 | 61% | 1.1 | 0.8 | 32 |
| 2 | 78% | 1.3 | 0.9 | 37 |

### Example 23

### Delivery of Lyophilized Protein

A drug delivery putty is created comprising, by weight, 65% micronized magnesium palmitate as component 1, 35% nonanol as component 2, 5% vitamin K as component 3 and 0.1% GDF-5, which is solubilized in components 2 and 3.

The putty is formulated with a hydrophobic liquid vehicle such as, for example, Pluronic, TEC or Decanol comprising between about 1 and 40% of the putty by mass, a therapeutic protein comprising up to about 10% of the putty by mass and a solid vehicle such as, for example, calcium stearate, calcium laurate or high MW polyethylene glycol comprising up to about 70% of the putty by mass.

The therapeutic protein, e.g., BMP-2, BMP-7 or GDF-5, is lyophilized and added to the liquid vehicle during putty formulation. The liquid vehicle acts as a partition controlling the release of the therapeutic protein thereby prolonging therapeutic benefit. The liquid also acts to stabilize the protein by providing an anhydrous environment. The formulation can then be tested for efficacy in ectopic bone formation as described in example 25.

Angiogenic factors or agents that contribute to angiogenesis include, but are not limited to, VEGF (Vascular Endothelial Growth Factor), angiopoietins (Ang1 and Ang2), members of the matrix metalloproteinase (MMP) family, fibroblast growth factor-2 (FGF2 or bFGF), platelet derived growth factor (PDGF), Delta-like ligand 4 (DII4) and Pleiotrophin. Any combination of angiogenic factors or agents that contribute to angiogenesis are also delivered if desired.

BMPs include, for example, BMP2, BMP3, BMP4, BMPS, BMP6, BMP7, BMP8a, BMP8b, BMP10 and BMP15. Growth differentiation factors (GDFs) include, for example, GDF1, GDF2, GDF3, GDF5, GDF6, GDF7, Myostatin/GDF8, GDF9, GDF10, GDF11 and GDF15.

Pro-proliferative factors include, but are not limited to, epidermal growth factor (EGF), members of the fibroblast growth factory (FGF) family, granulocyte-macrophage colony-stimulating factor (GMCSF), and transforming growth factor (3 (TGF-pl and TGF-(32).

Proteins that increase matrix production include, but are not limited to, insulin-like growth factors I and II. Chemotactive agents or cell migration inductors include, but are not limited to, granulocyte-macrophage colony-stimulating factor (GMCSF), nerve growth factor, transforming growth factor-(3 (TGF-131 and TGF-(32). In another preferred embodiment, a statin is mixed with the carrier of Example 26 at ratios (wt/wt) of 1 part statin to 99 parts carrier, 5 parts statin plus 95 parts carrier, 10 parts statin to 90 parts putty and 20 parts satin to 80 parts carrier.

### Example 24

### Example of the Osteoinductive Potential of Hemostatic Putties Containing BMP2

An osteoinductive agent, such as BMP-2, can be added to a formulation containing a bulk solid, liquid vehicle and drug reservoir. An example of such a formulation with a total weight of 2 grams might contain 1.195 grams of calcium stearate, 0.7 grams of Pluronic L-35, 0.1 grams of tocopherol acetate and 5 milligrams of BMP-2. The resulting formulation would be expected to provide controlled and sustained release of the osteoinductive agent. The osteoinductive potential of such a formulation would be tested in an athymic rat or mouse intramuscular or subcutaneous implantation model. In these models, the osteoinductive agent would induce ectopic bone formation which could be measured radiographically or by histological evaluation at 30 days.

### Example 25

### Further Characterization of Elution Properties

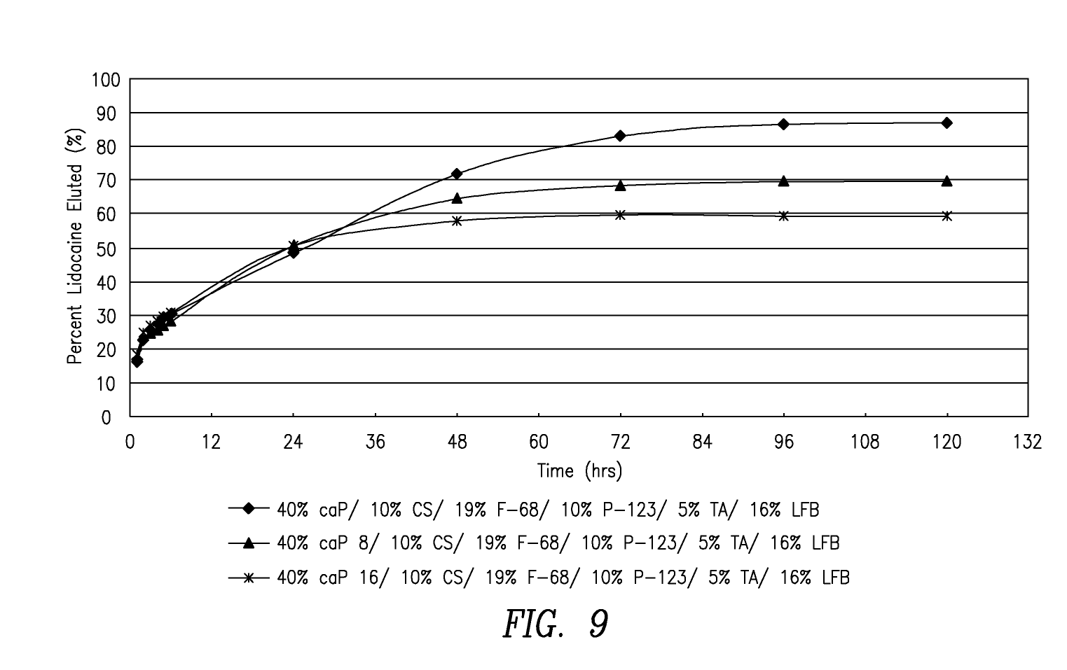

FIG. 9 represents the elution rate selected formulations over time.

TABLE 25.1
HLB and Eutectic Putties

| | | | | |
|---|---|---|---|---|
| | % | | | Time to |
| | released | RR | RR | LFB RR |
| Putty Formulation | at 72 hrs. | @ 48 hrs. | @ 72 hrs. | 1.8 mg/hr |

Control

| | | | | |
|---|---|---|---|---|
| Orthostat-L (Control) | 84% | 1.7 | 0.8 | 46 |
| 55% CS/24% Pluronic | 75% | 1.5 | 0.9 | 42 |
| L-61/5% TA/16% LFB | | | | |
| 55% CS/24% Pluronic | 70% | 1.3 | 0.8 | 42 |
| L-92/5% TA/16% LFB | | | | |
| 55% CS/24% Pluronic - | 73% | 1.5 | 1 | 40 |
| 101/5% TA/16% LFB | | | | |
| 55% CS/24% Pluronic | 70% | 1.3 | 0.8 | 42 |
| L-103/5% TA/16% LFB | | | | |
| 38% CS/43% Toc. Succ/ | 65% | 1.7 | 0.7 | 47 |
| 16% LFB | | | | |
| 55% CS/24% Pluronic | 79% | 1.6 | 0.9 | 38 |
| L-81/5% TA/16% LFB | | | | |

TABLE 25.2
Tocopherol Derivates with Pluronics,
Solid Pluronics, and Lidocaine HCl

| | | | | |
|---|---|---|---|---|
| | % | | | Time to |
| | released | RR | RR | LFB RR |
| Putty Formulation | at 72 hrs | @ 48 hrs | @ 72 hrs | 1.8 mg/hr |

Control

| | | | | |
|---|---|---|---|---|
| Orthostat-L (Control) | 84% | 1.7 | 0.8 | 46 |

Tocopherol Derivatives and Pluronics

| | | | | |
|---|---|---|---|---|
| 55% CS/9% L-121/10% | 91% | 1.9 | 1.0 | 51 |
| TA/10% TPGS/16% LFB | | | | |
| 55% CS/24% L-121/5% | 65% | 1.3 | 0.9 | 37 |
| TS/16% LFB | | | | |
| 55% CS/14% L-121/15% | 59% | 1.1 | 1.1 | 30 |
| TS/16% LFB | | | | |
| 55% CS/19% L-44/10% | 89% | 1.9 | 0.8 | 50 |
| TS/16% LFB | | | | |
| 55% CS/9% L-44/10% | 91% | 1.5 | 0.7 | 43 |
| TA/10% TPGS/16% LFB | | | | |
| 55% CS/19% L-35/5% | 93% | 1.0 | 0.1 | 42 |
| TA/5% TS/16% LFB | | | | |

Lidocaine HCl Formulations

| | | | | |
|---|---|---|---|---|
| 55% CS/29% L-35/16% | 87% | 1.7 | 0.9 | 46 |
| LFB | | | | |
| 55% CS/29% TA/16% | 13% | 0.2 | 0.1 | 3 |
| HCl | | | | |
| 55% CS/24% L-35/5% | 81% | 0.8 | 0.5 | 35 |
| TA/8% LFB/8% HCl | | | | |
| 55% CS/24% TEC/5% | 83% | 1.5 | 0.3 | 45 |
| TA/8% LFB/8% HCl | | | | |
| 47% CS/37% TEC/16% | 74% | 0.1 | 0.0 | 19 |
| HCl (holder) | | | | |
| 47% CS/37% TEC/16% | 74% | 0.0 | 0.0 | 21 |
| HCl (disc) | | | | |

Solid Pluronics

| | | | | |
|---|---|---|---|---|
| 55% CL/12.5% P-123/ | 91% | 2.1 | 0.9 | 54 |
| 9% F-68/7.5% TA/16% | | | | |
| FB | | | | |
| 50% CS/14.5% P-123/ | 76% | 1.4 | 0.6 | 42 |
| 14.5% F-68/5% TA/16% | | | | |
| LFB | | | | |

TABLE 25.3
HLB and Tocopherol Putties

| | | | | |
|---|---|---|---|---|
| | % | | | Time to |
| | released | RR | RR | LFB RR |
| Putty Formulation | at 72 hrs. | @ 48 hrs. | @ 72 hrs. | 1.8 mg/hr |

Control

| | | | | |
|---|---|---|---|---|
| Orthostat-L (Control) | 84% | 1.7 | 0.8 | 46 |
| 55% CS/P1 L-121/10% | 63% | 1.2 | 0.8 | 34 |
| TA/16% LFB | | | | |
| 55% CS/19% P1 L-62/ | 79% | 1.5 | 0.9 | 43 |
| 10% TA/16% LFB | | | | |
| 55% CS/19% P1 L-64/ | 90% | 1.7 | 1 | 46 |
| 10% TA/16% LFB | | | | |
| 55% CS/9% P1 L-35/ | 83% | 1.3 | 0.7 | 42 |
| 10% TPGS/10% TA/16% | | | | |
| LFB | | | | |
| 55% CS/19% TPGS/10% | 95% | 1.5 | 0.3 | 45 |
| TA/16% LFB | | | | |
| 55% CS/24% P1 L-35/ | 93% | 1.4 | 0.7 | 45 |
| 5% TPGS/16% LFB | | | | |

TABLE 25.4
Mobile Phases with Single/Double Tocopherol Derivatives

| | | | |
|---|---|---|---|
| | % | | Time to |
| | released | RR | LFB RR |
| Putty Formulation | at 72 hrs. | @ 72 hrs. | 1.8 mg/hr |

Control

| | | | |
|---|---|---|---|
| Orthostat-L (Control) | 84% | 0.8 | 46 |

Single Tocopherol Derivative

| | | | |
|---|---|---|---|
| 55% CS/14% L-35/7.5% TS/ | 78% | 0.9 | 46 |
| 7.5% TA/16% LFB | | | |
| 55% CS/19% L-121/10% TS/ | 67% | 1.2 | 35 |
| 16% LFB | | | |
| 55% CS/19% L-64/10% TS/ | 83% | 1.0 | 53 |
| 16% LFB | | | |

Double Tocopherol Derivative

| | | | |
|---|---|---|---|
| 55% CS/24% L-35/5% TA/16% | 89% | 0.9 | 47 |
| LFB | | | |
| 45% CS/10% PEG 2000/14% L- | 72% | 1.4 | 40 |
| 44/15% TA/16% LFB | | | |
| 55% CS/9% L-64/10% TPGS/ | 93% | 0.6 | 47 |
| 10% TA/16% LFB | | | |

Liquid Mobile Phase

| | | | |
|---|---|---|---|
| 55% CS/24% P L-62/5% TOC/ | 78% | 1.2 | 46 |
| 16% LFB | | | |
| 55% CS/24% P L-64/5% TOC/ | 92% | 0.3 | 52 |
| 16% LFB | | | |
| 55% CS/24% P P-85/5% TOC/ | 86% | 0.5 | 45 |
| 16% LFB | | | |
| 55% CS/24% P L-121/5% TOC/ | 61% | 0.8 | 32 |
| 16% LFB | | | |
| 55% CS/24% P 25R4/5% TOC/ | 73% | 0.9 | 42 |
| 16% LFB | | | |

TABLE 25.5
Formulations containing TCP, Solid
Pluronics, PEG and/or Dispersants

| | | | | Time to |
|---|---|---|---|---|
| | % at | R. Rate | R. Rate | LFB RR |
| Putty Formulation | 72 hrs. | @ 48 hrs. | @ 72 hrs. | 1.0 mg/hr |
| Orthostat-L (Control) | 84% | 1.7 | 0.8 | 68 |
| 50% TCP/16% P-123/8% | 84% | 2.5 | 0.9 | 70 |
| F-127/10% TA/16% LFB | | | | |
| 10% CS/40% TCP/10% P- | 77% | 2.8 | 2.0 | 2.0 @ 70 |
| 123/14% F-68/10% TA/ | | | | |
| 16% LFB | | | | |
| 50% CS/19% P-123/10% | 83% | 1.7 | 0.9 | 65 |
| F-68/5% TA/16% LFB | | | | |
| 35% CS/31% C. Succ/ | 48% | 1.3 | 0.8 | 62 |
| 13% L-35/5% TA/16% | | | | |
| LFB | | | | |
| 40% CS/15% CL-M/29% | 66% | 1.4 | 1.1 | 1.1 @ 72 |
| TA/16% | | | | |
| 10% CS/45% TCP/16% | 63% | 2.1 | 1.8 | 1.8 @ 72 |
| PEG 1000/8% PEG | | | | |
| 2000/5% TA/16% LFB | | | | |
| 55% CS/24% P-85/5% | 94% | 1.6 | 0.3 | 57 |
| TA/16% LFB | | | | |
| 55% CS/24% P-103/5% | 83% | 1.8 | 1.1 | 1.1 @ 72 |
| TA/16% LFB | | | | |
| 50% CS/12% PEG 900/ | 68% | 1.4 | 1.0 | 72 |
| 12% PEG 2000/10% TA/ | | | | |
| 16% LFB | | | | |
| 50% TCP/12% PEG 900/ | 7% | 0.2 | 0.0 | 10 |
| 12% PEG 2000/10% TA/ | | | | |
| 16% LFB | | | | |
| 29% CS/29% GPCS/16% | 63% | 1.2 | 0.8 | 59 |
| L-35/10% TA/16% LFB | | | | |
| 25% CS/34% GPCS/25% | 54% | 1.3 | 1.0 | 72 |
| TA/16% LFB | | | | |

TABLE 25.6
A summary of ormulations that maximize release rate at 72 hours

| | % LFB | Rate | Time to |
|---|---|---|---|
| | Eluted | @ 72 hrs | Target - 1.8 |
| | @ 72 hrs | (mg/hr) | mg/hr (Hrs) |
| 10% CS/40% TCP/10% P-123/ | 77% | 2.0 | >72 |
| 14% F-68/10% TA/16% LFB | | | |
| 40% CS/15% CL-M/29% | 66% | 1.1 | 38 |
| TA/16% LFB | | | |
| 55% CS/24% P-103/5% | 83% | 1.1 | 48 |
| TA/16% LFB | | | |
| 50% CS/12% PEG 900/12% | 68% | 1.0 | 36 |
| PEG 2000/10% TA/16% LFB | | | |

TABLE 25.7
Formulations that maximize duration of efficacy

| | % LFB | Rate | Time to |
|---|---|---|---|
| | Eluted | @ 72 hrs | Target - 1.8 |
| | @ 72 hrs | (mg/hr) | mg/hr (Hrs) |
| Orthostat-L | 81% | 0.8 | 44 |
| 10% CS/40% TCP/10% P-123/ | 77% | 2.0 | >72 |
| 14% F-68/10% TA/16% LFB | | | |
| 10% CS/45% TCP/16% PEG | 63% | 1.8 | 69 |
| 1000/8% PEG 2000/5% TA/ | | | |
| 16% LFB | | | |
| 25% CA/30% CS/29% TA/ | 92% | 0.5 | 59 |
| 16% LFB | | | |
| 25% CA/30% MS/24% L-35/ | 93% | 0.3 | 54 |
| 5% TA/16% LFB | | | |

### Example 26

### Osteoconductive Formulations as Long Term Drug Reservoirs

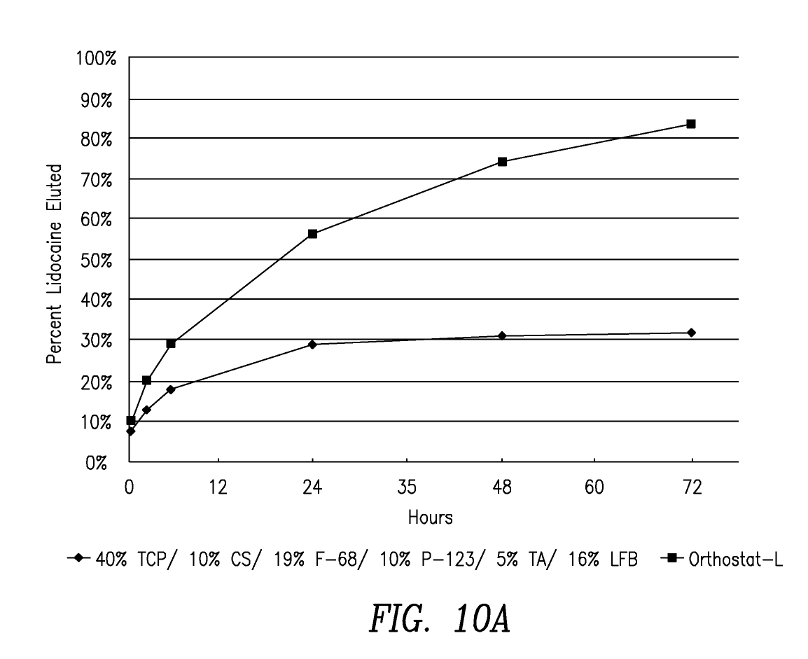

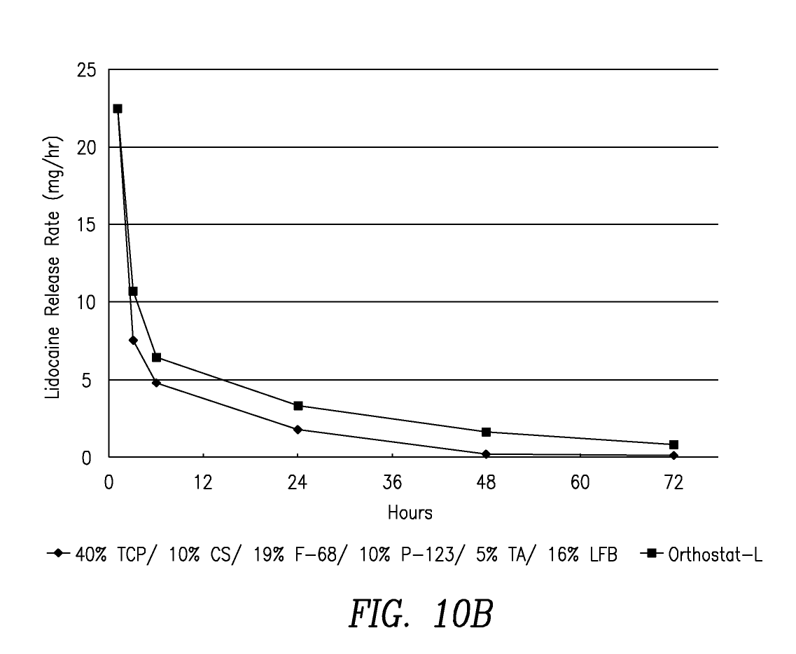

In this example, a formulation capable of controlled release of hydrophobic drugs including lidocaine was prepared using an osteoconductive material, tricalcium phosphate (TCP) as part of the solid bulk matrix. Formulations containing osteoconductive material(s) and a blend of polymers including alkylene oxide copolymers and PEGs of varying molecular weight can be used to create osteoconductive hemostats which also can serve as long term drug reservoirs. A formulation comprised of 40% TCP, 10% calcium stearate, 19% Pluronic F-68, 10% Pluronic P-123, 5% tocopherol acetate and 16% lidocaine was prepared and tested for lidocaine elution. This formulation demonstrated slow drug elution, with 67% of the lidocaine remaining at 72 hours as compared to less than 20% remaining in the Orthostat-L formulation. Since TCP is remodeled into host bone as part of a long term process known to take several months, long term individual or concomitant drug release over the duration of the remodeling process could be useful in improving patient outcomes. This data is represented in FIGS. 10A and 10B.

### Example 27

### Preparation of Ionic Polymers and their Incorporation into the Inventive Putties

See Example 11 for details of elution testing in an elution bath. “Orthostat-L” refers to the base carrier plus 16% lidocaine.

Structurally, alginic acid is a linear copolymer with homopolymeric blocks of (1-4)-linked beta-D-mannuronate (M) and its C-5 epimer, alpha-L-guluronate (G) residues, respectively, covalently linked together in different sequences or blocks. The monomers can appear in homopolymeric blocks of consecutive G residues (G-blocks), consecutive M residues (M-blocks), alternating M- and G-residues (MG-blocks) or randomly organized blocks.

It was determined experimentally, as well as from the literature, that both alginic acid, NF (extracted from the cell walls of brown seaweed), and synthetic lidocaine free base, USP, each are virtually insoluble in distilled water. Stirred overnight at room temperature, a suspension of equimolar quantities (with respect to the calculated number of free amino and carboxyl groups contained in each compound), of lidocaine free base and alginic acid in distilled water resulted in a soluble, viscous, hazy, brownish solution of lidocaine alginate which could be precipitated from solution as a clear hydrogel by the drop-wise addition of an aqueous solution of calcium chloride to a stirred solution of lidocaine alginate. The precipitated hydrogel was not chemically analyzed but application to a lower lip caused the rapid onset of localized numbness.

### Experimental:

A series of lidocaine alginate solutions, varying in mole-% lidocaine with respect to alginic acid, was prepared according to the following table. The stochiometry was based on the assumption that there is one free carboxyl group per alginic acid glycoside residue, [(C₆H₈O₆)ₙ, MW_{monomer unit}=176, polymer acid value=230 (min.)] and one free tertiary amino group per lidocaine free base molecule (MW=234).

TABLE 27.1

| | Mole-% Lidocaine in Lidocaine Alginate (In 25 ml. Water) | | | | | | |
|---|---|---|---|---|---|---|---|
| | 10 | 25 | 40 | 55 | 70 | 85 | 100 |
| Alginic acid, g. | 1.76 | 1.76 | 1.76 | 1.76 | 1.76 | 1.76 | 1.76 |
| Lidocain g. | 0.234 | 0.585 | 0.936 | 1.287 | 1.638 | 1.989 | 2.34 |
| mole % carboxyl | 90 | 75 | 60 | 45 | 30 | 15 | 0 |
| free carboxyl | 75 | 60 | 45 | 30 | 15 | 0 | 0 |
| mole % Ca⁺²/2 | 0 | 15 | 15 | 15 | 15 | 0 | 0 |

It was decided to conduct further experiments using batches of 85 mole-% lidocaine alginate solutions (ratio based on 1.76 g. alginic acid+1.99 g. lidocaine free base in 25 ml. water). Two batches of 85 mole-% lidocaine alginate were prepared by stirring, overnight, 9.9 g. of lidocaine free base with 8.8 g. of alginic acid, NF in 125 ml. of water.

For initial crosslinking experiments, calcium chloride dihydrate (MW=146) was selected as the crosslinking agent. An aqueous stock solution of calcium chloride dihydrate was prepared containing 22 mg. CaCl₂.2H₂O in 5 ml. of water. With stirring, 10 ml. of the calcium chloride stock solution was added drop-wise to 10 ml. of 85 mole-% lidocaine alginate solution. An almost clear hydrogel was rapidly deposited from which excess fluid was expressed. The hydrogel was washed once with 10 ml. of distilled water and dried overnight on a watch glass in air. The resulting horn-like product was ground into a fine powder (Solid Lidocaine Alginate Powder, SLAP) at room temperature in a commercial electric coffee mill. Particle size was not measured but the material was not micronized.

Two putties were prepared in which 15% of the calcium stearate was replaced with the same weight of SLAP. Putty 1 represents a Orthostat-L formulation while Putty 2 represents Orthostat. Detailed putty compositions are given below:

| | |
|---|---|
| Putty 1 | Putty 2 |

| | | | | |
|---|---|---|---|---|
| Calcium Stearate | 40.0% | 2.00 g. | 45.0% | 2.25 g. |
| Plutonic L-35 | 23.4 | 1.17 | 35.0 | 1.75 |
| Tocopheryl Acetate | 5.2 | 0.26 | 5.0 | 0.25 |
| Lidocaine Free Base | 16.4 | 0.82 | — | — |
| SLAP | 15.0 | 0.75 | 15.0 | 0.75 |

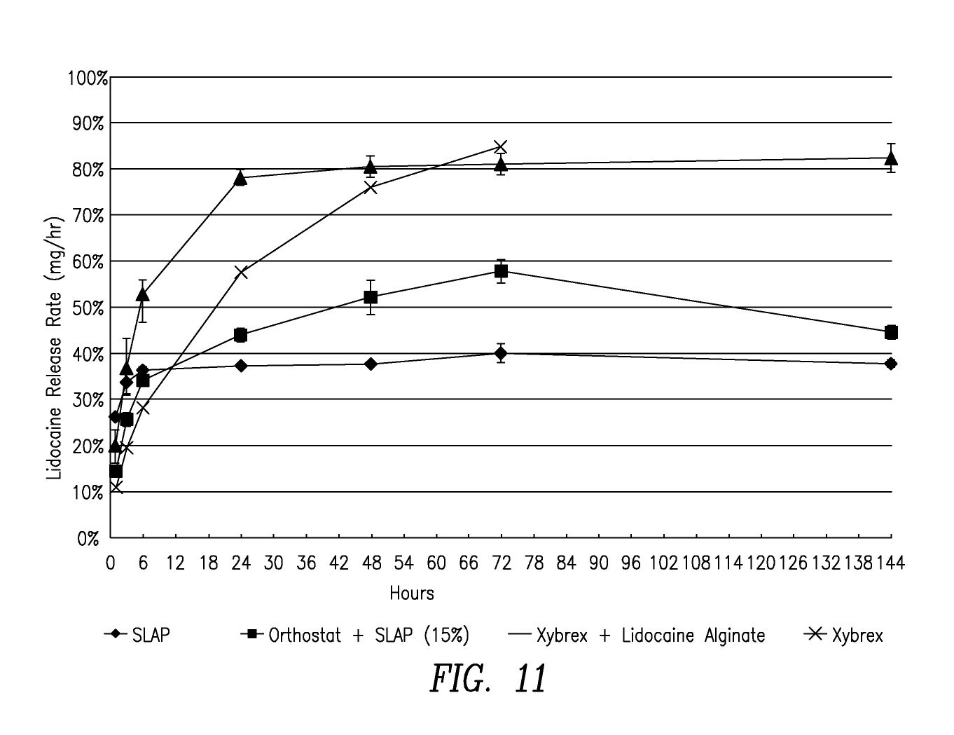

Both Putty 1 and Putty 2 were considerably less firm in consistency than Orthostat-L and Orthostat. This is expected because a portion of the bulking micronized calcium stearate was replaced with non-micronized SLAP. Duplicate samples of Putty 1 and Putty 2 as well as duplicate samples of 1.0 g. of SLAP, each contained in individual biopsy bags, were placed in slowly stirred, pH 7.4 phosphate buffer (900 ml.) Diso bath actinic containers at 37° C. Five ml. samples were taken for HPLC lidocaine analysis from each container at 1, 2, 6, 24, 48, 72 and 144 hours. The samples were immediately replaced with 5 ml. of fresh phosphate buffer to maintain a constant bath volume.
The Diso bath samples, mentioned above, were filtered and analyzed for released lidocaine using the standard Orthocon HPLC method. The results are tabulated below together with values for a typical Orthostat-L sample in Tables 27.2 and 27.3. These values are graphically represented in FIG. 11.

TABLE 27.2
Percent Lidocaine Eluted

| | | 1 | 3 | 6 | 24 | 48 | 72 | 144 |
|---|---|---|---|---|---|---|---|---|
| Lidocaine | Avg | 26% | 34% | 36% | 37% | 38% | 40% | 38% |
| Alginate | sd | 0.01 | 0.02 | 0.01 | 0.00 | 0.00 | 0.02 | 0.01 |
| (neat) | | | | | | | | |
| Orthostat + | Avg | 14.4% | 25.6% | 34.0% | 43.9% | 52.1% | 57.7% | 44.5% |
| Lidocaine | sd | 0.00 | 0.01 | 0.00 | 0.02 | 0.04 | 0.03 | 0.02 |
| Alginate | | | | | | | | |
| Orthostat-L + | Avg | 19.8% | 37.1% | 53.0% | 78.1% | 80.5% | 81.0% | 82.3% |
| Lidocaine | sd | 0.04 | 0.06 | 0.06 | 0.02 | 0.02 | 0.02 | 0.03 |
| Alginate | | | | | | | | |
| Orthostat-L | — | 10.8% | 19.5% | 28.1% | 57.6% | 76.0% | 84.9% | NA |
| (n = 1) | | | | | | | | |

TABLE 27.3
Lidocaine Release Rate (mg/hr)

| | | 1 | 3 | 6 | 24 | 48 | 72 | 144 |
|---|---|---|---|---|---|---|---|---|
| Lidocaine | Avg | 144.0 | 20.7 | 4.7 | 0.3 | 0.1 | 0.6 | −0.5 |
| Alginate | sd | 6.0 | 2.7 | 3.0 | 0.2 | 0.0 | 0.6 | 0.3 |
| (neat) | | | | | | | | |
| Orthostat + | Avg | 15.3 | 5.9 | 3.0 | 0.6 | 0.4 | 0.2 | −0.6 |
| Lidocaine | sd | 0.0 | 0.6 | 0.5 | 0.1 | 0.1 | 0.3 | 0.2 |
| Alginate | | | | | | | | |
| Orthostat-L + | Avg | 52.2 | 22.9 | 14.2 | 3.8 | 0.3 | 0.1 | 0.1 |
| Lidocaine | sd | 3.2 | 0.7 | 1.6 | 1.1 | 0.0 | 0.0 | 0.1 |
| Alginate | | | | | | | | |
| Orthostat-L | — | 22.6 | 9.1 | 6.0 | 3.4 | 1.6 | 0.8 | NA |
| (n = 1) | | | | | | | | |

Discussion: While the composition of SLAP (diamonds, above) was not measured directly as part of this experiment, about 50% of its weight was assumed to be lidocaine. Therefore, 1.0 g. samples of SLAP, each theoretically containing about 500 mg. of lidocaine, was extracted in the Diso bath. After 6 hours, 56% of the original 1 g. sample weight dissolved, analyzed as lidocaine and represented the release of 278 mg. of lidocaine rather than the assumed theoretical quantity of 500 mg. Thus, it may be concluded that 278 mg. rather than 500 mg. of lidocaine (56% of the assumed theoretical amount) was releasable from 1,000 mg. of SLAP material during the first 6 hours of extraction. This quantity of lidocaine, released from SLAP, is considered experimentally determined.

Summary—SLAP: A. Diso bath extraction of SLAP in phosphate buffer dissolved 56% of the theoretical quantity of lidocaine alginate.

### B. This Experimentally Determined Composition of SLAP was Used for all Subsequent Calculations.

For the Orthostat+SLAP samples (squares), 15% of the calcium stearate was replaced with 15% of SLAP, as described above. An average of 1.35 g. of Putty 2, containing 203 mg. (15%) of SLAP and the experimentally determined total of 113.7 mg. (56%×203 mg.) of lidocaine was extracted in the Diso bath using standard phosphate buffer. After 6 hours, 35% of the original SLAP material was solubilized, equivalent to a total of 39.8 mg. of lidocaine. During the next 56 hours (72 hour time-point) an additional 24% of lidocaine was extracted, representing another 27.4 mg. of lidocaine for a total of 67.2 mg or a total of 59% (vs. 56% extracted during the first 6 hours from pure SLAP) of the original lidocaine was extracted over 72 hours.

Summary−Orthostat+SLAP: A. As expected, suspending particulate SLAP in Orthostat putty significantly slows the rate at which lidocaine is released.

B. The Total Amount of Lidocaine Released During 72 Hours (59%) Reasonably Agrees with the Total Amount Released from Pure SLAP (56%) after Six Hours.
For the Orthostat-L+SLAP samples (triangles), 15% of the calcium stearate was replaced with 15% of SLAP, as described above. An average of 1.1 g. of Putty 1, containing 165 mg. (15%) of SLAP and an experimentally determined (6 hour time-point) total (from SLAP) of 92.4 mg. (56%×165 mg.) of lidocaine plus 165 mg. (15%×1.1 g) of lidocaine free base for a total of 257.4 mg (92.4 mg. elutable at six hours from SLAP and 165 mg. from Orthostat-L) of lidocaine. After six hours in the Diso bath buffer, 53% of the total amount of lidocaine or 136.4 mg. of lidocaine was extracted. If it is assumed that the SLAP eluted 100% of its six hour time-point elutable lidocaine content of 92.4 mg of lidocaine and since a total of 136.4 mg. was extracted, 43.6 mg. (136.4-92.4) of lidocaine must be attributed to release of free base from the putty. Examining the average release rate curve for Orthostat-L (X's), it is found that 28% of the original amount of lidocaine free base added to Orthostat-L is released at the six hour time-point, equivalent, in this case, to 46.2 (165×28%) mg of lidocaine. extracted from normal Orthostat-L. Thus, 46.2 mg. of lidocaine (in reasonable agreement with previous Orthostat-L release data=49.3 mg). from the free base contained in the Orthostat-L putty plus 92.4 mg of lidocaine eluted from SLAP after six hours or a total of 138.6 mg of lidocaine eluted from the Orthostat-L-SLAP mixture after six hours. After 24 hours of Diso bath extraction, an additional 25.1% (64.3 mg.) of lidocaine was released for a total of 200.7 mg, (136.4+64.3) or 78% (200.7/257.4) of the original Putty 1 lidocaine content.
Summary−Orthostat-L+SLAP: A. As expected, over the initial 24 hours of extraction, significantly more lidocaine is released as a result of the addition of SLAP to Orthostat-LB.

### REFERENCES

Batrakova, et al., (2003) Optimal Structure Requirements for Pluronic Block Copolymers in Modifying P-glycoprotein Drug Efflux Transporter Activity in Bovine Brain Microvessel Endothelial Cells. J. PHARMACOLOGY AND EXPERIMENTAL THERAPEUTICS Vol. 304, No. 2845-854.
Gandhi, A., C Wang, A Djalali, D Knaack, J Katz, G Strichartz, P Gerner (2008) An Absorbable Local Anesthetic MatrixProvides a Dose-Dependent SciaticNerve Blockade: Correlation of In Vitro Lidocaine Release Kinetics to In Vivo. Abstract #137 35th Annual Meeting & Exposition of the Controlled Release Society.
Giron, D. (2003) Characteristics of Salts of Drug Substances. J. of Thermal Analysis and calorimetry 73; 441-447.
Knaack et al. U.S. Pat. No. 6,500,516.
Kronenthal: PCT/200610288231; PCT/US2004/026739; PCT/US2004/026738.
Lee et al. U.S. Pat. No. 6,541,037.
Serajuddin, Abu T. M. (2007) Salt formation to improve drug solubility. Adv Drug Delivery Rev. 59: 603-616.
Vaughn, Mark Nace Nonionic Surfactants: Polyoxyalkylene Block Copolymers (1996) CRC Press.
Wang C F, Djalali A G, Gandhi A, Knaack D, De Girolami U, Strichartz G, Gerner P. An absorbable local anesthetic matrix provides several days of functional sciatic nerve blockade. Anesth Analg. 2009 March; 108(3):1027-33.
Winterbottom et al. WO20050251267.

### Equivalents

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A drug delivery carrier comprising a suspension of a micronized solid and a mobile phase that is a solid or liquid having low water solubility, wherein the micronized solid is substantially insoluble in the mobile phase and makes up between 10 and 70% of the composition, and wherein the mobile phase comprises a surfactant having a hydrophile-lipophile balance (HLB) value that is less than 17,
wherein the elution rate of a drug substance from the carrier in an in vitro test is greater than 1.0 mg/hr for at least 45 hours under conditions in which the carrier is mixed with 16% (wt/wt) of the drug substance and eluted for 72 hours in a phosphate buffer at 37 C.

2. The drug delivery carrier of claim 1, wherein the drug substance in the in vitro test is lidocaine free base.

3. The drug delivery carrier of claim 1 or 2, wherein the surfactant is a neutral surfactant.

4. The drug delivery carrier of claim 3, wherein the neutral surfactant is one or more surfactants selected from the group consisting of sorbitan, polyacrylates, alkoxylated fatty alcohols, block copolymers of polypropylene oxide and ethylene oxide, ethylene glycol, propylene glycol, blocked polymers (e.g., Chemal BP 261), silicon glycol co-polymers, polyoxyethylene ethers, ethoxylated triglycerides, ethoxylated fatty acids, ethoxylated fatty amines, and derivatives and modifications of any of the foregoing.

5. The drug delivery carrier of claim 4, wherein the neutral surfactant is an alkylene oxide block copolymer selected from the group consisting of Pluronic L-10, Pluronic L-43, Pluronic L-44, Pluronic L-61, Pluronic L-62, Pluronic 17R2, and Pluronic L-92.

6. The drug delivery carrier of claim 3, wherein the neutral surfactant is a liquid with a critical micelle concentration (CMC) of less than 1%.

7. The drug delivery carrier of claim 6, wherein the surfactant is selected from the group consisting of poloxamers, sorbitan derivatives and ethoxylated fatty acids.

8. The drug delivery carrier of any one of claims 1-7, wherein the mobile phase comprises a poloxamer.

9. The drug delivery carrier of any one of claims 1-8, wherein the mobile phase comprises a reservoir having low water solubility.

10. The drug delivery carrier of claim 9, wherein the reservoir is selected from the group consisting of sucrose acetate isobutyrate (SAIB), alpha-tocopherol acetate, α-tocopherol, pegylated tocopherol succinate, sorbitan oleate, sorbitan laurate, vitamin K1, cholesterol, fatty acid esters, block copolymers of polypropylene oxide and ethylene oxide (e.g., Pluronic L-31, Pluronic L-81, Pluronic L-101, Pluronic 31R1, Pluronic L-121), alkoxylated fatty alcohols, sorbitan trioleate, and polypropylene glycol 2000.

11. The drug delivery carrier of any one of claims 1-10, wherein the micronized solid is selected from the group consisting of an anionic material, a cationic material, or a porous material.

12. The drug delivery carrier of claim 11, wherein the micronized solid is an anionic material and wherein the anionic moiety is selected from the group consisting of carboxylates, phosphates, sulfates, carbonates, phosphonates, silicates, and chlorates.

13. The drug delivery carrier of claim 11, wherein the micronized solid is an cationic material and wherein the cationic moiety is selected from the group consisting of amines, ammonium, and choline.

14. The drug delivery carrier of claim 11, wherein the micronized solid is a porous material selected from the group consisting of ceramics, polysaccharides, fatty acid salts and polyamines.

15. The drug delivery carrier of claim 14, wherein the porous material is a calcium, magnesium or zinc salt of a fatty acid selected from the group consisting of stearate, palmitate, or laurate.

16. The drug delivery carrier of claim 15, further comprising an embedded solid ceramic material.

17. The drug delivery carrier of any one of claims 1-16, wherein the micronized solid comprises a drug substance in an amount up to 25% by weight of the total weight of the micronized solid.

18. The drug delivery carrier of claim 17, wherein the drug substance is selected from the group consisting of the group consisting of bone growth enhancers, anti-inflammatory agents, anticancer therapeutics, anesthetics, analgesics, antimicrobials, antiseptics, nucleic acids, transcription activators, peptide growth factors, neuropeptides, neuromodulators, hormones, vitamins, and antiarythmics.

19. The drug delivery carrier of claim 18, wherein the drug substance is not an analgesic or an anesthetic.

20. The drug delivery carrier of any one of claims 1-19, further comprising an osteoconductive component.

21. The drug delivery carrier of any one of claims 1-20, further comprising a substantially anhydrous hydrogel forming material.

22. The drug delivery carrier of any one of claims 1-21, further comprising a second drug-containing solid material.

23. The drug delivery carrier of claim 22, wherein the second drug-containing solid material is selected from the group consisting of a porous ceramic, an erodible polymer, a solid surfactant, a substantially anhydrous hydrogel forming material, and a waxy solid.

24. The drug delivery carrier of any one of claims 1-23, wherein less than 84% of the drug substance is eluted within the 72 hours of the in vitro test.

25. The drug delivery carrier of any one of claims 1-23, wherein the burst elution rate of the drug substance during the first hour of release is less than 20 mg/hr in the in vitro test.

26. The drug delivery carrier of any one of claims 1-23, wherein less than 84% of the drug substance is eluted within the 72 hours of the in vitro test and the release rate in the first three days is greater than 40 micrograms per day.

27. A method for delivering a drug substance to a tissue, the method comprising contacting the drug delivery carrier of any one of claims 1-26 containing the drug substance with the tissue, thereby delivering the drug substance to the tissue.

28. The method of claim 27, wherein the tissue is a soft tissue or a hard tissue.

29. A method for reducing or stopping the flow of blood from a tissue, the method comprising contacting the tissue with the drug delivery carrier of any one of claims 1-25, thereby reducing or stopping the flow of blood from the tissue.

30. The method of claim 29, wherein the tissue is a soft tissue or a hard tissue.

31. The method of claim 30, wherein the tissue is a hard tissue and the hard tissue is bone.

32. A method for producing a local nerve block, the method comprising the step of placing the drug delivery carrier of any one of claims 1-26 adjacent to a nerve of interest thereby producing a local nerve block.
